(19) United States
(12) Patent Application Publication
(10) Pub. No.: US 2014/0330018 A1
(43) Pub. Date: Nov. 6, 2014

(54) METATHESIS CATALYSTS AND METHODS THEREOF

(71) Applicants: Massachusetts Institute of Technology, Cambridge, MA (US); Ximo AG, Horw/Lucerne (CH)

(72) Inventors: Janos Czirok, Horw/Lucerne (CH); Georg Frater, Horw/Lucerne (CH); Levente Ondi, Horw/Lucerne (CH); Richard Royce Schrock, Winchester, MA (US); Florian Toth, Horw/Lucerne (CH)

(73) Assignees: Massachusetts Institute of Technology, Cambridge, MA (US); Ximo AG, Horw/Lucerne (CH)

(21) Appl. No.: 14/267,289

(22) Filed: May 1, 2014

Related U.S. Application Data
(60) Provisional application No. 61/818,333, filed on May 1, 2013.

(51) Int. Cl. B01J 31/22; C07C 2/66; C07C 67/30

(52) U.S. Cl. CPC B01J 31/2295; C07C 67/30; C07C 2/66; C07C 2531/12
USPC 546/4; 556/58; 560/122; 585/429

(57) ABSTRACT

The present application provides, among other things, novel compounds and methods for metathesis reactions. In some embodiments, a provided compound has the structure of formula I. In some embodiments, the present invention provides methods for preparing a compound of formula I. In some embodiments, the present invention provides metathesis methods comprising providing a compound of formula I.

### CROSS-REFERENCE TO RELATED APPLICATIONS

The present invention claims priority to U.S. Provisional Application Ser. No. 61/818,333, filed May 1, 2013, the entirety of which is incorporated herein by reference.

### STATEMENT REGARDING FEDERALLY SPONSORED RESEARCH OR DEVELOPMENT

This invention was made with government support under Grant No. CHE1111133 awarded by the National Science Foundation. The government has certain rights in the invention.

### FIELD OF THE INVENTION

The present invention generally relates to metathesis reactions.

### BACKGROUND

Catalytic metathesis has transformed chemical synthesis and offers exceptionally efficient pathways for the synthesis of many commercially important chemicals, including but not limited to biologically active molecules, oleochemicals, renewables, fine chemicals, and polymeric materials. There remains an unmet need for improved methods and catalysts for metathesis reactions, for example, in terms of better catalyst stability and/or activity, efficiency and stereoselectivity.

### SUMMARY

The present invention, among other things, provides new compounds for promoting metathesis reactions. In some embodiments, a provided compound is a stereogenic-at-metal (SAM) complex. In some embodiments, a provide compound has the structure of formula I:

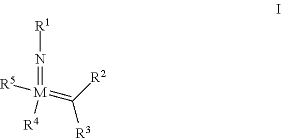

wherein:
M is molybdenum or tungsten;
R¹ is an optionally substituted group selected from C₁₋₂₀ aliphatic, C₁₋₂₀ heteroaliphatic having 1-3 heteroatoms independently selected from nitrogen, oxygen, or sulfur, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
each of R² and R³ is independently R, —OR, —SR, —N(R)₂, —OC(O)R, —SOR, —SO₂R, —SO₂N(R)₂, —C(O)N(R)₂, —NRC(O)R, or —NRSO₂R;
R⁴ is —OR^{s};
R^{s} is —C(R^{t})₂—R′, —Ar^{a}, or an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
each R^{t} is independently halogen or R;
R⁵ is different from R⁴, and is —OR′, —OC(O)R′, —N(R′)₂, or R″;
R′ is hydrogen, —Ar^{a}, or an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R″ is —Ar^{a}, or an optionally substituted group selected from phenyl, an 8-10 membered bicyclic aryl ring, a 5-6 membered monocyclic heteroaryl ring having 1-4 heteroatoms independently selected from nitrogen, oxygen, or sulfur, or an 8-10 membered bicyclic heteroaryl ring having 1-5 heteroatoms independently selected from nitrogen, oxygen, or sulfur;
Ar^{a} is of the following formula:

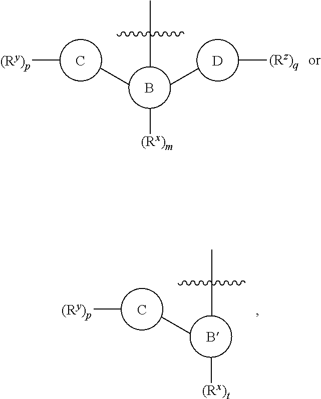

wherein:
m is 0-3;
Ring B is an optionally substituted group selected from phenyl or a 5-6 membered monocyclic heteroaryl ring having 1-4 heteroatoms independently selected from nitrogen, oxygen, or sulfur;
each of p and q is independently 0-5;
t is 0-4;
each of Ring B′, Ring C and Ring D is independently an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-14 membered bicyclic or tricyclic heteroaryl ring having 1-5 heteroatoms independently selected from nitrogen, oxygen, or sulfur;
each of R^{x}, R^{y}, and R^{z} is independently halogen, R, —OR, —SR, —S(O)R, —S(O)₂R, —OSi(R)₃, —N(R)₂, —NRC(O)R, —NRC(O)OR, —NRC(O)N(R)₂, —NRSO₂R, —NRSO₂N(R)₂, or —NROR;
each R is independently hydrogen or an optionally substituted group selected from C₁₋₂₀ aliphatic, C₁₋₂₀ heteroaliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur; or:
   two R groups on the same atom are optionally taken together with the atom to which they are attached to form an optionally substituted 3-10 membered, monocyclic or bicyclic, saturated, partially unsaturated, or aryl ring having, in addition to the atom to which they are attached, 0-4 heteroatoms independently selected from nitrogen, oxygen, or sulfur.

In some embodiments, the present invention provides new methods for preparing a compound having the structure of formula I.

### BRIEF DESCRIPTION OF THE DRAWING

FIG. 1. Thermal ellipsoid drawing of Mo(NAd)(CHCMe₂Ph)(2-CNPyr)₂ (1b) (ellipsoids at 30% probability level). Hydrogen atoms, minor components of disorders, and solvent molecules are omitted for clarity.

FIG. 2. Thermal ellipsoid drawing of 3(PMe₃) (ellipsoids at 50% probability level). Hydrogen atoms and the minor component of the disorder are omitted for clarity. Selected bond lengths (Å) and angles (°) can be found in Table 1.

FIG. 3. Thermal ellipsoid drawing of 2c (ellipsoids at 50% probability level). Hydrogen atoms, minor components of disorders, and the solvent molecule are omitted for clarity. Selected bond lengths (Å) and angles (°) can be found in Table 1.

FIG. 4. Thermal ellipsoid drawing of Mo(NAd)(CHCMe₂Ph)(2-CNPyr)(OHIPT) (4). (ellipsoids at 50% probability level). Hydrogen atoms and minor components of disorders are omitted for clarity. Selected bond lengths (Å) and angles (°) can be found in Table 1.

FIG. 5. Thermal ellipsoid drawing of 5 (ellipsoids at 50% probability level). Hydrogen atoms are omitted for clarity; only one independent molecule is shown. Selected bond lengths (Å) and angles (°) can be found in Table 1.

FIG. 6. The solid state structure of 6d (50% probability ellipsoids). Hydrogen atoms are omitted for clarity. Selected bond lengths (Å) and angles (°) can be found in Table 1.

FIG. 7. The solid state structure of 7b (50% probability ellipsoids). Hydrogen atoms are omitted for clarity, except for the hydrogen on N(2). Selected bond lengths (Å) and angles (°) can be found in Table 1.

FIG. 8. The solid state structure of 8 (50% probability ellipsoids). Hydrogen atoms and minor disorder components are omitted for clarity; only one independent molecule is shown. Selected bond lengths (Å) and angles (°) can be found in Table 1.

FIG. 9. ¹H NMR (bottom) and ¹⁹F NMR (top) spectra of compound B1.

### DETAILED DESCRIPTION OF CERTAIN EMBODIMENTS

### 1. General Description of Certain Embodiments of the Invention

Olefin metathesis is of continuing importance to the synthesis of organic molecules including polymers. The present invention, among other things, provides new compounds for promoting metathesis reactions. In some embodiments, the present invention provides a compound having the structure of formula I:

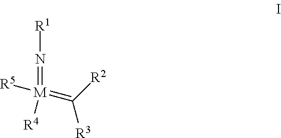

wherein each variable is independently as defined above. Further aspects of compounds of formula I are described in detail, infra.

### 2. Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon, bicyclic hydrocarbon, or tricyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-30 aliphatic carbon atoms. In some embodiments, aliphatic groups contain 1-20 aliphatic carbon atoms. In other embodiments, aliphatic groups contain 1-10 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “cycloaliphatic,” as used herein, refers to saturated or partially unsaturated cyclic aliphatic monocyclic, bicyclic, or polycyclic ring systems, as described herein, having from 3 to 14 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic,” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, that has a single point of attachment to the rest of the molecule, or a C₉-C₁₆ tricyclic hydrocarbon that is completely saturated or that contains one or more units of unsaturation, but which is not aromatic, that has a single point of attachment to the rest of the molecule.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

The term “heteroalkyl” is given its ordinary meaning in the art and refers to alkyl groups as described herein in which one or more carbon atoms is replaced with a heteroatom (e.g., oxygen, nitrogen, sulfur, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “halogen” means F, Cl, Br, or I.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —(CH₂)₀₋₄ SR^{∘}; —(CH₂)₀₋₄S(O)R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄ Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄ SSR^{∘}; —(CH₂)₀₋₄ S(O)₂R^{∘}; —(CH₂)₀₋₄ S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —P(O)(OR^{∘})R^{∘}; —P(O)(OR^{∘})₂; —OP(O)R^{∘}₂; —OP(O)(OR^{∘})R^{∘}; —OP(O)(OR^{∘})₂; —PR^{∘}₂; —P(OR^{∘})R^{∘}; —P(OR^{∘})₂; —OPR^{∘}₂; —OP(OR^{∘})R^{∘}; —OP(OR^{∘})₂; —SiR^{∘}₃; —OSiR^{∘}₃; —SeR^{∘}; —(CH₂)₀₋₄ SeSeR^{∘}; —(C₁₋₄ straight or branched alkylene)O—N(R^{∘})₂; or —(C₁₋₄ straight or branched alkylene)C(O)O—N(R^{∘})₂; wherein each R^{∘} may be substituted as defined below and is independently hydrogen, C₁ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-6 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “stereogenic metal atom” is given its ordinary meaning, and refers to a metal atom coordinated by at least two ligands (e.g., at least four ligands), wherein the ligands are arranged about the metal atom such that the overall structure (e.g., metal complex) lacks a plane of symmetry with respect to the metal atom. In some cases, the stereogenic metal atom may be coordinated by at least three ligands, at least four ligands, at least five ligands, at least six ligands, or more. In certain embodiments, the stereogenic metal atom may be coordinated by four ligands. Metal complexes comprising a stereogenic metal center may provide sufficient space specificity at a reaction site of the metal complex, such that a molecular substrate having a plane of symmetry may be reacted at the reaction site to form a product that is free of a plane of symmetry. That is, the stereogenic metal center of the metal complex may impart sufficient shape specificity to induce stereogenicity effectively, producing a chiral product. Such metal complexes may exhibit improved catalytic activity and stereoselectivity, relative to previous systems, and may reduce undesired side reactions (e.g., dimerization or oligomerization of the metal complex).

The term “chiral” is given its ordinary meaning in the art and refers to a molecule that is not superimposable with its mirror image, wherein the resulting nonsuperimposable mirror images are known as “enantiomers” and are labeled as either an (R) enantiomer or an (S) enantiomer. Typically, chiral molecules lack a plane of symmetry.

The term “achiral” is given its ordinary meaning in the art and refers to a molecule that is superimposable with its mirror image. Typically, achiral molecules possess a plane of symmetry.

As used herein, a ligand may be either monodentate or polydentate. In some embodiments, a ligand is monodentate. In some embodiments, a ligand is bidentate. In some embodiments, a ligand is tridentate. In some embodiments, two or more monodentate ligands are taken together to form a polydentate ligand. A ligand may have hapticity of more than one. In some cases, a ligand has a hapticity of 1 to 10. In some embodiments, a ligand has a hapticity of 1. In some embodiments, a ligand has a hapticity of 2. In some embodiments, a ligand has a hapticity of 3. In some embodiments, a ligand has a hapticity of 4. In some embodiments, a ligand has a hapticity of 5. In some embodiments, a ligand has a hapticity of 6. For a ligand having hapticity greater than one, as sometimes done in the art, a single bond may be drawn between the ligand and the metal. In some cases, a ligand is alkylidene. In some cases, a ligand is a nitrogen-containing ligand. In some cases, a ligand is an oxygen-containing ligand. In some cases, a ligand is a phosphorus-containing ligand. In some embodiments, a ligand comprises an unsaturated bond, and the unsaturated bond is coordinated to a metal. In some embodiments, a ligand comprises a carbon-carbon double bond, and the double bond is coordinated to a metal. In some embodiments, a ligand is an olefin. When an olefin double bond is coordinated to a metal, the chemical bonding between the olefin and the metal can either be depicted as a 3-membered ring wherein the ring members comprises the metal and both carbon atoms of the double bond, or as a single bond between the metal and the double bond.

As used herein, a “nitrogen-containing ligand” may be any species comprising a nitrogen atom. In some cases, the nitrogen atom may bind to the metal atom. In some cases, the nitrogen-containing ligand may bind the metal center via a different atom. In some cases, the nitrogen atom may be a ring atom of a heteroaryl or heteroalkyl group. In some cases, the nitrogen atom may be a substituted amine group. It should be understood that, in catalyst precursors described herein, the nitrogen-containing ligand may have sufficiently ionic character to coordinate a metal center, such as a Mo or W metal center. Examples of nitrogen-containing ligands include, but are not limited to, pyrrolyl, pyrazolyl, pyridinyl, pyrazinyl, pyrimidinyl, imidazolyl, triazolyl, tetrazolyl, oxazolyl, isoxazolyl, thiazolyl, isothiazolyl, indolyl, indazolyl, carbazolyl, morpholinyl, piperidinyl, oxazinyl, substituted derivatives thereof, and the like. For example, the nitrogen-containing ligand may be pyrrolide or 2,5-dimethylpyrrolide. The nitrogen-containing ligand may be selected to interact with an oxygen-containing ligand such that the oxygen-containing ligand can readily replace the nitrogen-containing ligand in a pre-catalyst to generate a catalyst. In cases where the catalyst composition may be generated in situ in order to carry out a chemical reaction, the first, nitrogen-containing ligand may be selected such that, upon replacement by an oxygen-containing ligand, the nitrogen-containing ligands or protonated versions thereof do not interfere with the chemical reaction. In some embodiments, the nitrogen-containing ligand may be chiral and the pre-catalyst may be provided as a racemic mixture or a purified stereoisomer.

In some embodiments, a nitrogen-containing ligand may also describe a ligand precursor comprising at least one hydrogen atom directly bonded to a nitrogen atom, wherein deprotonation of the at least one hydrogen atom results in a negatively charged nitrogen atom, which may coordinate to a metal atom. Exemplary such precursors include but are not limited to amines, amides, and pyrrole and its derivatives thereof. A nitrogen-containing ligand may be a heteroaryl or heteroalkyl group comprising at least one nitrogen ring atom. In some cases, the nitrogen atom may be positioned on a substituent of an alkyl, heteroalkyl, aryl, or heteroaryl group. For example, a nitrogen-containing ligand may be an amine- or amide-substituted aryl group, wherein the amine or amide group is deprotonated upon coordination to the metal center.

As used herein, the term “oxygen-containing ligand” may be used to refer to ligands comprising at least one oxygen atom. In some cases, the oxygen atom binds to the metal atom thereby forming an ether-linkage. In other cases, the oxygen-containing ligand may bind the metal center via a different atom. The term “oxygen-containing ligand” may also describe ligand precursors comprising at least one hydroxyl group (e.g., a hydroxyl-containing ligand), wherein deprotonation of the hydroxyl group results in a negatively charged oxygen atom, which may coordinate to a metal atom. The oxygen-containing ligand may be a heteroaryl or heteroalkyl group comprising at least one oxygen ring atom. In some cases, the oxygen atom may be positioned on a substituent of an alkyl, heteroalkyl, aryl, or heteroaryl group. For example, the oxygen-containing ligand may be a hydroxy-substituted aryl group, wherein the hydroxyl group is deprotonated upon coordination to the metal center.

In some embodiments, an oxygen-containing ligand may also describe a ligand precursor comprising at least one hydroxyl group (e.g., a hydroxyl-containing ligand), wherein deprotonation of the hydroxyl group results in a negatively charged oxygen atom, which may coordinate to a metal atom. An oxygen-containing ligand may be a heteroaryl or heteroalkyl group comprising at least one oxygen ring atom. In some cases, the oxygen atom may be positioned on a substituent of an alkyl, heteroalkyl, aryl, or heteroaryl group. For example, an oxygen-containing ligand may be a hydroxy-substituted aryl group, wherein the hydroxyl group is deprotonated upon coordination to the metal center.

As used herein, the term “phosphorus-containing ligand” may be used to refer to ligands comprising at least one phosphorus atom. In some cases, the phosphorus atom binds to the metal. In other cases, the phosphorus-containing ligand may bind to the metal center via a different atom (i.e., an atom other than the phosphorous). The phosphorus-containing ligand may have phosphorus atom of various oxidation states. In some cases the phosphorus-containing ligand is phosphine. In some cases the phosphorus-containing ligand is phosphite. In some cases the phosphorus-containing ligand is phosphate. The phosphorus-containing ligand may be either monodentate or polydentate. In some cases, two or more phosphorus atoms bind to the metal. In some cases, one or more phosphorus atoms together with one or more non-phosphorus atoms bind to the metal.

As defined herein, a “metal complex” is any complex used to form a provided precursor complex or any complex generated from a provided precursor complex (e.g., for use as a catalyst in a reaction such as a metathesis reaction). In some embodiments, a metal complex is a compound having the structure of formula I described herein.

The phrase “protecting group,” as used herein, refers to temporary substituents which protect a potentially reactive functional group from undesired chemical transformations. Examples of such protecting groups include esters of carboxylic acids, silyl ethers of alcohols, and acetals and ketals of aldehydes and ketones, respectively. A “Si protecting group” is a protecting group comprising a Si atom, such as Si-trialkyl (e.g., trimethylsilyl, tributylsilyl, t-butyldimethylsilyl), Si-triaryl, Si-alkyl-diphenyl (e.g., t-butyldiphenylsilyl), or Si-aryl-dialkyl (e.g., Si-phenyldialkyl). Generally, a Si protecting group is attached to an oxygen atom. The field of protecting group chemistry has been reviewed (Greene, T. W.; Wuts, P. G. M. Protective Groups in Organic Synthesis, 2nd ed.; Wiley: New York, 1991). Such protecting groups (and associated protected moieties) are described in detail below.

Protected hydroxyl groups are well known in the art and include those described in detail in Protecting Groups in Organic Synthesis, T. W. Greene and P. G. M. Wuts, 3rd edition, John Wiley & Sons, 1999, the entirety of which is incorporated herein by reference. Examples of suitably protected hydroxyl groups further include, but are not limited to, esters, carbonates, sulfonates, allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, 0-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention. Such compounds are useful, for example, as analytical tools or probes in biological assays.

As used herein, the term “electron-withdrawing group” is given its ordinary meaning in the art and refers to an atom or group that draws electron density from a neighboring atom or group, usually by resonance and/or inductive effects. In some embodiments, an electron-withdrawing group withdraws electron density from an aromatic ring system by resonance and/or inductive effects. In some embodiments, an electron-withdrawing group withdraws electron density from an aromatic ring system by resonance and inductive effects. In some embodiments, an electron-withdrawing group lowers the electron density of an aromatic ring system such as phenyl. Exemplary electron-withdrawing groups are extensively described in the art, including but not limited to halogen, carbonyl moieties (e.g., aldehyde and ketone groups), —COOH and its derivatives (e.g., ester and amide moieties), protonated amines, quaternary ammonium groups, —CN, —NO₂, —S(O)—, and —S(O)₂—. In some embodiments, an electron-withdrawing group is halogen. In some embodiments, an electron-withdrawing group is —F. In some embodiments, an electron-withdrawing group is —Cl. In some embodiments, an electron-withdrawing group is —Br. In some embodiments, an electron-withdrawing group is —I. In some embodiments, hydrogen is used as reference and regarded as having no effect.

As used herein and in the claims, the singular forms “a”, “an”, and “the” include the plural reference unless the context clearly indicates otherwise. Thus, for example, a reference to “a compound” includes a plurality of such compounds.

### 3. Description of Certain Embodiments of the Invention

In some embodiments, the present invention provides compounds and methods for metathesis reactions. As used herein, the term “metathesis reaction” is given its ordinary meaning in the art and refers to a chemical reaction in which two reacting species exchange partners in the presence of a transition-metal catalyst. In some cases, a byproduct of a metathesis reaction may be ethylene. A metathesis reaction may involve reaction between species comprising, for example, olefins and/or alkynes. Examples of different kinds of metathesis reactions include cross metathesis, ring-closing metathesis, ring-opening metathesis, acyclic diene metathesis, alkyne metathesis, enyne metathesis, ring-opening metathesis polymerization (ROMP), and the like. A metathesis reaction may occur between two substrates which are not joined by a bond (e.g., intermolecular metathesis reaction) or between two portions of a single substrate (e.g., intramolecular metathesis reaction).

In some embodiments, M is molybdenum. In some embodiments, M is tungsten.

As defined generally above, R¹ is an optionally substituted group selected from C₁₋₂₀ aliphatic, C₁₋₂₀ heteroaliphatic having 1-3 heteroatoms independently selected from nitrogen, oxygen, or sulfur, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R¹ is optionally substituted C₁₋₂₀ aliphatic. In some embodiments, R¹ is optionally substituted C₁₋₂₀ cycloaliphatic. In some embodiments, R¹ is optionally substituted C₁₋₁₂ aliphatic. In some embodiments, R¹ is optionally substituted C₁₋₁₂ cycloaliphatic. In some embodiments, R¹ is optionally substituted C₁₋₁₂ cycloalkyl. In some embodiments, R¹ is optionally substituted adamantyl. In some embodiments, R¹ is adamantyl. In some embodiments, R¹ is optionally substituted C₁₋₆ aliphatic. In some embodiments, R¹ is optionally substituted C₁₋₆ alkyl. In some embodiments, R¹ is optionally substituted hexyl, pentyl, butyl, propyl, ethyl or methyl. In some embodiments, R¹ is optionally substituted hexyl. In some embodiments, R¹ is optionally substituted pentyl. In some embodiments, R¹ is optionally substituted butyl. In some embodiments, R¹ is optionally substituted propyl. In some embodiments, R¹ is optionally substituted ethyl. In some embodiments, R¹ is optionally substituted methyl. In some embodiments, R¹ is hexyl. In some embodiments, R¹ is pentyl. In some embodiments, R¹ is butyl. In some embodiments, R¹ is propyl. In some embodiments, R¹ is ethyl. In some embodiments, R¹ is methyl. In some embodiments, R¹ is isopropyl.

In certain embodiments, R¹ is optionally substituted phenyl. In some embodiments, R¹ is substituted phenyl. In some embodiments, R¹ is mono-, di-, tri-, tetra- or penta-substituted phenyl. In some embodiments, R¹ is mono-substituted phenyl. In certain embodiments, R¹ is 2,6-disubstituted phenyl. In some embodiments, R¹ is tri-substituted phenyl. In some embodiments, R¹ is tetra-substituted phenyl. In some embodiments, R¹ is penta-substituted phenyl. In some embodiments, a substituent is a halogen. In some embodiments, a substituent is —F, and R¹ is phenyl substituted with one or more —F. In some embodiments, R¹ is pentafluorophenyl. In some embodiments, a substituent is optionally substituted C₁₋₄ aliphatic. In some embodiments, R¹ is phenyl disubstituted with halogen or C₁₋₄ aliphatic. Such R¹ groups include but are not limited to 2,6-dichlorophenyl, 2,6-dibromophenyl, 2,6-dimethylphenyl, 2,6-di-tert-butylphenyl, and 2,6-diisopropylphenyl.

In some embodiments, R¹ is selected from:

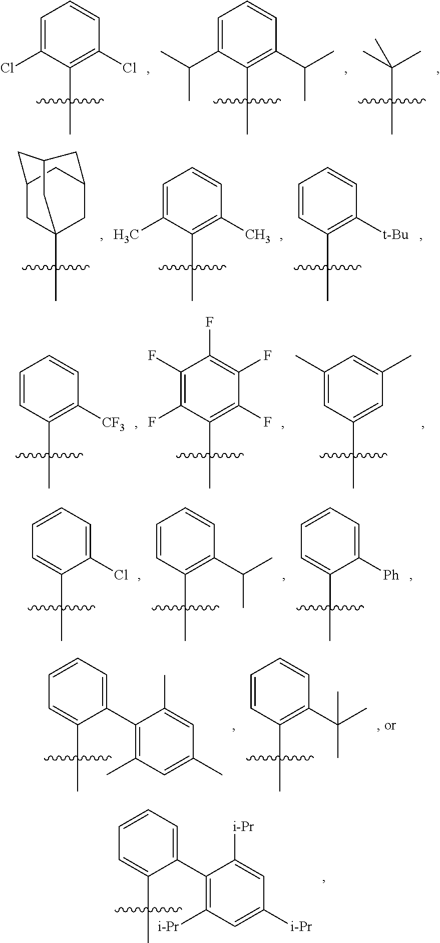

As defined generally above, each of R² and R³ is independently R, —OR, —SR, —N(R)₂, —OC(O)R, —SOR, —SO₂R, —SO₂N(R)₂, —C(O)N(R)₂, —NRC(O)R, or —NRSO₂R, wherein each R is independently as defined above and described herein.

In some embodiments, both of R² and R³ are hydrogen. In some embodiments, one of R² and R³ is hydrogen and the other is an optionally substituted group selected from C₁₋₆ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur, —OR, —SR, —N(R)₂, —OC(O)R, —SOR, —SO₂R, —SO₂N(R)₂, —C(O)N(R)₂, —NRC(O)R, or —NRSO₂R. In some embodiments, one of R² and R³ is hydrogen and the other is an optionally substituted group selected from C₁₋₆ aliphatic, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In certain embodiments, R² or R³ is optionally substituted C₁₋₆ aliphatic. In some embodiments, R² or R³ is optionally substituted C₁₋₆ alkyl. In certain embodiments, R² or R³ is C₁₋₆ alkyl substituted with phenyl and one or two additional substituents. In certain embodiments, R² or R³ is a lower alkyl group optionally substituted with one or two methyl groups and phenyl. In certain embodiments, R² or R³ is —C(Me)₂Ph. In certain embodiments, R² or R³ is —C(Me)₃. In certain embodiments, R² or R³ is —CH═C(Me)Ph.

In some embodiments, each of R² and R³ is independently R, wherein R is as defined above and described herein. In some embodiments, each of R² and R³ is independently R, wherein at least one of R² and R³ is not hydrogen.

In certain embodiments, R² is hydrogen and R³ is R, —OR, —SR, —N(R)₂, —OC(O)R, —SOR, —SO₂R, —SO₂N(R)₂, —C(O)N(R)₂, —NRC(O)R, or —NRSO₂R, wherein each R is independently as defined above and described herein. In certain embodiments, R² is hydrogen and R³ is R, wherein R is as defined above and described herein. In certain embodiments, R² is hydrogen and R³ is optionally substituted C₁₋₂₀ aliphatic. In some embodiments, R² is hydrogen and R³ is optionally substituted C₁₋₂₀ alkyl. In certain embodiments, R² is hydrogen and R³ is C₁₋₆ alkyl substituted with phenyl and one or two additional substituents. In certain embodiments, R² is hydrogen and R³ is a lower alkyl group optionally substituted with one or two methyl groups and phenyl. In certain embodiments, R² is hydrogen and R³ is —C(Me)₂Ph. In certain embodiments, R² is hydrogen and R³ is —C(Me)₃. In certain embodiments, R² is hydrogen and R³ is —CH═C(Me)Ph. In certain embodiments, R² is hydrogen and R³ is —¹³CH═C(Me)Ph. In certain embodiments, R² is hydrogen and R³ is —CH═¹³C(Me)Ph.

As generally defined above, R⁴ is —OR^{s}, wherein R^{s} is as defined above and described herein.

In some embodiments, R⁴ is —O—C(R^{t})₂—R′, wherein each of R^{t} and R′ is independently as defined above and described herein. In some embodiments, R⁴ is —O—C(R^{t})₂—R′, wherein one R^{t} is R. In some embodiments, R⁴ is —O—C(R^{t})₂—R′, wherein one R^{t} is hydrogen. In some embodiments, R⁴ is —O—C(R^{t})₂—R′, wherein one R^{t} is halogen. In some embodiments, R⁴ is —O—C(R^{t})₂—R′, wherein one R^{t} is optionally substituted C₁₋₂₀ aliphatic. In some embodiments, R⁴ is —O—C(R^{t})₂—R′, wherein one R^{t} is optionally substituted C₁₋₆ aliphatic. In some embodiments, R⁴ is —O—C(R^{t})₂—R′, wherein one R^{t} is optionally substituted C₁₋₂₀ haloalkyl. In some embodiments, R⁴ is —O—C(R^{t})₂—R′, wherein R^{t} is optionally substituted C₁₋₆ aliphatic comprising one or more —F. In some embodiments, R⁴ is —O—C(R^{t})₂—R′, wherein one R^{t} is —CF₃. In some embodiments, R⁴ is —O—C(R^{t})₂—R′, wherein each R^{t} is —CF₃.

In some embodiments, R⁴ is —O—C(R^{t})₂—R′, wherein R′ is hydrogen. In some embodiments, R⁴ is —O—C(R^{t})₂, and each R^{t} is R. In some embodiments, R⁴ is —O—CH(R)₂, wherein the two R groups are taken together with the carbon atom to which they are attached to form an optionally substituted 3-10 membered, monocyclic or bicyclic, saturated, or partially unsaturated ring having 0-4 heteroatoms independently selected from nitrogen, oxygen, or sulfur.

In some embodiments, R⁴ is —O—C(R^{t})₂—R′, wherein R′ is optionally substituted C₁₋₂₀ aliphatic. In some embodiments, R⁴ is —O—C(R^{t})₂—R′, wherein R′ is optionally substituted C₁₋₆ aliphatic comprising one or more —F. In some embodiments, R⁴ is —O—C(R^{t})₂—R′, wherein R′ is optionally substituted C₁₋₄ aliphatic comprising one or more —F.

In some embodiments, R⁴ is hexafluoro-tert-butoxide (—OCMe(CF₃)₂, OR_{F6}). In some embodiments, R⁴ is perfluoro-tert-butoxide (—OC(CF₃)₃, (OR_{F9}). In some embodiments, R⁴ is 1,1,1,3,3,3-hexafluoroisopropoxide (O-iPr^{F6}).

In some embodiments, R⁴ is —OR″. In some embodiments, R⁴ is —OR″, wherein R″ is optionally substituted phenyl. In some embodiments, R⁴ is 2,6-bis(2′,4′,6′-triisopropylphenyl)phen-2-oxide (HIPTO). In some embodiments, R⁴ is 2,6-bis(2′,4′,6′-trimethylphenyl)phenoxide (HMTO). In some embodiments, R⁴ is pentafluorophenoxide (—OC₆F₅). In some embodiments, R⁴ is 2,6-diisopropylphenoxide. In some embodiments, R⁴ is 4-dimethylamino-2,6-diphenylphenoxide. In some embodiments, R⁴ is 2,6-dimethoxylphenoxide.

In some embodiments, R⁴ is —OR^{s}, wherein R^{s} is an optionally substituted 3-7 membered saturated or partially unsaturated carbocyclic ring. In some embodiments, R⁴ is —OR^{s}, wherein R^{s} is an optionally substituted 8-10 membered bicyclic saturated, partially unsaturated or aryl ring. In some embodiments, R⁴ is —OR^{s}, wherein R^{s} is an optionally substituted 5-6 membered monocyclic heteroaryl ring having 1-4 heteroatoms independently selected from nitrogen, oxygen, or sulfur. In some embodiments, R⁴ is —OR^{s}, wherein R^{s} is an optionally substituted 3-7 membered saturated or partially unsaturated heterocyclic ring having 1-3 heteroatoms independently selected from nitrogen, oxygen, or sulfur. In some embodiments, R⁴ is —OR^{s}, wherein R^{s} is an optionally substituted 7-10 membered bicyclic saturated or partially unsaturated heterocyclic ring having 1-5 heteroatoms independently selected from nitrogen, oxygen, or sulfur. In some embodiments, R⁴ is —OR^{s}, wherein R^{s} is an optionally substituted 8-10 membered bicyclic heteroaryl ring having 1-5 heteroatoms independently selected from nitrogen, oxygen, or sulfur.

In some embodiments, R⁴ is —OAr^{a}, wherein Ar^{a} is as defined above and described herein. In some embodiments, R⁴ is 2,6-bis(2′,4′,6′-triisopropylphenyl)phen-2-oxide (HIPTO). In some embodiments, R⁴ is 2,6-bis(2′,4′,6′-trimethylphenyl)phenoxide (HMTO).

As generally defined above, R⁵ is different from —R⁴, and is —OR′, —OC(O)R′, —N(R′)₂, or R″, wherein each of R′ and R″ is independently as defined above and described herein.

As generally defined above, R^{s} is —C(R^{t})₂—R′, —Ar^{a}, or an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

As generally defined above, R^{s} is —C(R^{t})₂—R′, —Ar^{a}, or an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R^{s} is —C(R^{t})₂—R′. In some embodiments, R^{s} is —C(R^{t})₂—R′, wherein one R^{t} is R. In some embodiments, R^{s} is —C(R^{t})₂—R′, wherein one R^{t} is hydrogen. In some embodiments, R^{s} is —C(R^{t})₂—R′, wherein one R^{t} is halogen. In some embodiments, R^{s} is —C(R^{t})₂—R′, wherein one R^{t} is optionally substituted C₁₋₂₀ aliphatic. In some embodiments, R^{s} is —C(R^{t})₂—R′, wherein one R^{t} is optionally substituted C₁₋₆ aliphatic. In some embodiments, R^{s} is —C(R^{t})₂—R′, wherein one R^{t} is optionally substituted C₁₋₆ alkyl. In some embodiments, R^{s} is —C(R^{t})₂—R′, wherein one R^{t} is optionally substituted C₁₋₆ methyl. In some embodiments, R^{s} is —C(R^{t})₂—R′, wherein one R^{t} is optionally substituted C₁₋₂₀ haloalkyl. In some embodiments, R^{s} is —C(R^{t})₂—R′, wherein one R^{t} is optionally substituted C₁₋₆ aliphatic comprising one or more —F. In some embodiments, R^{s} is —C(R^{t})₂—R′, wherein one R^{t} is —CF₃.

In some embodiments, R^{s} is —C(R^{t})₂—R′. In some embodiments, R^{s} is —C(R^{t})₂—R′, wherein each R^{t} is R. In some embodiments, R^{s} is —C(R^{t})₂—R′, wherein each R^{t} is hydrogen. In some embodiments, R^{s} is —C(R^{t})₂—R′, wherein each R^{t} is halogen. In some embodiments, R^{s} is —C(R^{t})₂—R′, wherein each R^{t} is optionally substituted C₁₋₂₀ aliphatic. In some embodiments, R^{s} is —C(R^{t})₂—R′, wherein each R^{t} is optionally substituted C₁₋₆ aliphatic. In some embodiments, R^{s} is —C(R^{t})₂—R′, wherein each R^{t} is optionally substituted C₁₋₆ alkyl. In some embodiments, R^{s} is —C(R^{t})₂—R′, wherein each R^{t} is optionally substituted C₁₋₆ methyl. In some embodiments, R^{s} is —C(R^{t})₂—R′, wherein each R^{t} is optionally substituted C₁₋₂₀ haloalkyl. In some embodiments, R^{s} is —C(R^{t})₂—R′, wherein each R^{t} is optionally substituted C₁₋₆ aliphatic comprising each or more —F. In some embodiments, R^{s} is —C(R^{t})₂—R′, wherein each R^{t} is —CF₃.

In some embodiments, R^{s} is —C(R^{t})₂—R′, wherein R′ is hydrogen. In some embodiments, R^{s} is —C(R^{t})₂, and each R^{t} is R. In some embodiments, R^{s} is —CH(R)₂, wherein the two R groups are taken together with the carbon atom to which they are attached to form an optionally substituted 3-10 membered, monocyclic or bicyclic, saturated, or partially unsaturated ring having 0-4 heteroatoms independently selected from nitrogen, oxygen, or sulfur.

In some embodiments, R^{s} is —C(R^{t})₂—R′, wherein R′ is optionally substituted C₁₋₂₀ aliphatic. In some embodiments, R^{s} is —C(R^{t})₂—R′, wherein R′ is optionally substituted C₁₋₆ aliphatic comprising one or more —F. In some embodiments, R⁴ is —C(R^{t})₂—R′, wherein R′ is optionally substituted C₁₋₄ aliphatic comprising one or more —F.

In some embodiments, R^{s} is R″. In some embodiments, R^{s} is —Ar^{a}.

In some embodiments, R^{s} is an optionally substituted 3-7 membered saturated or partially unsaturated carbocyclic ring. In some embodiments, R^{s} is an optionally substituted 8-10 membered bicyclic saturated, partially unsaturated or aryl ring. In some embodiments, R^{s} is an optionally substituted 5-6 membered monocyclic heteroaryl ring having 1-4 heteroatoms independently selected from nitrogen, oxygen, or sulfur. In some embodiments, R^{s} is an optionally substituted 3-7 membered saturated or partially unsaturated heterocyclic ring having 1-3 heteroatoms independently selected from nitrogen, oxygen, or sulfur. In some embodiments, R^{s} is an optionally substituted 7-10 membered bicyclic saturated or partially unsaturated heterocyclic ring having 1-5 heteroatoms independently selected from nitrogen, oxygen, or sulfur. In some embodiments, R^{s} is an optionally substituted 8-10 membered bicyclic heteroaryl ring having 1-5 heteroatoms independently selected from nitrogen, oxygen, or sulfur.

As generally defined above, each R^{t} is independently halogen or R. In some embodiments, R^{t} is halogen. In some embodiments, R^{t} is —F. In some embodiments, R^{t} is —Cl. In some embodiments, R^{t} is —Br. In some embodiments, R^{t} is —I.

In some embodiments, R^{t} is R, wherein R is as defined above and described herein. In some embodiments, R^{t} is hydrogen.

In some embodiments, R⁵ is different from R⁴ and is —OR′. In some embodiments, R⁵ is different from R⁴ and is an alkoxy or aryloxy group having the structure of —OR′. In some embodiments, R⁵ is —OR′, wherein R is not hydrogen. In some embodiments, R⁵ is —OAr^{a}, wherein Ar^{a} is as defined above and described herein. In some embodiments, R⁵ is —OR′, wherein R′ is optionally substituted C₁₋₂₀ aliphatic. In some embodiments, R⁵ is —OR′, wherein R′ is optionally substituted C₁₋₆ aliphatic comprising one or more —F. In some embodiments, R⁵ is —OR′, wherein R′ is optionally substituted C₁₋₄ aliphatic comprising one or more —F. In some embodiments, R⁵ is hexafluoro-tert-butoxide, (—OCMe(CF₃)₂, OR_{F6}). In some embodiments, R⁵ is perfluoro-tert-butoxide (—OC(CF₃)₃, OR_{F9}). In some embodiments, R⁴ is 1,1,1,3,3,3-hexafluoroisopropoxide (O-iPr^{F6}). In some embodiments, R⁵ is —OR′, wherein R′ is R″. In some embodiments, R⁵ is —OR′, wherein R′ is optionally substituted phenyl. In some embodiments, R⁵ is 2,6-bis(2′,4′,6′-triisopropylphenyl)phen-2-oxide (HIPTO). In some embodiments, R⁵ is 2,6-bis(2′,4′,6′-trimethylphenyl)phenoxide (HMTO). In some embodiments, R⁵ is 2,2′,6,6′-tetraisopropylterphen-2-oxide (TIPTO). In some embodiments, R⁵ is pentafluorophenoxide (—OC₆F₅). In some embodiments, R⁵ is 2,6-diisopropylphenoxide. In some embodiments, R⁵ is 4-dimethylamino-2,6-diphenylphenoxide. In some embodiments, R⁵ is 2,6-dimethoxylphenoxide.

In some embodiments, R⁵ is —OC(O)R′, wherein R′ is as defined above and described herein. In some embodiments, R⁵ is —OC(O)R′, wherein R′ is R″.

In some embodiments, R⁵ is —N(R′)₂, wherein each R′ independently as defined above and described herein. In some embodiments, R⁵ is —NHR′. In some embodiments, R⁵ is —NHR″.

In some embodiments, R⁵ is R″, wherein R″ is as defined above and described herein. In some embodiments, R⁵ is R″ and is bonded to M through an aromatic carbon atom. In some embodiments, R⁵ is 2,6-bis(2′,4′,6′-trimethylphenyl)phenyl-2-amide (HMT(H)N).

As generally defined above, R′ is hydrogen —Ar^{a}, or an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R′ is hydrogen. In some embodiments, R′ is not hydrogen.

In some embodiments, R′ is —Ar^{a}, an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R′ is —Ar^{a}, wherein Ar^{a} is as defined above and described herein.

In some embodiments, R is optionally substituted C₁₋₂₀ aliphatic. In some embodiments, R′ is optionally substituted C₁₋₁₅ aliphatic. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is optionally substituted hexyl, pentyl, butyl, propyl, ethyl or methyl. In some embodiments, R′ is optionally substituted hexyl. In some embodiments, R′ is optionally substituted pentyl. In some embodiments, R′ is optionally substituted butyl. In some embodiments, R′ is optionally substituted propyl. In some embodiments, R′ is optionally substituted ethyl. In some embodiments, R′ is optionally substituted methyl. In some embodiments, R′ is hexyl. In some embodiments, R′ is pentyl. In some embodiments, R′ is butyl. In some embodiments, R′ is propyl. In some embodiments, R′ is ethyl. In some embodiments, R′ is methyl. In some embodiments, R′ is isopropyl. In some embodiments, R′ is n-propyl. In some embodiments, R′ is tert-butyl. In some embodiments, R′ is sec-butyl. In some embodiments, R′ is n-butyl.

In some embodiments, R′ is optionally substituted C₁₋₂₀ aliphatic. In some embodiments, R′ is optionally substituted C₁₋₆ aliphatic comprising one or more —F. In some embodiments, R′ is optionally substituted C₁₋₄ aliphatic comprising one or more —F. In some embodiments, R′ is —CMe(CF₃)₂. In some embodiments, R′ is —C(CF₃)₃. In some embodiments, R′ is 1,1,1,3,3,3-hexafluoroisopropyl.

In some embodiments, R′ is optionally substituted phenyl. In some embodiments, R′ is 2,6-terphenyl (Ter). In some embodiments, R′ is 2,6-bis(4′-methylphenyl)phenyl (Ter_{Me}). In some embodiments, R′ is 2,6-bis(4′-methoxyphenyl)phenyl (Ter_{OMe}). In some embodiments, R′ is 2,2′,6,6′-tetraisopropylterphenyl (TIPT). In some embodiments, R′ is 2,4,6-triisopropylphenyl (TRIP). In some embodiments, R′ is pentafluorophenyl (—C₆F₅). In some embodiments, R′ is 2,6-diisopropylphenyl. In some embodiments, R′ is 4-dimethylamino-2,6-diphenylphenyl. In some embodiments, R′ is 2,6-dimethoxylphenyl. In some embodiments, R′ is 2,6-diisopropylphenyl (Ar). In some embodiments, R′ is 2,6-dimethylphenyl (Ar′). In some embodiments, R′ is 2-(trifluoromethyl)phenyl (Ar^{CF3}). In some embodiments, R′ is 2-chlorophenyl (Ar^{Cl}). In some embodiments, R′ is 2-isopropylphenyl (Ar^{iPr}). In some embodiments, R′ is 2-biphenyl (Ar^{Ph}). In some embodiments, R′ is 3,5-dimethylphenyl (Ar^{m}). In some embodiments, R′ is 2-(2′,4′,6′-trimethylphenyl)phenyl (Ar^{m}). In some embodiments, R′ is 2-tert-butylphenyl (Ar^{tBu}). In some embodiments, R′ is 2-(2′,4′,6′-triisopropylphenyl)phenyl (Ar^{T}). In some embodiments, R′ is 2,6-bis(2′,4′,6′-triisopropylphenyl)phenyl (HIPT). In some embodiments, R′ is 2,6-bis(2′,4′,6′-trimethylphenyl)phenyl (HMT). In some embodiments, R′ is mesityl. In some embodiments, R′ is phenyl.

In some embodiments, R′ is optionally substituted phenyl wherein one or more substituents are halogen. In some embodiments, R′ is optionally substituted phenyl wherein one or more substituents are —F. In some embodiments, R′ is optionally substituted phenyl wherein one or more substituents are —Cl. In some embodiments, R′ is optionally substituted phenyl wherein one or more substituents are —Br. In some embodiments, R′ is optionally substituted phenyl wherein one or more substituents are —I.

In some embodiments, R′ is optionally substituted phenyl wherein one or more substituents are C₁₋₆ aliphatic. In some embodiments, R′ is optionally substituted phenyl wherein one or more substituents are C₁₋₆ phenyl. In some embodiments, R′ is 2,6-diisopropylphenyl (Ar). In some embodiments, R′ is 2,6-dimethylphenyl (Ar′). In some embodiments, R′ is 2-(trifluoromethyl)phenyl (Ar^{CF3}). In some embodiments, R′ is 2-chlorophenyl (Ar^{Cl}). In some embodiments, R′ is 2-isopropylphenyl (Ar^{iPr}). In some embodiments, R′ is 3,5-dimethylphenyl (Ar^{m}). In some embodiments, R′ is 2-tert-butylphenyl (Ar^{tBu}).

In some embodiments, R′ is an optionally substituted 3-7 membered saturated or partially unsaturated carbocyclic ring. In some embodiments, R′ is an optionally substituted 3-membered saturated or partially unsaturated carbocyclic ring. In some embodiments, R′ is an optionally substituted 4-membered saturated or partially unsaturated carbocyclic ring. In some embodiments, R′ is an optionally substituted 5-membered saturated or partially unsaturated carbocyclic ring. In some embodiments, R′ is an optionally substituted 6-membered saturated or partially unsaturated carbocyclic ring. In some embodiments, R′ is an optionally substituted 7-membered saturated or partially unsaturated carbocyclic ring. In some embodiments, R′ is an optionally substituted cycloheptyl. In some embodiments, R′ is an optionally substituted cyclohexyl. In some embodiments, R′ is an optionally substituted cyclopentyl. In some embodiments, R′ is an optionally substituted cyclobutyl. In some embodiments, R′ is an optionally substituted cyclopropyl.

In some embodiments, R′ is an optionally substituted 8-10 membered bicyclic saturated, partially unsaturated or aryl ring. In some embodiments, R′ is an optionally substituted 8-10 membered bicyclic saturated ring. In some embodiments, R′ is an optionally substituted 8-10 membered bicyclic partially unsaturated ring. In some embodiments, R′ is an optionally substituted 8-10 membered bicyclic aryl ring. In some embodiments, R′ is optionally substituted naphthyl.

In some embodiments, R′ is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur. In some embodiments, R′ is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is optionally substituted phenyl. In some embodiments, R′ is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is an optionally substituted 5-6 membered monocyclic heteroaryl ring having 1-4 heteroatoms independently selected from nitrogen, oxygen, or sulfur. In some embodiments, R′ is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is an optionally substituted 8-10 membered bicyclic aryl ring. In some embodiments, R′ is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is optionally substituted naphthyl. In some embodiments, R′ is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is an optionally substituted 8-10 membered bicyclic heteroaryl ring having 1-5 heteroatoms independently selected from nitrogen, oxygen, or sulfur. In some embodiments, R′ is optionally substituted biaryl wherein each aryl group is independently optionally substituted phenyl. In some embodiments, R′ is optionally substituted biaryl wherein each aryl group is independently optionally substituted phenyl, or an optionally substituted 5-6 membered monocyclic heteroaryl ring having 1-4 heteroatoms independently selected from nitrogen, oxygen. In some embodiments, R′ is optionally substituted biaryl wherein each aryl group is independently an optionally substituted 8-10 membered bicyclic aryl ring. In some embodiments, R′ is optionally substituted biaryl wherein one aryl group is optionally substituted naphthyl, and the other aryl group is independently an optionally substituted 8-10 membered bicyclic aryl ring. In some embodiments, R′ is optionally substituted biaryl wherein each aryl group is optionally substituted naphthyl. In some embodiments, R′ is optionally substituted biaryl wherein one aryl group is optionally substituted naphthyl, and the other aryl group is an optionally substituted 8-10 membered bicyclic heteroaryl ring having 1-5 heteroatoms independently selected from nitrogen, oxygen, or sulfur.

In some embodiments, R′ is an optionally substituted 5-6 membered monocyclic heteroaryl ring having 1-4 heteroatoms independently selected from nitrogen, oxygen, or sulfur. In some embodiments, R′ is a substituted 5-6 membered monocyclic heteroaryl ring having 1-4 heteroatoms independently selected from nitrogen, oxygen, or sulfur. In some embodiments, R′ is an unsubstituted 5-6 membered monocyclic heteroaryl ring having 1-4 heteroatoms independently selected from nitrogen, oxygen, or sulfur.

In some embodiments, R′ is an optionally substituted 5-membered monocyclic heteroaryl ring having 1-4 heteroatoms independently selected from nitrogen, oxygen or sulfur. In some embodiments, R′ is an optionally substituted 6-membered monocyclic heteroaryl ring having 1-4 heteroatoms independently selected from nitrogen, oxygen, or sulfur.

In some embodiments, R′ is an optionally substituted 5-membered monocyclic heteroaryl ring having one heteroatom selected from nitrogen, oxygen, or sulfur. In some embodiments, R′ is selected from optionally substituted pyrrolyl, furanyl, or thienyl.

In some embodiments, R′ is an optionally substituted 5-membered heteroaryl ring having two heteroatoms independently selected from nitrogen, oxygen, or sulfur. In certain embodiments, R′ is an optionally substituted 5-membered heteroaryl ring having one nitrogen atom, and an additional heteroatom selected from sulfur or oxygen. Exemplary R′ groups include but are not limited to optionally substituted pyrazolyl, imidazolyl, thiazolyl, isothiazolyl, oxazolyl or isoxazolyl.

In some embodiments, R′ is an optionally substituted 5-membered heteroaryl ring having three heteroatoms independently selected from nitrogen, oxygen, or sulfur. Exemplary R′ groups include but are not limited to optionally substituted triazolyl, oxadiazolyl or thiadiazolyl.

In some embodiments, R′ is an optionally substituted 5-membered heteroaryl ring having four heteroatoms independently selected from nitrogen, oxygen, or sulfur. Exemplary R′ groups include but are not limited to optionally substituted tetrazolyl, oxatriazolyl and thiatriazolyl.

In some embodiments, R′ is an optionally substituted 6-membered heteroaryl ring having 1-4 nitrogen atoms. In some embodiments, R′ is an optionally substituted 6-membered heteroaryl ring having 1-3 nitrogen atoms. In other embodiments, R′ is an optionally substituted 6-membered heteroaryl ring having 1-2 nitrogen atoms. In some embodiments, R′ is an optionally substituted 6-membered heteroaryl ring having four nitrogen atoms. In some embodiments, R′ is an optionally substituted 6-membered heteroaryl ring having three nitrogen atoms. In some embodiments, R′ is an optionally substituted 6-membered heteroaryl ring having two nitrogen atoms. In certain embodiments, R′ is an optionally substituted 6-membered heteroaryl ring having one nitrogen atom. Exemplary R′ groups include but are not limited to optionally substituted pyridinyl, pyrimidinyl, pyrazinyl, pyridazinyl, triazinyl, or tetrazinyl.

In some embodiments, R′ is an optionally substituted 3-7 membered saturated or partially unsaturated heterocyclic ring having 1-3 heteroatoms independently selected from nitrogen, oxygen, or sulfur. In some embodiments, R′ is a substituted 3-7 membered saturated or partially unsaturated heterocyclic ring having 1-3 heteroatoms independently selected from nitrogen, oxygen, or sulfur. In some embodiments, R′ is an unsubstituted 3-7 membered saturated or partially unsaturated heterocyclic ring having 1-3 heteroatoms independently selected from nitrogen, oxygen, or sulfur.

In certain embodiments, R′ is an optionally substituted 5-7 membered partially unsaturated monocyclic ring having 1-3 heteroatoms independently selected from nitrogen, oxygen, or sulfur. In certain embodiments, R′ is an optionally substituted 5-6 membered partially unsaturated monocyclic ring having 1-3 heteroatoms independently selected from nitrogen, oxygen, or sulfur. In certain embodiments, R′ is an optionally substituted 5-membered partially unsaturated monocyclic ring having 1-3 heteroatoms independently selected from nitrogen, oxygen, or sulfur. Exemplary R′ groups include but are not limited to optionally substituted dihydroimidazolyl, dihydrothiazolyl, dihydrooxazolyl, or oxazolinyl. In certain embodiments, R′ is an optionally substituted 6-membered partially unsaturated monocyclic ring having 1-3 heteroatoms independently selected from nitrogen, oxygen, or sulfur. Exemplary R′ groups include but are not limited to optionally substituted dihydropyridinyl, tetrahydropyridinyl, dihydropyrimidinyl, tetrahydropyrimidinyl, dihydropyrazinyl, tetrohydropyrazinyl, dihydrotriazinyl, tetrahydrotriazinyl, dihydrodioxinyl, dihydrooxathiinyl, dihydrooxazinyl, dihydrodithiine, dihydrothiazine, dioxinyl, oxathiinyl, oxazinyl, dithiinyl, or thiazinyl. In certain embodiments, R′ is an optionally substituted 7-membered partially unsaturated monocyclic ring having 1-3 heteroatoms independently selected from nitrogen, oxygen, or sulfur. Exemplary R′ groups include but are not limited to optionally substituted azepiyl, oxepinyl, thiepinyl, diazepinyl, oxazepinyl, thiazepinyl, triazepinyl, oxadiazepinyl, thiadiazepinyl, dihydroazepiyl, dihydrooxepinyl, dihydrothiepinyl, dihydrodiazepinyl, dihydrooxazepinyl, dihydrothiazepinyl, dihydrotriazepinyl, dihydrooxadiazepinyl, dihydrothiadiazepinyl, tetrahydroazepiyl, tetrahydrooxepinyl, tetrahydrothiepinyl, tetrahydrodiazepinyl, tetrahydrooxazepinyl, tetrahydrothiazepinyl, tetrahydrotriazepinyl, tetrahydrooxadiazepinyl, or tetrahydrothiadiazepinyl.

In some embodiments, R′ is an optionally substituted 3-membered heterocyclic ring having one heteroatom selected from nitrogen, oxygen or sulfur. Exemplary R′ groups include but are not limited to optionally substituted aziridinyl, thiiranyl or oxiranyl. In some embodiments, R′ is optionally substituted 4-membered heterocyclic ring having 1-3 heteroatoms independently selected from nitrogen, oxygen, or sulfur. Exemplary R′ groups include but are not limited to optionally substituted azetidinyl, oxetanyl, thietanyl, oxazetidinyl, thiazetidinyl, or diazetidinyl. In some embodiments, R′ is optionally substituted 5-membered heterocyclic ring having 1-3 heteroatoms independently selected from nitrogen, oxygen, or sulfur. Exemplary R′ groups include but are not limited to optionally substituted pyrrolidinyl, tetrahydrofuranyl, tetrahydrothienyl, oxazolidinyl, dioxolanyl, oxathiolanyl, thiazolidinyl, dithiolanyl, imidazolidinyl, isothiazolidinyl, pyrazolidinyl, isoxazolidinyl, isothiazolidinyl, triazolidinyl, oxadiazolidinyl, thiadiazolidinyl, oxadiazolidinyl, dioxazolidinyl, oxathiazolidinyl, thiadiazolidinyl or dithiazolidinyl. In some embodiments, R′ is optionally substituted 6-membered heterocyclic ring having 1-3 heteroatoms independently selected from nitrogen, oxygen, or sulfur. Exemplary R′ groups include but are not limited to optionally substituted piperidinyl, tetrahydropyranyl, tetrahydrothiopyranyl, piperazinyl, morpholinyl, thiomorpholinyl, dithianyl, dioxanyl, oxathianyl, triazinanyl, oxadiazinanyl, thiadiazinanyl, dithiazinanyl, dioxazinanyl, oxathiazinanyl, oxadithianyl, trioxanyl, dioxathianyl or trithianyl. In some embodiments, R′ is optionally substituted 7-membered heterocyclic ring having 1-3 heteroatoms independently selected from nitrogen, oxygen, or sulfur. Exemplary R′ groups include but are not limited to optionally substituted azepanyl, oxepanyl, thiepanyl, diazepanyl, oxazepanyl, thiazepanyl, dioxepanyl, oxathiepanyl, dithiepanyl, triazepanyl, oxadiazepanyl, thiadiazepanyl, dioxazepanyl, oxathiazepanyl, dithiazepanyl, trioxepanyl, dioxathiepanyl, oxadithiepanyl or trithiepanyl.

In certain embodiments, R′ is optionally substituted oxiranyl, oxetanyl, tetrahydrofuranyl, tetrahydropyranyl, oxepaneyl, aziridineyl, azetidineyl, pyrrolidinyl, piperidinyl, azepanyl, thiiranyl, thietanyl, tetrahydrothienyl, tetrahydrothiopyranyl, thiepanyl, dioxolanyl, oxathiolanyl, oxazolidinyl, imidazolidinyl, thiazolidinyl, dithiolanyl, dioxanyl, morpholinyl, oxathianyl, piperazinyl, thiomorpholinyl, dithianyl, dioxepanyl, oxazepanyl, oxathiepanyl, dithiepanyl, diazepanyl, dihydrofuranonyl, tetrahydropyranonyl, oxepanonyl, pyrolidinonyl, piperidinonyl, azepanonyl, dihydrothiophenonyl, tetrahydrothiopyranonyl, thiepanonyl, oxazolidinonyl, oxazinanonyl, oxazepanonyl, dioxolanonyl, dioxanonyl, dioxepanonyl, oxathiolinonyl, oxathianonyl, oxathiepanonyl, thiazolidinonyl, thiazinanonyl, thiazepanonyl, imidazolidinonyl, tetrahydropyrimidinonyl, diazepanonyl, imidazolidinedionyl, oxazolidinedionyl, thiazolidinedionyl, dioxolanedionyl, oxathiolanedionyl, piperazinedionyl, morpholinedionyl, thiomorpholinedionyl, tetrahydropyranyl, tetrahydrofuranyl, morpholinyl, thiomorpholinyl, piperidinyl, piperazinyl, pyrrolidinyl, tetrahydrothienyl, or tetrahydrothiopyranyl.

In some embodiments, R′ is an optionally substituted 7-10 membered bicyclic saturated or partially unsaturated heterocyclic ring having 1-5 heteroatoms independently selected from nitrogen, oxygen, or sulfur. In some embodiments, R′ is optionally substituted indolinyl. In some embodiments, R′ is optionally substituted isoindolinyl. In some embodiments, R′ is optionally substituted 1,2,3,4-tetrahydroquinolinyl. In some embodiments, R′ is optionally substituted 1,2,3,4-tetrahydroisoquinolinyl. In some embodiments, R′ is an optionally substituted azabicyclo[3.2.1]octanyl.

In some embodiments, R is an optionally substituted 8-10 membered bicyclic heteroaryl ring having 1-5 heteroatoms independently selected from nitrogen, oxygen, or sulfur.

In some embodiments, R′ is an optionally substituted 5,6-fused heteroaryl ring having 1-5 heteroatoms independently selected from nitrogen, oxygen, or sulfur. In some embodiments, R′ is an optionally substituted 5,6-fused heteroaryl ring having 1-4 heteroatoms independently selected from nitrogen, oxygen, or sulfur. In some embodiments, R′ is an optionally substituted 5,6-fused heteroaryl ring having 1-3 heteroatoms independently selected from nitrogen, oxygen, or sulfur. In some embodiments, R′ is an optionally substituted 5,6-fused heteroaryl ring having two heteroatoms independently selected from nitrogen, oxygen, or sulfur. In some embodiments, R′ is optionally substituted 1,4-dihydropyrrolo[3,2-b]pyrrolyl, 4H-furo[3,2-b]pyrrolyl, 4H-thieno[3,2-b]pyrrolyl, furo[3,2-b]furanyl, thieno[3,2-b]furanyl, thieno[3,2-b]thienyl, 1H-pyrrolo[ 1,2-a]imidazolyl, pyrrolo[2,1-b]oxazolyl or pyrrolo[2,1-b]thiazolyl. In some embodiments, R′ is an optionally substituted 5,6-fused heteroaryl ring having three heteroatoms independently selected from nitrogen, oxygen, or sulfur. In some embodiments, R′ is optionally substituted dihydropyrroloimidazolyl, 1H-furoimidazolyl, 1H-thienoimidazolyl, furooxazolyl, furoisoxazolyl, 4H-pyrrolooxazolyl, 4H-pyrroloisoxazolyl, thienooxazolyl, thienoisoxazolyl, 4H-pyrrolothiazolyl, furothiazolyl, thienothiazolyl, 1H-imidazoimidazolyl, imidazooxazolyl or imidazo[5,1-b]thiazolyl. In some embodiments, R′ is an optionally substituted 5,6-fused heteroaryl ring having four heteroatoms independently selected from nitrogen, oxygen, or sulfur. In some embodiments, R′ is an optionally substituted 5,6-fused heteroaryl ring having five heteroatoms independently selected from nitrogen, oxygen, or sulfur.

In some embodiments, R′ is an optionally substituted 5,6-fused heteroaryl ring having 1-5 heteroatoms independently selected from nitrogen, oxygen, or sulfur. In other embodiments, R′ is an optionally substituted 5,6-fused heteroaryl ring having 1-2 heteroatoms independently selected from nitrogen, oxygen, or sulfur. In certain embodiments, R′ is an optionally substituted 5,6-fused heteroaryl ring having one heteroatom independently selected from nitrogen, oxygen, or sulfur. In some embodiments, R′ is optionally substituted indolyl. In some embodiments, R′ is optionally substituted benzofuranyl. In some embodiments, R′ is optionally substituted benzo[b]thienyl. In certain embodiments, R′ is an optionally substituted 5,6-fused heteroaryl ring having two heteroatoms independently selected from nitrogen, oxygen, or sulfur. In some embodiments, R′ is optionally substituted azaindolyl. In some embodiments, R′ is optionally substituted benzimidazolyl. In some embodiments, R′ is optionally substituted benzothiazolyl. In some embodiments, R′ is optionally substituted benzoxazolyl. In some embodiments, R′ is an optionally substituted indazolyl. In certain embodiments, R′ is an optionally substituted 5,6-fused heteroaryl ring having three heteroatoms independently selected from nitrogen, oxygen, or sulfur. In some embodiments, R′ is optionally substituted oxazolopyridiyl, thiazolopyridinyl or imidazopyridinyl. In certain embodiments, R′ is an optionally substituted 5,6-fused heteroaryl ring having four heteroatoms independently selected from nitrogen, oxygen, or sulfur. In some embodiments, R′ is optionally substituted purinyl, oxazolopyrimidinyl, thiazolopyrimidinyl, oxazolopyrazinyl, thiazolopyrazinyl, imidazopyrazinyl, oxazolopyridazinyl, thiazolopyridazinyl or imidazopyridazinyl. In certain embodiments, R′ is an optionally substituted 5,6-fused heteroaryl ring having five heteroatoms independently selected from nitrogen, oxygen, or sulfur.

In certain embodiments, R′ is an optionally substituted 6,6-fused heteroaryl ring having 1-5 heteroatoms independently selected from nitrogen, oxygen, or sulfur. In some embodiments, R′ is an optionally substituted 6,6-fused heteroaryl ring having 1-2 heteroatoms independently selected from nitrogen, oxygen, or sulfur. In other embodiments, R′ is an optionally substituted 6,6-fused heteroaryl ring having one heteroatom selected from nitrogen, oxygen, or sulfur. In some embodiments, R′ is optionally substituted quinolinyl. In some embodiments, R′ is optionally substituted isoquinolinyl. In some embodiments, R′ is an optionally substituted 6,6-fused heteroaryl ring having two heteroatoms independently selected from nitrogen, oxygen, or sulfur. In some embodiments, R′ is optionally substituted quinazolinyl, phthalazinyl, quinoxalinyl or naphthyridinyl. In some embodiments, R′ is an optionally substituted 6,6-fused heteroaryl ring having three heteroatoms independently selected from nitrogen, oxygen, or sulfur. In some embodiments, R′ is optionally substituted pyridopyrimidinyl, pyridopyridazinyl, pyridopyrazinyl, or benzotriazinyl. In some embodiments, R′ is an optionally substituted 6,6-fused heteroaryl ring having four heteroatoms independently selected from nitrogen, oxygen, or sulfur. In some embodiments, R′ is optionally substituted pyridotriazinyl, pteridinyl, pyrazinopyrazinyl, pyrazinopyridazinyl, pyridazinopyridazinyl, pyrimidopyridazinyl or pyrimidopyrimidinyl. In some embodiments, R′ is an optionally substituted 6,6-fused heteroaryl ring having five heteroatoms independently selected from nitrogen, oxygen, or sulfur.

In some embodiments, R′ is optionally substituted heterobiaryl wherein each heteroaryl group is independently an optionally substituted group selected from a 5-6 membered monocyclic heteroaryl ring having 1-4 heteroatoms independently selected from nitrogen, oxygen, or sulfur, or an 8-10 membered bicyclic heteroaryl ring having 1-5 heteroatoms independently selected from nitrogen, oxygen, or sulfur. In some embodiments, R′ is optionally substituted heterobiaryl wherein each aryl group is an optionally substituted 8-10 membered bicyclic heteroaryl ring having 1-5 heteroatoms independently selected from nitrogen, oxygen, or sulfur.

In some embodiments, R′ is quinolinyl. In some embodiments, R′ is

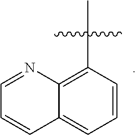 [SMILES: *c1cccc2cccnc12]

In some embodiments, Ar^{a} is optionally substituted

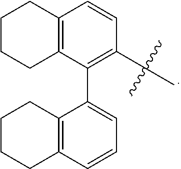 [SMILES: *c1ccc2c(c1-c1cccc3c1CCCC3)CCCC2]

In some embodiments, Ar^{a} is

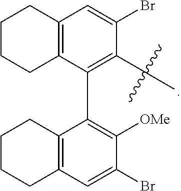 [SMILES: *c1c(Br)cc2c(c1-c1c3c(cc(Br)c1OC)CCCC3)CCCC2]

In some embodiments, Ar^{a} is

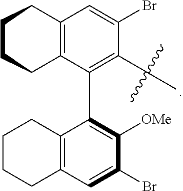

In some embodiments, R′ is symmetric. In some embodiments, R′ is asymmetric.

In some embodiments, —OAr^{a} is an optionally substituted group selected from:

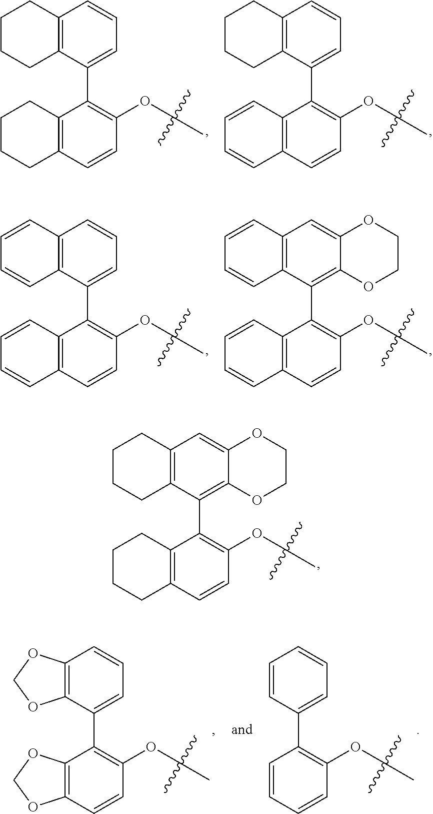

In some embodiments, —OAr^{a} is an optionally substituted group selected from:

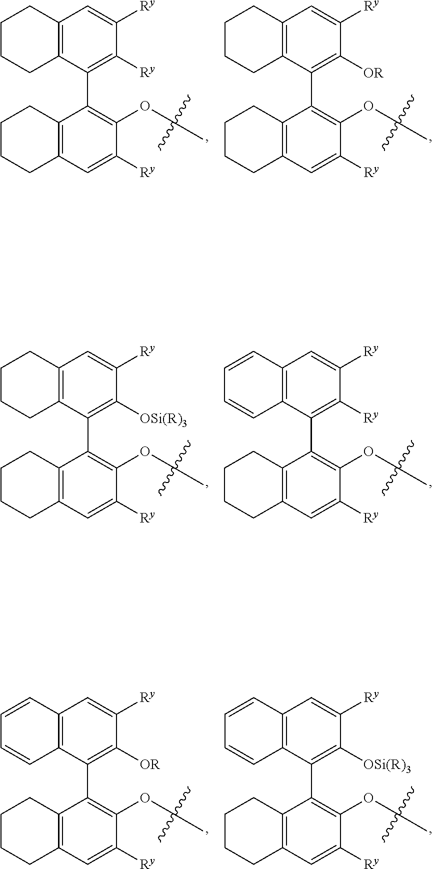

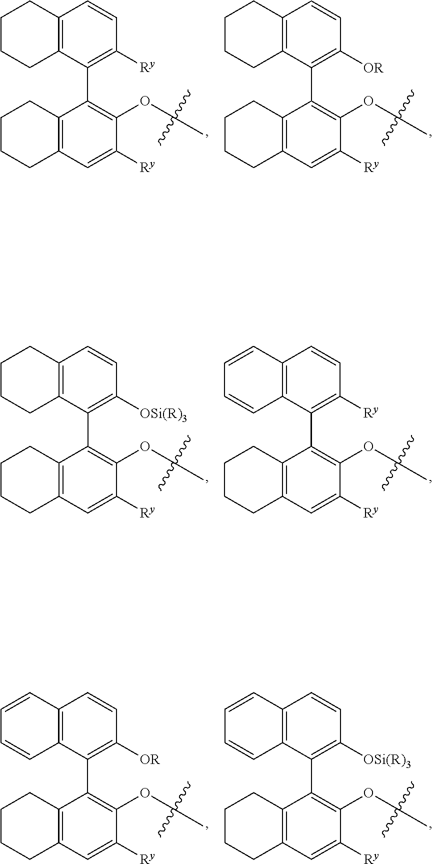

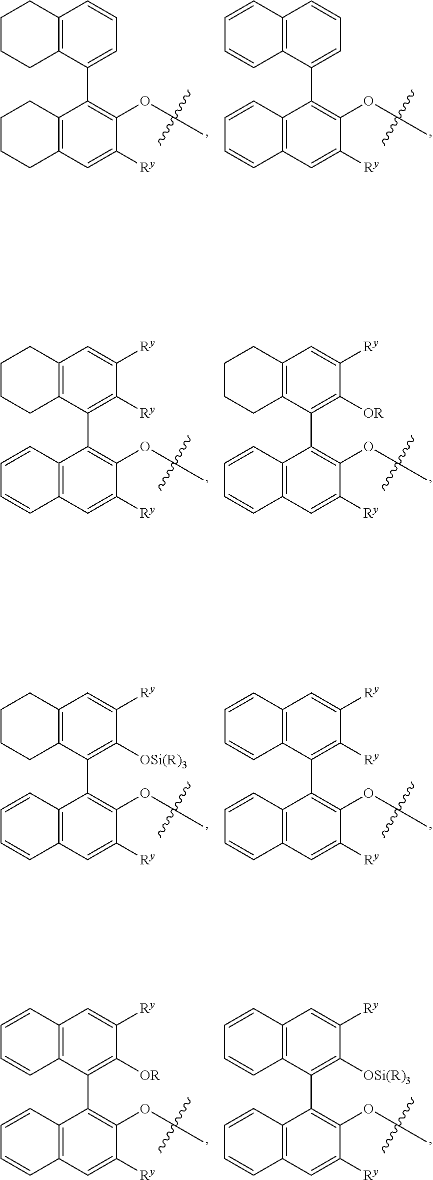

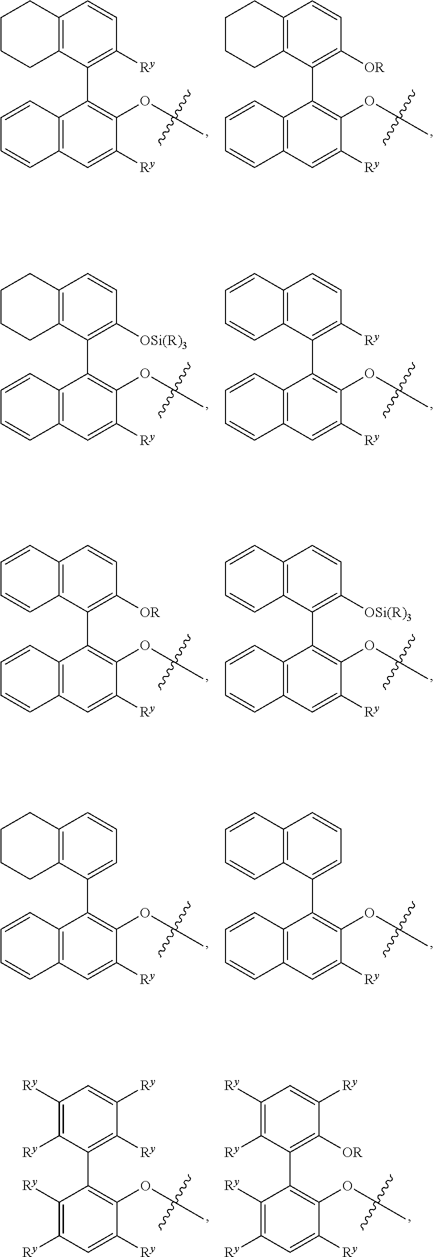

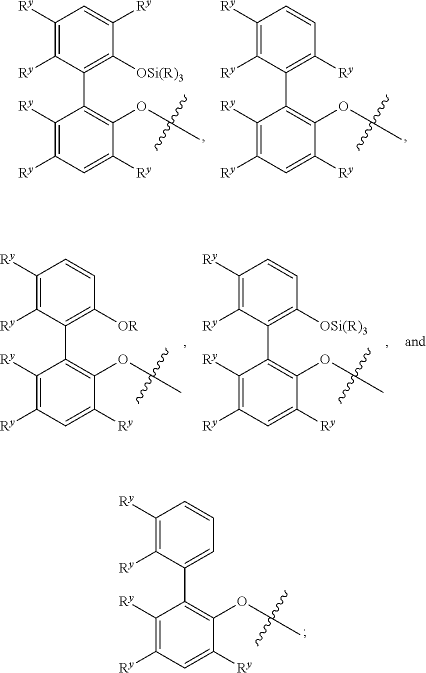

wherein each [symbol] represents the point of attachment to the metal, M, and each of R^{y} and R is independently as defined above and described herein. In some embodiments, one or more R^{y} is —F.

In some embodiments, —OAr^{a} is an optionally substituted group selected from:

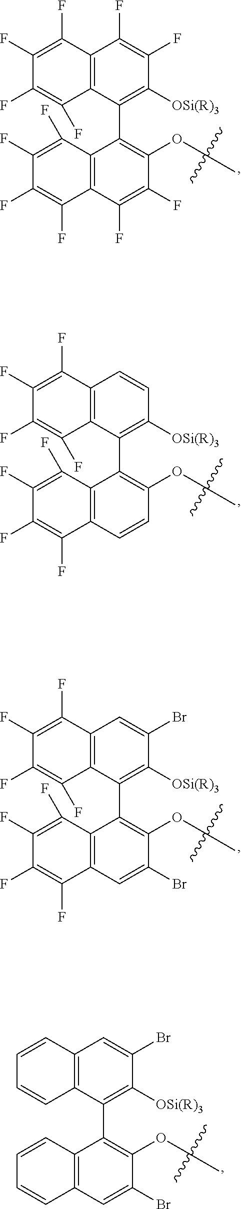

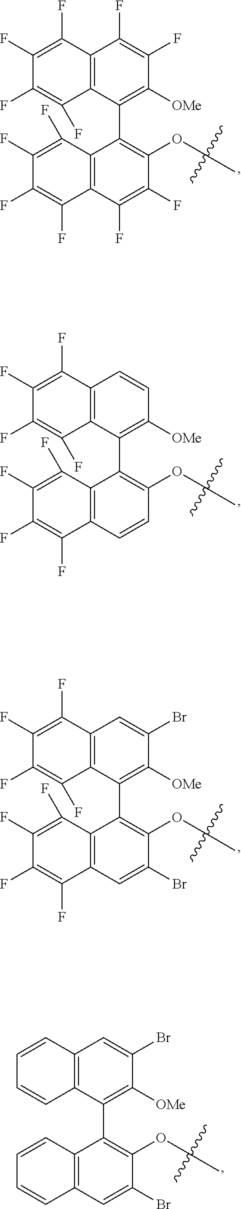 [SMILES: *Oc1c(F)c(F)c2c(F)c(F)c(F)c(F)c2c1-c1c(OC)c(F)c(F)c2c(F)c(F)c(F)c(F)c12 ; *Oc1ccc2c(F)c(F)c(F)c(F)c2c1-c1c(OC)ccc2c(F)c(F)c(F)c(F)c12 ; *Oc1c(Br)cc2c(F)c(F)c(F)c(F)c2c1-c1c(OC)c(Br)cc2c(F)c(F)c(F)c(F)c12 ; *Oc1c(Br)cc2ccccc2c1-c1c(OC)c(Br)cc2ccccc12]

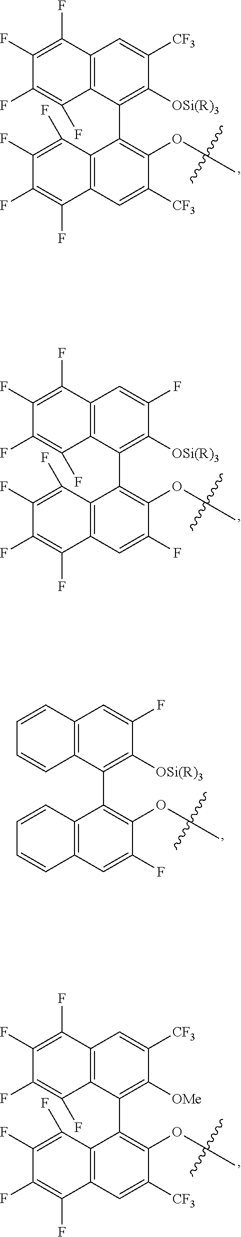

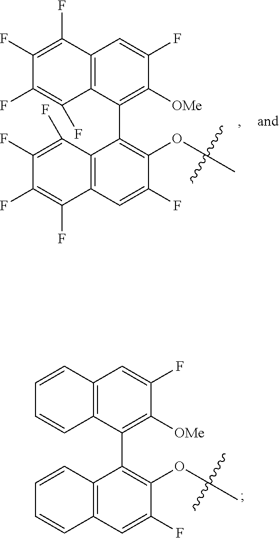 [SMILES: *Oc1c(F)cc2c(F)c(F)c(F)c(F)c2c1-c1c(OC)c(F)cc2c(F)c(F)c(F)c(F)c12 ; *Oc1c(F)cc2ccccc2c1-c1c(OC)c(F)cc2ccccc12]

wherein each [symbol] represents the point of attachment to the metal, M; and each of R^{y} and R is independently as defined above and described herein.

In some embodiments, —OR′ is

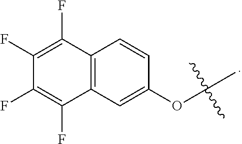 [SMILES: *Oc1ccc2c(F)c(F)c(F)c(F)c2c1]

As generally defined above, R″ is —Ar^{a}, or an optionally substituted group selected from phenyl, an 8-10 membered bicyclic aryl ring, a 5-6 membered monocyclic heteroaryl ring having 1-4 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R″ is Ar^{a}, wherein Ar^{a} is as defined above and described herein.

In some embodiments, R″ is an optionally substituted group selected from phenyl, an 8-10 membered bicyclic aryl ring, a 5-6 membered monocyclic heteroaryl ring having 1-4 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R″ is optionally substituted phenyl. In some embodiments, R″ is 2,6-terphenyl (Ter). In some embodiments, R″ is 2,6-bis(4′-methylphenyl)phenyl (Ter_{Me}). In some embodiments, R″ is 2,6-bis(4′-methoxyphenyl)phenyl (Ter_{OMe}). In some embodiments, R″ is 2,2′,6,6′-tetraisopropylterphenyl (TIPT). In some embodiments, R″ is 2,4,6-triisopropylphenyl (TRIP). In some embodiments, R″ is pentafluorophenyl (—C₆F₅). In some embodiments, R″ is 2,6-diisopropylphenyl. In some embodiments, R″ is 4-dimethylamino-2,6-diphenylphenyl. In some embodiments, R″ is 2,6-dimethoxylphenyl. In some embodiments, R″ is 2,6-diisopropylphenyl (Ar). In some embodiments, R″ is 2,6-dimethylphenyl (Ar′). In some embodiments, R″ is 2-(trifluoromethyl)phenyl (Ar^{CF3}). In some embodiments, R″ is 2-chlorophenyl (Ar^{Cl}). In some embodiments, R″ is 2-isopropylphenyl (Ar^{iPr}). In some embodiments, R″ is 2-biphenyl (Ar^{Ph}). In some embodiments, R″ is 3,5-dimethylphenyl (Ar^{m}). In some embodiments, R″ is 2-(2′,4′,6′-trimethylphenyl)phenyl (Ar^{m}). In some embodiments, R″ is 2-tert-butylphenyl (Ar^{tBu}). In some embodiments, R″ is 2-(2′,4′,6′-triisopropylphenyl)phenyl (Ar^{T}). In some embodiments, R″ is 2,6-bis(2′,4′,6′-triisopropylphenyl)phenyl (HIPT). In some embodiments, R″ is 2,6-bis(2′,4′,6′-trimethylphenyl)phenyl (HMT). In some embodiments, R″ is mesityl. In some embodiments, R″ is phenyl.

In some embodiments, R″ is optionally substituted phenyl wherein one or more substituents are halogen. In some embodiments, R″ is optionally substituted phenyl wherein one or more substituents are —F. In some embodiments, R″ is optionally substituted phenyl wherein one or more substituents are —Cl. In some embodiments, R″ is optionally substituted phenyl wherein one or more substituents are —Br. In some embodiments, R″ is optionally substituted phenyl wherein one or more substituents are —I.

In some embodiments, R″ is optionally substituted phenyl wherein one or more substituents are C₁₋₆ aliphatic. In some embodiments, R″ is optionally substituted phenyl wherein one or more substituents are C₁₋₆ phenyl. In some embodiments, R″ is 2,6-diisopropylphenyl (Ar). In some embodiments, R″ is 2,6-dimethylphenyl (Ar′). In some embodiments, R″ is 2-(trifluoromethyl)phenyl (Ar^{CF3}). In some embodiments, R″ is 2-chlorophenyl (Ar^{Cl}). In some embodiments, R″ is 2-isopropylphenyl (Ar^{iPr}). In some embodiments, R″ is 3,5-dimethylphenyl (Ar^{m}). In some embodiments, R″ is 2-tert-butylphenyl (Ar^{tBu}).

In some embodiments, R″ is an optionally substituted 8-10 membered bicyclic aryl ring. In some embodiments, R″ is an optionally substituted 5-6 membered monocyclic heteroaryl ring having 1-4 heteroatoms independently selected from nitrogen, oxygen, or sulfur. In some embodiments, R″ is an optionally substituted 8-10 membered bicyclic heteroaryl ring having 1-5 heteroatoms independently selected from nitrogen, oxygen, or sulfur.

Exemplary embodiments of R″ include but are not limited to those described for R′ wherein R′ is —Ar^{a}, or an optionally substituted group selected from phenyl, 8-10 membered bicyclic aryl ring, a 5-6 membered monocyclic heteroaryl ring having 1-4 heteroatoms independently selected from nitrogen, oxygen, or sulfur, or an 8-10 membered bicyclic heteroaryl ring having 1-5 heteroatoms independently selected from nitrogen, oxygen, or sulfur.

As generally defined above, Ar^{a} is:

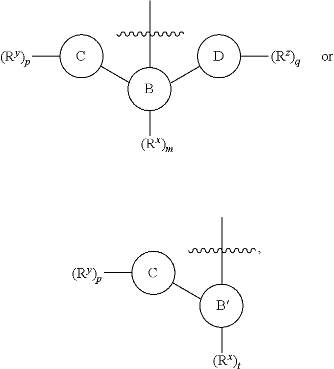

wherein each variable is independently as defined above and described herein.

As generally defined above, m is 0-3. In some embodiments, m is 0. In some embodiments, m is 1-3. In some embodiments, m is 1. In some embodiments, m is 2. In some embodiments, m is 3. In some embodiments, m is 0-2.

As generally defined above, Ring B is an optionally substituted group selected from phenyl or a 5-6 membered monocyclic heteroaryl ring having 1-4 heteroatoms independently selected from nitrogen, oxygen, or sulfur. In some embodiments, Ring B is of the following structure:

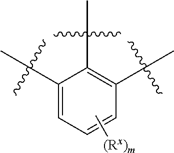

wherein R^{x} and m are as defined above and described herein. In some embodiments, Ring B is optionally substituted phenyl. In some embodiments, m=0. In some embodiments, Ring B is

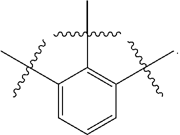 [SMILES: *c1cccc(*)c1*]

In some embodiments, Ring B is an optionally substituted 5-6 membered monocyclic heteroaryl ring having 1-4 heteroatoms independently selected from nitrogen, oxygen, or sulfur. In some embodiments, Ring B is an optionally substituted 5-membered monocyclic heteroaryl ring having 1-4 heteroatoms independently selected from nitrogen, oxygen, or sulfur, and m is 0-2. In some embodiments, Ring B is an optionally substituted 6-membered monocyclic heteroaryl ring having 1-4 heteroatoms independently selected from nitrogen, oxygen, or sulfur, and m is 0-3.

In some embodiments, Ring B is a 5-6 membered monocyclic heteroaryl ring having 1-4 heteroatoms independently selected from nitrogen, oxygen, or sulfur. In some embodiments, Ring B is a 5-6 membered monocyclic heteroaryl ring having 1-2 heteroatoms independently selected from nitrogen, oxygen, or sulfur.

In some embodiments, Ring B is a 5-membered monocyclic heteroaryl ring having 1-4 heteroatoms independently selected from nitrogen, oxygen, or sulfur. In some embodiments, Ring B is a 5-membered monocyclic heteroaryl ring having 1-2 heteroatoms independently selected from nitrogen, oxygen, or sulfur.

In some embodiments, Ring B is a 6-membered monocyclic heteroaryl ring having 1-4 heteroatoms independently selected from nitrogen, oxygen, or sulfur. In some embodiments, Ring B is a 6-membered monocyclic heteroaryl ring having 1-2 heteroatoms independently selected from nitrogen, oxygen, or sulfur.

Exemplary optionally substituted Ring B heteroaryl groups include thienylene, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, and the like.

As generally defined above, each of p and q is independently 0-5. In some embodiments, p is 0. In some embodiments, p is 1-5. In some embodiments, p is 1. In some embodiments, p is 2. In some embodiments, p is 3. In some embodiments, p is 4. In some embodiments, p is 5.

In some embodiments, q is 0. In some embodiments, q is 1-5. In some embodiments, q is 1. In some embodiments, q is 2. In some embodiments, q is 3. In some embodiments, q is 4. In some embodiments, q is 5.

In some embodiments, each of p and q is independently 1-5. In some embodiments, p is 1 and q is 1. In some embodiments, p is 2 and q is 2. In some embodiments, p is 2 and q is 2, and each of Ring C and Ring D independently has two substituents. In some embodiments, each of Ring C and Ring D has two substituents, and each substituent is at the o-position relative to the ring atom bonded to Ring B. In some embodiments, p is 3 and q is 3. In some embodiments, p is 4 and q is 4. In some embodiments, p is 5 and q is 5.

In some embodiments, p=q. In some embodiments, p is different from q.

As generally defined above, t is 0-4. In some embodiments, t is 0. In some embodiments, t is 1-4. In some embodiments, t is 1. In some embodiments, t is 2. In some embodiments, t is 3. In some embodiments, t is 4. In some embodiments, t is 0-2. In some embodiments, t is 0-3.

As generally defined above, each of Ring B′, Ring C and Ring D is independently an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-14 membered bicyclic or tricyclic heteroaryl ring having 1-5 heteroatoms independently selected from nitrogen, oxygen, or sulfur.

In some embodiments, Ring B′ is optionally substituted phenyl.

In some embodiments, Ring B′ is a group selected from:

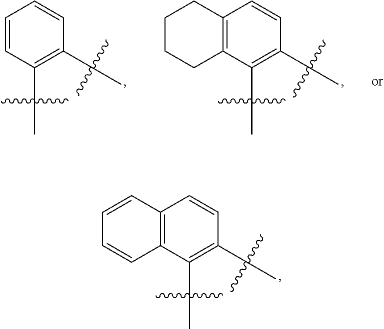 [SMILES: *c1ccccc1* ; *c1ccc2c(c1*)CCCC2 ; *c1ccc2ccccc2c1*]

wherein each [symbol] independently represents the point of attachment to Ring C or oxygen; wherein Ring B′ is optionally substituted with 0-4 R^{x}; and wherein each of Ring C′ and R^{x} is independently as defined above and described herein.

In some embodiments, Ring B′ is of the following formula:

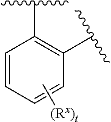

wherein each of R^{x} and t is independently as defined above and described herein

In some embodiments, Ring B′ is an optionally substituted 3-7 membered saturated carbocyclic ring. In some embodiments, Ring B′ is an optionally substituted 5-6 membered saturated carbocyclic ring. In some embodiments, Ring B′ is an optionally substituted 3-7 membered partially unsaturated carbocyclic ring. In some embodiments, Ring B′ is an optionally substituted 5-6 membered partially unsaturated carbocyclic ring.

In some embodiments, Ring B′ is an optionally substituted 8-10 membered bicyclic saturated carbocyclic ring. In some embodiments, Ring B′ is an optionally substituted 8-10 membered bicyclic partially unsaturated carbocyclic ring. In some embodiments, Ring B′ is an optionally substituted 8-10 membered bicyclic aryl ring.

In some embodiments, Ring B′ is an optionally substituted 5-6 membered monocyclic heteroaryl ring having 1-4 heteroatoms independently selected from nitrogen, oxygen, or sulfur. In some embodiments, Ring B′ is an optionally substituted 5-6 membered monocyclic heteroaryl ring having 1-2 heteroatoms independently selected from nitrogen, oxygen, or sulfur.

In some embodiments, Ring B′ is an optionally substituted 5 membered monocyclic heteroaryl ring having 1-4 heteroatoms independently selected from nitrogen, oxygen, or sulfur. In some embodiments, Ring B′ is an optionally substituted 5 membered monocyclic heteroaryl ring having 1-2 heteroatoms independently selected from nitrogen, oxygen, or sulfur.

In some embodiments, Ring B′ is an optionally substituted 6 membered monocyclic heteroaryl ring having 1-4 heteroatoms independently selected from nitrogen, oxygen, or sulfur. In some embodiments, Ring B′ is an optionally substituted 6 membered monocyclic heteroaryl ring having 1-2 heteroatoms independently selected from nitrogen, oxygen, or sulfur.

In some embodiments, Ring B′ is an optionally substituted 3-7 membered saturated heterocyclic ring having 1-3 heteroatoms independently selected from nitrogen, oxygen, or sulfur. In some embodiments, Ring B′ is an optionally substituted 5-6 membered saturated heterocyclic ring having 1-2 heteroatoms independently selected from nitrogen, oxygen, or sulfur.

In some embodiments, Ring B′ is an optionally substituted 3-7 membered partially unsaturated heterocyclic ring having 1-3 heteroatoms independently selected from nitrogen, oxygen, or sulfur. In some embodiments, Ring B′ is an optionally substituted 5-6 membered partially unsaturated heterocyclic ring having 1-2 heteroatoms independently selected from nitrogen, oxygen, or sulfur.

In some embodiments, Ring B′ is an optionally substituted 7-10 membered bicyclic saturated heterocyclic ring having 1-5 heteroatoms independently selected from nitrogen, oxygen, or sulfur. In some embodiments, Ring B′ is an optionally substituted 8-10 membered bicyclic saturated heterocyclic ring having 1-2 heteroatoms independently selected from nitrogen, oxygen, or sulfur.

In some embodiments, Ring B′ is an optionally substituted 7-10 membered bicyclic partially unsaturated heterocyclic ring having 1-5 heteroatoms independently selected from nitrogen, oxygen, or sulfur. In some embodiments, Ring B′ is an optionally substituted 8-10 membered bicyclic partially unsaturated heterocyclic ring having 1-2 heteroatoms independently selected from nitrogen, oxygen, or sulfur.

In some embodiments, Ring B′ is an optionally substituted 8-14 membered bicyclic or tricyclic heteroaryl ring having 1-5 heteroatoms independently selected from nitrogen, oxygen, or sulfur. In some embodiments, Ring B′ is an optionally substituted 8-10 membered bicyclic heteroaryl ring having 1-5 heteroatoms independently selected from nitrogen, oxygen, or sulfur. In some embodiments, Ring B′ is an optionally substituted 8 membered bicyclic heteroaryl ring having 1-3 heteroatoms independently selected from nitrogen, oxygen, or sulfur. In some embodiments, Ring B′ is an optionally substituted 9 membered bicyclic heteroaryl ring having 1-3 heteroatoms independently selected from nitrogen, oxygen, or sulfur. In some embodiments, Ring B′ is an optionally substituted 10 membered bicyclic heteroaryl ring having 1-3 heteroatoms independently selected from nitrogen, oxygen, or sulfur. In some embodiments, Ring B′ is a 10-14 membered tricyclic heteroaryl ring having 1-5 heteroatoms independently selected from nitrogen, oxygen, or sulfur.

In some embodiments, Ring C is of the following structure:

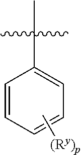

wherein R^{y} and p is independently as defined above and described herein.

In some embodiments, Ring D is of the following structure:

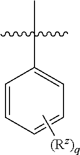

wherein R^{y} and p is independently as defined above and described herein.

In certain embodiments, Ring C is of the following formula:

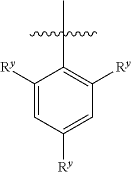

wherein R^{y} is as defined above and described herein.

In certain embodiments, Ring D is of the following formula:

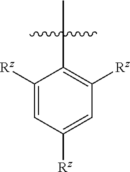

wherein R^{z} is as defined above and described herein.

In certain embodiments, Ring C or Ring D is of the following structure:

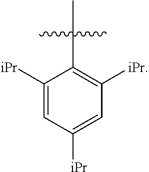 [SMILES: *c1c(C(C)C)cc(C(C)C)cc1C(C)C]

In some embodiments, Ring C is an optionally substituted a 3-7 membered saturated carbocyclic ring. In some embodiments, Ring C is an optionally substituted a 5-6 membered saturated carbocyclic ring. In some embodiments, Ring C is an optionally substituted a 3-7 membered partially unsaturated carbocyclic ring. In some embodiments, Ring C is an optionally substituted a 5-6 membered partially unsaturated carbocyclic ring.

In some embodiments, Ring D is an optionally substituted a 3-7 membered saturated carbocyclic ring. In some embodiments, Ring D is an optionally substituted a 5-6 membered saturated carbocyclic ring. In some embodiments, Ring D is an optionally substituted a 3-7 membered partially unsaturated carbocyclic ring. In some embodiments, Ring D is an optionally substituted a 5-6 membered partially unsaturated carbocyclic ring.

In some embodiments, Ring C is an optionally substituted 8-10 membered bicyclic saturated carbocyclic ring. In some embodiments, Ring C is an optionally substituted 8-10 membered bicyclic partially unsaturated carbocyclic ring. In some embodiments, Ring C is an optionally substituted 10 membered bicyclic aryl ring.

In some embodiments, Ring D is an optionally substituted 8-10 membered bicyclic saturated carbocyclic ring. In some embodiments, Ring D is an optionally substituted 8-10 membered bicyclic partially unsaturated carbocyclic ring. In some embodiments, Ring D is an optionally substituted 10 membered bicyclic aryl ring.

In some embodiments, Ring C is an optionally substituted 5-6 membered monocyclic heteroaryl ring having 1-4 heteroatoms independently selected from nitrogen, oxygen, or sulfur. In some embodiments, Ring C is an optionally substituted 5-6 membered monocyclic heteroaryl ring having 1-2 heteroatoms independently selected from nitrogen, oxygen, or sulfur. In some embodiments, Ring C is an optionally substituted 5 membered monocyclic heteroaryl ring having 1-4 heteroatoms independently selected from nitrogen, oxygen, or sulfur. In some embodiments, Ring C is an optionally substituted 5 membered monocyclic heteroaryl ring having 1-2 heteroatoms independently selected from nitrogen, oxygen, or sulfur. In some embodiments, Ring C is an optionally substituted 6 membered monocyclic heteroaryl ring having 1-4 heteroatoms independently selected from nitrogen, oxygen, or sulfur. In some embodiments, Ring C is an optionally substituted 6 membered monocyclic heteroaryl ring having 1-2 heteroatoms independently selected from nitrogen, oxygen, or sulfur.

In some embodiments, Ring D is an optionally substituted 5-6 membered monocyclic heteroaryl ring having 1-4 heteroatoms independently selected from nitrogen, oxygen, or sulfur. In some embodiments, Ring D is an optionally substituted 5-6 membered monocyclic heteroaryl ring having 1-2 heteroatoms independently selected from nitrogen, oxygen, or sulfur. In some embodiments, Ring D is an optionally substituted 5 membered monocyclic heteroaryl ring having 1-4 heteroatoms independently selected from nitrogen, oxygen, or sulfur. In some embodiments, Ring D is an optionally substituted 5 membered monocyclic heteroaryl ring having 1-2 heteroatoms independently selected from nitrogen, oxygen, or sulfur. In some embodiments, Ring D is an optionally substituted 6 membered monocyclic heteroaryl ring having 1-4 heteroatoms independently selected from nitrogen, oxygen, or sulfur. In some embodiments, Ring D is an optionally substituted 6 membered monocyclic heteroaryl ring having 1-2 heteroatoms independently selected from nitrogen, oxygen, or sulfur.

In some embodiments, Ring C is an optionally substituted 3-7 membered saturated heterocyclic ring having 1-3 heteroatoms independently selected from nitrogen, oxygen, or sulfur. In some embodiments, Ring C is an optionally substituted 5-6 membered saturated heterocyclic ring having 1-2 heteroatoms independently selected from nitrogen, oxygen, or sulfur. In some embodiments, Ring C is an optionally substituted 3-7 membered partially unsaturated heterocyclic ring having 1-3 heteroatoms independently selected from nitrogen, oxygen, or sulfur. In some embodiments, Ring C is an optionally substituted 5-6 membered partially unsaturated heterocyclic ring having 1-2 heteroatoms independently selected from nitrogen, oxygen, or sulfur.

In some embodiments, Ring D is an optionally substituted 3-7 membered saturated heterocyclic ring having 1-3 heteroatoms independently selected from nitrogen, oxygen, or sulfur. In some embodiments, Ring D is an optionally substituted 5-6 membered saturated heterocyclic ring having 1-2 heteroatoms independently selected from nitrogen, oxygen, or sulfur. In some embodiments, Ring D is an optionally substituted 3-7 membered partially unsaturated heterocyclic ring having 1-3 heteroatoms independently selected from nitrogen, oxygen, or sulfur. In some embodiments, Ring D is an optionally substituted 5-6 membered partially unsaturated heterocyclic ring having 1-2 heteroatoms independently selected from nitrogen, oxygen, or sulfur.

In some embodiments, Ring C is an optionally substituted 7-10 membered bicyclic saturated heterocyclic ring having 1-5 heteroatoms independently selected from nitrogen, oxygen, or sulfur. In some embodiments, Ring C is an optionally substituted 8-10 membered bicyclic saturated heterocyclic ring having 1-2 heteroatoms independently selected from nitrogen, oxygen, or sulfur. In some embodiments, Ring C is an optionally substituted 7-10 membered bicyclic partially unsaturated heterocyclic ring having 1-5 heteroatoms independently selected from nitrogen, oxygen, or sulfur. In some embodiments, Ring C is an optionally substituted 8-10 membered bicyclic partially unsaturated heterocyclic ring having 1-2 heteroatoms independently selected from nitrogen, oxygen, or sulfur.

In some embodiments, Ring D is an optionally substituted 7-10 membered bicyclic saturated heterocyclic ring having 1-5 heteroatoms independently selected from nitrogen, oxygen, or sulfur. In some embodiments, Ring D is an optionally substituted 8-10 membered bicyclic saturated heterocyclic ring having 1-2 heteroatoms independently selected from nitrogen, oxygen, or sulfur. In some embodiments, Ring D is an optionally substituted 7-10 membered bicyclic partially unsaturated heterocyclic ring having 1-5 heteroatoms independently selected from nitrogen, oxygen, or sulfur. In some embodiments, Ring D is an optionally substituted 8-10 membered bicyclic partially unsaturated heterocyclic ring having 1-2 heteroatoms independently selected from nitrogen, oxygen, or sulfur.

In some embodiments, Ring C is an optionally substituted 8-14 membered bicyclic or tricyclic heteroaryl ring having 1-5 heteroatoms independently selected from nitrogen, oxygen, or sulfur. In some embodiments, Ring C is an optionally substituted 8-10 membered bicyclic heteroaryl ring having 1-5 heteroatoms independently selected from nitrogen, oxygen, or sulfur. In some embodiments, Ring C is an optionally substituted 8 membered bicyclic heteroaryl ring having 1-3 heteroatoms independently selected from nitrogen, oxygen, or sulfur. In some embodiments, Ring C is an optionally substituted 9 membered bicyclic heteroaryl ring having 1-3 heteroatoms independently selected from nitrogen, oxygen, or sulfur. In some embodiments, Ring C is an optionally substituted 10 membered bicyclic heteroaryl ring having 1-3 heteroatoms independently selected from nitrogen, oxygen, or sulfur. In some embodiments, Ring C is an optionally substituted 10-14 membered tricyclic heteroaryl ring having 1-5 heteroatoms independently selected from nitrogen, oxygen, or sulfur.

In some embodiments, Ring D is an optionally substituted 8-14 membered bicyclic or tricyclic heteroaryl ring having 1-5 heteroatoms independently selected from nitrogen, oxygen, or sulfur. In some embodiments, Ring D is an optionally substituted 8-10 membered bicyclic heteroaryl ring having 1-5 heteroatoms independently selected from nitrogen, oxygen, or sulfur. In some embodiments, Ring D is an optionally substituted 8 membered bicyclic heteroaryl ring having 1-3 heteroatoms independently selected from nitrogen, oxygen, or sulfur. In some embodiments, Ring D is an optionally substituted 9 membered bicyclic heteroaryl ring having 1-3 heteroatoms independently selected from nitrogen, oxygen, or sulfur. In some embodiments, Ring D is an optionally substituted 10 membered bicyclic heteroaryl ring having 1-3 heteroatoms independently selected from nitrogen, oxygen, or sulfur. In some embodiments, Ring D is an optionally substituted 10-14 membered tricyclic heteroaryl ring having 1-5 heteroatoms independently selected from nitrogen, oxygen, or sulfur.

Exemplary embodiments for Ring C include but are not limited to those described for R′ wherein R′ is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur. Exemplary embodiments for Ring C include but are not limited to those described for R′ wherein R′ is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

As generally defined above, each of R^{x}, R^{y}, and R^{z} is independently halogen, R, —OR, —SR, —S(O)R, —S(O)₂R, —OSi(R)₃, —N(R)₂, —NRC(O)R, —NRC(O)OR, —NRC(O)N(R)₂, —NRSO₂R, —NRSO₂N(R)₂, or —NROR, wherein each R is independently as defined above and described herein.

In some embodiments, R^{x} is halogen. In some embodiments, R^{x} is —F. In some embodiments, R^{x} is —Cl. In some embodiments, R^{x} is —Br. In some embodiments, R^{x} is —I.

In some embodiments, R^{x} is R, —OR, —SR, —S(O)R, —S(O)₂R, —OSi(R)₃, —N(R)₂, —NRC(O)R, —NRC(O)OR, —NRC(O)N(R)₂, —NRSO₂R, —NRSO₂N(R)₂, or —NROR, wherein each R is independently as defined above and described herein.

In certain embodiments, R^{x} is R, wherein R is as defined above and described herein. In some embodiments, R^{x} is optionally substituted C₁₋₆ aliphatic. In some embodiments, R^{x} is optionally substituted C₁₋₆ alkyl. In some embodiments, R^{x} is optionally substituted C₁₋₆ haloalkyl. In some embodiments, R^{x} is optionally substituted C₁₋₆ haloalkyl, wherein one substituent is —F. In some embodiments, R^{x} is optionally substituted C₁₋₆ haloalkyl, wherein two or more substituents are —F. In certain embodiments, R^{x} is selected from methyl, ethyl, propyl, or butyl. In certain embodiments, R^{x} is isopropyl. In certain embodiments, R^{x} is —CF₃.

In some embodiments, R^{x} is hydrogen. In some embodiments, R^{x} is an optionally substituted group selected from C₁₋₆ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R^{x} is optionally substituted phenyl. In some embodiments, R^{x} is substituted phenyl. In some embodiments, R^{x} is phenyl.

In some embodiments, R^{x} is —OR, wherein each R is independently as defined above and described herein. In some embodiments, R^{x} is —OMe.

In some embodiments, R^{x} is selected from —SR, —S(O)R, —S(O)₂R, wherein each R is independently as defined above and described herein.

In some embodiments, R^{x} is —OSi(R)₃, wherein each R is independently as defined above and described herein.

In some embodiments, R^{x} is —N(R²), wherein each R is independently as defined above and described herein. In some embodiments, R^{x} is —N(Me)₂.

In some embodiments, R^{x} is —NRC(O)R, —NRC(O)OR, —NRC(O)N(R)₂, —NRSO₂R, —NRSO₂N(R)₂, or —NROR, wherein each R is independently as defined above and described herein.

In some embodiments, R^{y} is halogen. In some embodiments, R^{y} is —F. In some embodiments, R^{y} is —Cl. In some embodiments, R^{y} is —Br. In some embodiments, R^{y} is —I.

In some embodiments, R^{y} is R, —OR, —SR, —S(O)R, —S(O)₂R, —OSi(R)₃, —N(R)₂, —NRC(O)R, —NRC(O)OR, —NRC(O)N(R)₂, —NRSO₂R, —NRSO₂N(R)₂, or —NROR, wherein each R is independently as defined above and described herein.

In certain embodiments, R^{y} is R, wherein R is as defined above and described herein. In some embodiments, R^{y} is optionally substituted C₁₋₆ aliphatic. In some embodiments, R^{y} is optionally substituted C₁₋₆ alkyl. In some embodiments, R^{y} is optionally substituted C₁₋₆ haloalkyl. In some embodiments, R^{y} is optionally substituted C₁₋₆ haloalkyl, wherein one substituent is —F. In some embodiments, R^{y} is optionally substituted C₁₋₆ haloalkyl, wherein two or more substituents are —F. In certain embodiments, R^{y} is selected from methyl, ethyl, propyl, or butyl. In certain embodiments, R^{y} is isopropyl. In certain embodiments, R^{y} is —CF₃.

In some embodiments, R^{y} is hydrogen. In some embodiments, R^{y} is an optionally substituted group selected from C₁₋₆ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R^{y} is optionally substituted phenyl. In some embodiments, R^{y} is substituted phenyl. In some embodiments, R^{y} is phenyl.

In some embodiments, R^{y} is —OR, wherein each R is independently as defined above and described herein. In some embodiments, R^{y} is —OMe.

In some embodiments, R^{y} is selected from —SR, —S(O)R, —S(O)₂R, wherein each R is independently as defined above and described herein.

In some embodiments, R^{y} is —OSi(R)₃, wherein each R is independently as defined above and described herein.

In some embodiments, R^{y} is —N(R²), wherein each R is independently as defined above and described herein. In some embodiments, R^{y} is —N(Me)₂.

In some embodiments, R^{y} is —NRC(O)R, —NRC(O)OR, —NRC(O)N(R)₂, —NRSO₂R, —NRSO₂N(R)₂, or —NROR, wherein each R is independently as defined above and described herein.

In some embodiments, R^{z} is halogen. In some embodiments, R^{z} is —F. In some embodiments, R^{z} is —Cl. In some embodiments, R^{z} is —Br. In some embodiments, R^{z} is —I.

In some embodiments, R^{z} is R, —OR, —SR, —S(O)R, —S(O)₂R, —OSi(R)₃, —N(R)₂, —NRC(O)R, —NRC(O)OR, —NRC(O)N(R)₂, —NRSO₂R, —NRSO₂N(R)₂, or —NROR, wherein each R is independently as defined above and described herein.

In certain embodiments, R^{z} is R, wherein R is as defined above and described herein. In some embodiments, R^{z} is optionally substituted C₁₋₆ aliphatic. In some embodiments, R^{z} is optionally substituted C₁₋₆ alkyl. In some embodiments, R^{z} is optionally substituted C₁₋₆ haloalkyl. In some embodiments, R^{z} is optionally substituted C₁₋₆ haloalkyl, wherein one substituent is —F. In some embodiments, R^{z} is optionally substituted C₁₋₆ haloalkyl, wherein two or more substituents are —F. In certain embodiments, R^{z} is selected from methyl, ethyl, propyl, or butyl. In certain embodiments, R^{z} is isopropyl. In certain embodiments, R^{z} is —CF₃.

In some embodiments, R^{z} is hydrogen. In some embodiments, R^{z} is an optionally substituted group selected from C₁₋₆ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur.

In some embodiments, R^{z} is optionally substituted phenyl. In some embodiments, R^{z} is substituted phenyl. In some embodiments, R^{z} is phenyl.

In some embodiments, R^{z} is —OR, wherein each R is independently as defined above and described herein. In some embodiments, R^{z} is —OMe.

In some embodiments, R^{z} is selected from —SR, —S(O)R, —S(O)₂R, wherein each R is independently as defined above and described herein.

In some embodiments, R^{z} is —OSi(R)₃, wherein each R is independently as defined above and described herein.

In some embodiments, R^{z} is —N(R²), wherein each R is independently as defined above and described herein. In some embodiments, R^{z} is —N(Me)₂.

In some embodiments, R^{z} is —NRC(O)R, —NRC(O)OR, —NRC(O)N(R)₂, —NRSO₂R, —NRSO₂N(R)₂, or —NROR, wherein each R is independently as defined above and described herein.

In some embodiments, R^{x} is an electron-withdrawing group. In some embodiments, R^{y} is an electron-withdrawing group. In some embodiments, R^{z} is an electron-withdrawing group.

In some embodiments, Ar^{a} is of the formula:

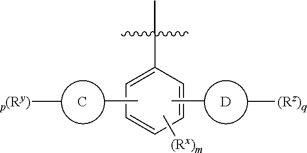

wherein each of R^{x}, m, Ring C, R^{y}, p, Ring D, R^{z}, and q is as defined above and described herein.

In some embodiments, Ar^{a} is of the formula:

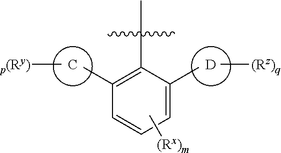

wherein each of R^{x}, m, Ring C, R^{y}, p, Ring D, R^{z}, and q are as defined above and described herein.

In some embodiments, Ar^{a} is of the formula:

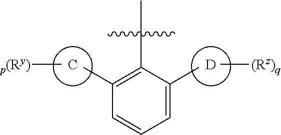

wherein each of Ring C, R^{y}, p, Ring D, R^{z}, and q are as defined above and described herein.

In some embodiments, Ar^{a} is of the formula:

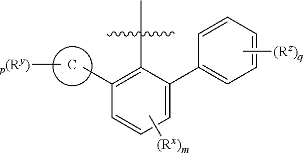

wherein each of R^{x}, m, Ring C, R^{y}, p, R^{z}, and q are as defined above and described herein.

In some embodiments, Ar^{a} is of the formula:

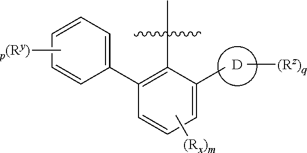

wherein each of R^{x}, m, Ring C, R^{y}, p, Ring D, R^{z}, and q are as defined above and described herein.

In some embodiments, Ar^{a} is of the formula:\

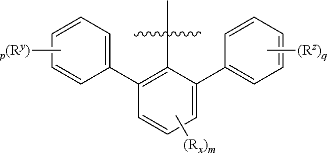

wherein each of R^{x}, m, R^{y}, p, R^{z}, and q are as defined above and described herein.

In some embodiments, Ar^{a} is of the formula:

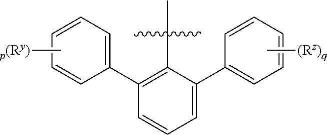

wherein each of R^{y}, p, R^{z}, and q are as defined above and described herein.

In some embodiments, Ar^{a} is of the formula:

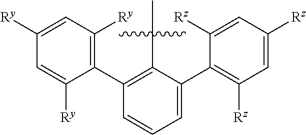

wherein each of R^{y} and R^{z} are as defined above and described herein. In certain embodiments wherein —Ar is as depicted above, each R^{y} and each R^{z} is independently selected from optionally substituted C₁₋₂₀ aliphatic. In certain embodiments wherein —Ar is as depicted above, each R^{y} and each R^{z} is independently selected from optionally substituted C₁₋₁₀ aliphatic. In certain embodiments wherein —Ar is as depicted above, each R^{y} and each R^{z} is independently selected from optionally substituted alkyl. Exemplary R^{y} and R^{z} groups include methyl, ethyl, propyl, and butyl.

In some embodiments, Ar^{a} is an optionally substituted group selected from:

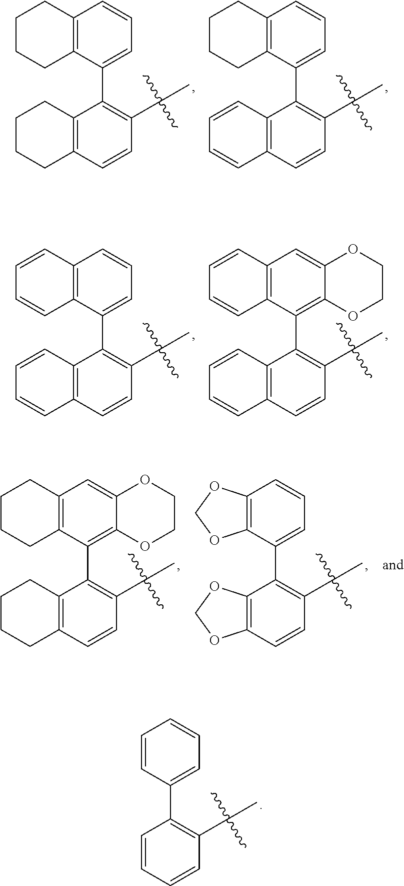

In some embodiments, Ara is an optionally substituted group selected from:

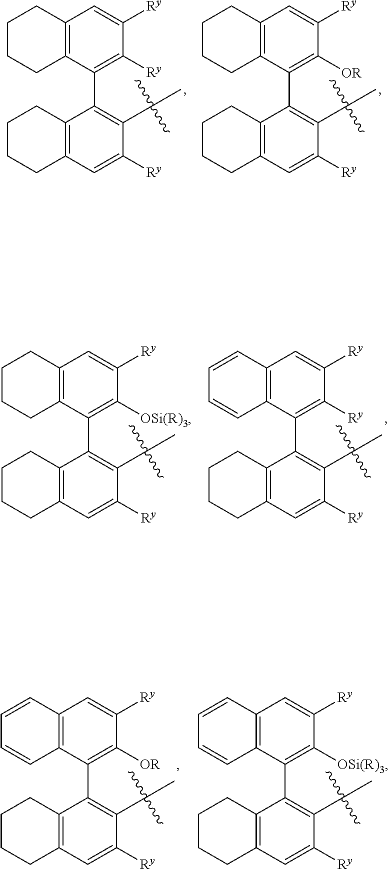

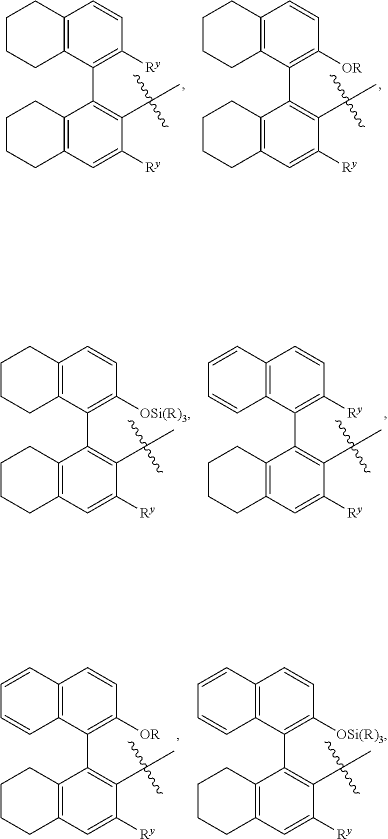

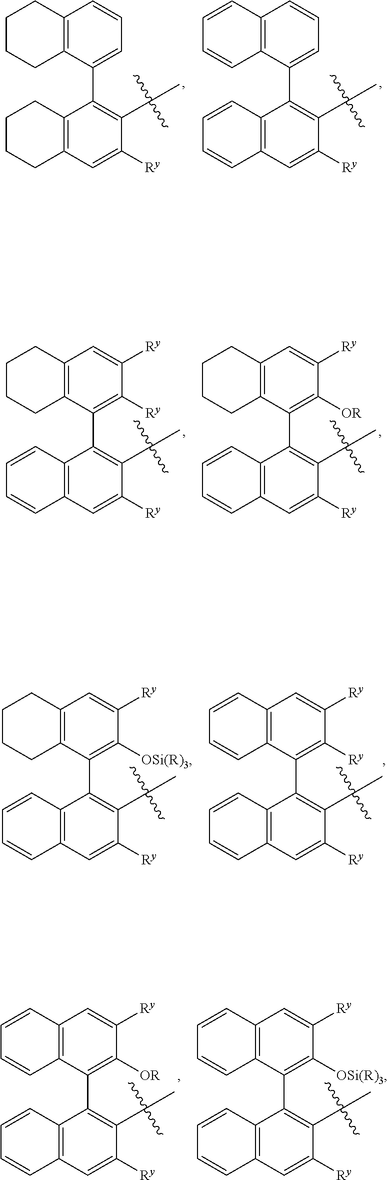

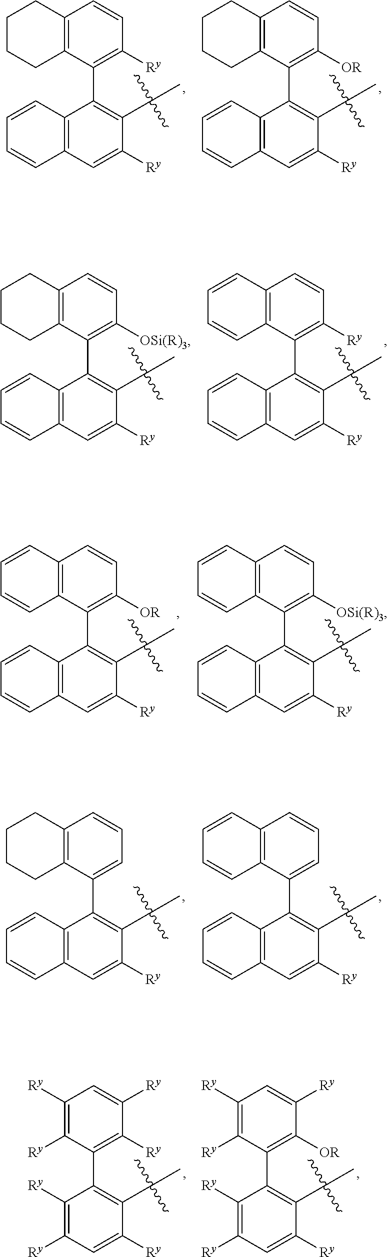

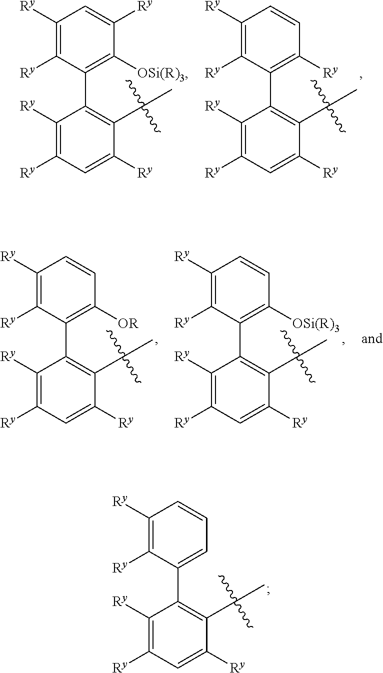

wherein each of R^{y} and R is independently as defined above and described herein. In some embodiments, one or more R^{y} is —F.

In some embodiments, Ara is an optionally substituted group selected from:

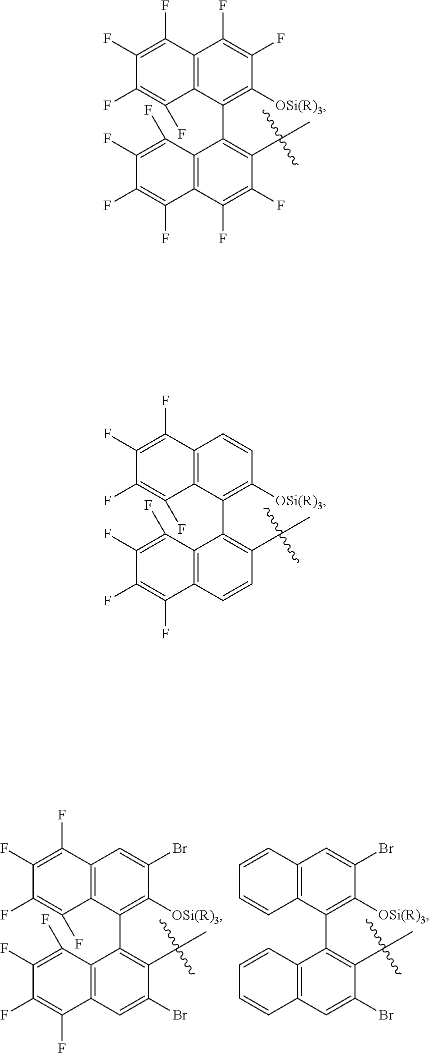

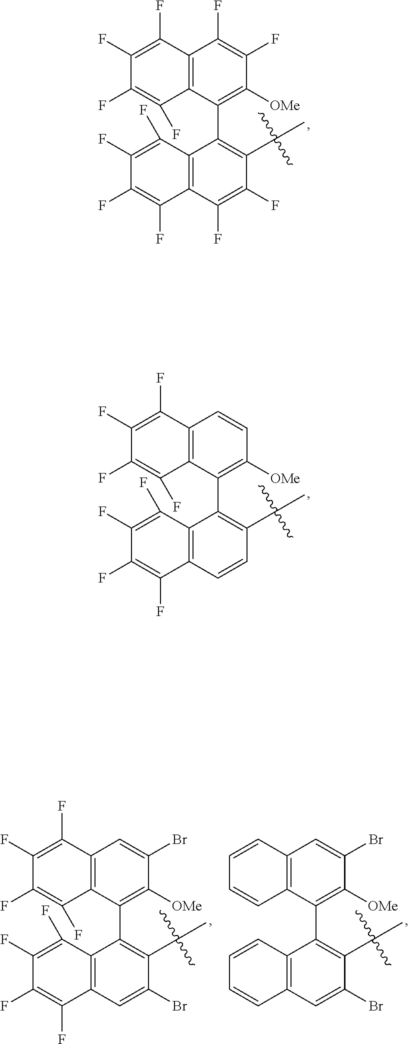 [SMILES: *c1c(F)c(F)c2c(F)c(F)c(F)c(F)c2c1-c1c(OC)c(F)c(F)c2c(F)c(F)c(F)c(F)c12 ; *c1ccc2c(F)c(F)c(F)c(F)c2c1-c1c(OC)ccc2c(F)c(F)c(F)c(F)c12 ; *c1c(Br)cc2c(F)c(F)c(F)c(F)c2c1-c1c(OC)c(Br)cc2c(F)c(F)c(F)c(F)c12 ; *c1c(Br)cc2ccccc2c1-c1c(OC)c(Br)cc2ccccc12]

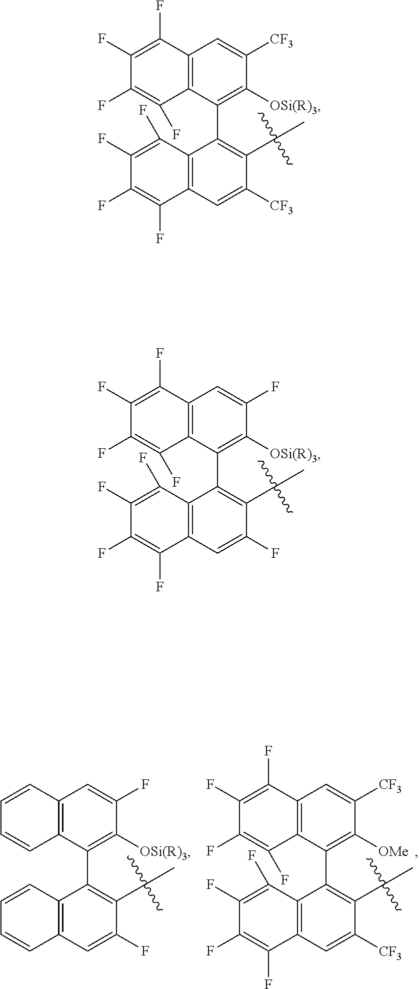

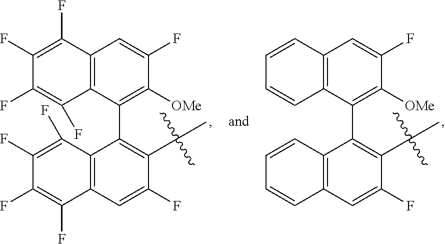 [SMILES: *c1c(F)cc2c(F)c(F)c(F)c(F)c2c1-c1c(OC)c(F)cc2c(F)c(F)c(F)c(F)c12 ; *c1c(F)cc2ccccc2c1-c1c(OC)c(F)cc2ccccc12]

wherein each of R^{y} and R is independently as defined above and described herein.

In some embodiments, R′ is

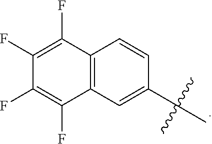 [SMILES: *c1ccc2c(F)c(F)c(F)c(F)c2c1]

In some embodiments, Ara is

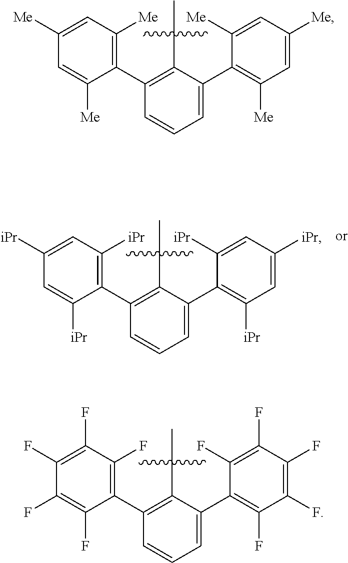 [SMILES: *c1c(-c2c(C)cc(C)cc2C)cccc1-c1c(C)cc(C)cc1C ; *c1c(-c2c(C(C)C)cc(C(C)C)cc2C(C)C)cccc1-c1c(C(C)C)cc(C(C)C)cc1C(C)C ; "or" ; *c1c(-c2c(F)c(F)c(F)c(F)c2F)cccc1-c1c(F)c(F)c(F)c(F)c1F]

As generally defined above, each R is independently hydrogen or an optionally substituted group selected from C₁₋₂₀ aliphatic, C₁₋₂₀ heteroaliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur; or:
two R groups on the same atom are optionally taken together with the atom to which they are attached to form an optionally substituted 3-10 membered, monocyclic or bicyclic, saturated, partially unsaturated, or aryl ring having, in addition to the atom to which they are attached, 0-4 heteroatoms independently selected from nitrogen, oxygen, or sulfur.

In some embodiments, each R is independently hydrogen or an optionally substituted group selected from C₁₋₂₀ aliphatic, C₁₋₂₀ heteroaliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur. In some embodiments, two R groups on the same atom are optionally taken together with the atom to which they are attached to form an optionally substituted 3-10 membered, monocyclic or bicyclic, saturated, partially unsaturated, or aryl ring having, in addition to the atom to which they are attached, 0-4 heteroatoms independently selected from nitrogen, oxygen, or sulfur.

In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C₁₋₆ aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl.

In some embodiments, R is optionally substituted C₁₋₂₀ heteroaliphatic. In some embodiments, R is optionally substituted C₁₋₂₀ heteroaliphatic having 1-6 heteroatoms independently selected from nitrogen, sulfur, phosphorus or selenium. In some embodiments, R is optionally substituted C₁₋₂₀ heteroaliphatic having 1-6 heteroatoms independently selected from nitrogen, sulfur, phosphorus or selenium, optionally including one or more oxidized forms of nitrogen, sulfur, phosphorus or selenium. In some embodiments, R is optionally substituted C₁₋₂₀ heteroaliphatic comprising 1-6 groups independently selected from

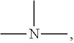

### —N═, ≡N, —S—, —S(O)—, —S(O)₂—, —O—, ═O,

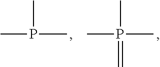

### —Se—, and —Se(O)—.

In some embodiments, R is optionally substituted phenyl. In some embodiments, R is optionally substituted phenyl wherein one or more substituents are halogen. In some embodiments, R is optionally substituted phenyl wherein one or more substituents are —F. In some embodiments, R is optionally substituted phenyl wherein one or more substituents are —Cl. In some embodiments, R is optionally substituted phenyl wherein one or more substituents are —Br. In some embodiments, R is optionally substituted phenyl wherein one or more substituents are —I. In some embodiments, R is phenyl.

In some embodiments, R is an optionally substituted 3-7 membered saturated or partially unsaturated carbocyclic ring. In some embodiments, R is an optionally substituted 3-membered saturated or partially unsaturated carbocyclic ring. In some embodiments, R is an optionally substituted 4-membered saturated or partially unsaturated carbocyclic ring. In some embodiments, R is an optionally substituted 5-membered saturated or partially unsaturated carbocyclic ring. In some embodiments, R is an optionally substituted 6-membered saturated or partially unsaturated carbocyclic ring. In some embodiments, R is an optionally substituted 7-membered saturated or partially unsaturated carbocyclic ring. In some embodiments, R is optionally substituted cycloheptyl. In some embodiments, R is osub cycloheptyl. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, R is an optionally substituted 8-10 membered bicyclic saturated, partially unsaturated or aryl ring. In some embodiments, R is an optionally substituted 8-10 membered bicyclic saturated ring. In some embodiments, R is an optionally substituted 8-10 membered bicyclic partially unsaturated ring. In some embodiments, R is an optionally substituted 8-10 membered bicyclic aryl ring. In some embodiments, R is optionally substituted naphthyl.

In some embodiments, R is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur. In some embodiments, R is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is optionally substituted phenyl. In some embodiments, R is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is an optionally substituted 5-6 membered monocyclic heteroaryl ring having 1-4 heteroatoms independently selected from nitrogen, oxygen, or sulfur. In some embodiments, R is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is an optionally substituted 8-10 membered bicyclic aryl ring. In some embodiments, R is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is optionally substituted naphthyl. In some embodiments, R is optionally substituted biaryl wherein each aryl group is independently an optionally substituted group selected from phenyl, 5-6 membered monocyclic heteroaryl ring having 1-4 heteroatoms independently selected from nitrogen, oxygen, or sulfur, an 8-10 membered bicyclic aryl ring, or an 8-10 membered bicyclic heteroaryl ring having 1-5 heteroatoms independently selected from nitrogen, oxygen, or sulfur, and wherein at least one aryl group is an optionally substituted 8-10 membered bicyclic heteroaryl ring having 1-5 heteroatoms independently selected from nitrogen, oxygen, or sulfur. In some embodiments, R is optionally substituted biaryl wherein each aryl group is independently optionally substituted phenyl. In some embodiments, R is optionally substituted biaryl wherein each aryl group is independently optionally substituted phenyl, or an optionally substituted 5-6 membered monocyclic heteroaryl ring having 1-4 heteroatoms independently selected from nitrogen, oxygen. In some embodiments, R is optionally substituted biaryl wherein each aryl group is independently an optionally substituted 8-10 membered bicyclic aryl ring. In some embodiments, R is optionally substituted biaryl wherein one aryl group is optionally substituted naphthyl, and the other aryl group is independently an optionally substituted 8-10 membered bicyclic aryl ring. In some embodiments, R is optionally substituted biaryl wherein each aryl group is optionally substituted naphthyl. In some embodiments, R is optionally substituted biaryl wherein one aryl group is optionally substituted naphthyl, and the other aryl group is an optionally substituted 8-10 membered bicyclic heteroaryl ring having 1-5 heteroatoms independently selected from nitrogen, oxygen, or sulfur.

In some embodiments, R is an optionally substituted 5-6 membered monocyclic heteroaryl ring having 1-4 heteroatoms independently selected from nitrogen, oxygen, or sulfur. In some embodiments, R is a substituted 5-6 membered monocyclic heteroaryl ring having 1-4 heteroatoms independently selected from nitrogen, oxygen, or sulfur. In some embodiments, R is an unsubstituted 5-6 membered monocyclic heteroaryl ring having 1-4 heteroatoms independently selected from nitrogen, oxygen, or sulfur.

In some embodiments, R is an optionally substituted 5-membered monocyclic heteroaryl ring having 1-4 heteroatoms independently selected from nitrogen, oxygen or sulfur. In some embodiments, R is an optionally substituted 6-membered monocyclic heteroaryl ring having 1-4 heteroatoms independently selected from nitrogen, oxygen, or sulfur.

In some embodiments, R is an optionally substituted 5-membered monocyclic heteroaryl ring having one heteroatom selected from nitrogen, oxygen, or sulfur. In some embodiments, R is selected from optionally substituted pyrrolyl, furanyl, or thienyl.

In some embodiments, R is an optionally substituted 5-membered heteroaryl ring having two heteroatoms independently selected from nitrogen, oxygen, or sulfur. In certain embodiments, R is an optionally substituted 5-membered heteroaryl ring having one nitrogen atom, and an additional heteroatom selected from sulfur or oxygen. Exemplary R groups include but are not limited to optionally substituted pyrazolyl, imidazolyl, thiazolyl, isothiazolyl, oxazolyl or isoxazolyl.

In some embodiments, R is an optionally substituted 5-membered heteroaryl ring having three heteroatoms independently selected from nitrogen, oxygen, or sulfur. Exemplary R groups include but are not limited to optionally substituted triazolyl, oxadiazolyl or thiadiazolyl.

In some embodiments, R is an optionally substituted 5-membered heteroaryl ring having four heteroatoms independently selected from nitrogen, oxygen, or sulfur. Exemplary R groups include but are not limited to optionally substituted tetrazolyl, oxatriazolyl and thiatriazolyl.

In some embodiments, R is an optionally substituted 6-membered heteroaryl ring having 1-4 nitrogen atoms. In some embodiments, R is a 6-membered heteroaryl ring having 1-3 nitrogen atoms. In other embodiments, R is an optionally substituted 6-membered heteroaryl ring having 1-2 nitrogen atoms. In some embodiments, R is an optionally substituted 6-membered heteroaryl ring having four nitrogen atoms. In some embodiments, R is an optionally substituted 6-membered heteroaryl ring having three nitrogen atoms. In some embodiments, R is an optionally substituted 6-membered heteroaryl ring having two nitrogen atoms. In certain embodiments, R is an optionally substituted 6-membered heteroaryl ring having one nitrogen atom. Exemplary R groups include but are not limited to optionally substituted pyridinyl, pyrimidinyl, pyrazinyl, pyridazinyl, triazinyl, or tetrazinyl.

In some embodiments, R is an optionally substituted 3-7 membered saturated or partially unsaturated heterocyclic ring having 1-3 heteroatoms independently selected from nitrogen, oxygen, or sulfur. In some embodiments, R is a substituted 3-7 membered saturated or partially unsaturated heterocyclic ring having 1-3 heteroatoms independently selected from nitrogen, oxygen, or sulfur. In some embodiments, R is an unsubstituted 3-7 membered saturated or partially unsaturated heterocyclic ring having 1-3 heteroatoms independently selected from nitrogen, oxygen, or sulfur.

In certain embodiments, R is an optionally substituted 5-7 membered partially unsaturated monocyclic ring having 1-3 heteroatoms independently selected from nitrogen, oxygen, or sulfur. In certain embodiments, R is an optionally substituted 5-6 membered partially unsaturated monocyclic ring having 1-3 heteroatoms independently selected from nitrogen, oxygen, or sulfur. In certain embodiments, R is an optionally substituted 5-membered partially unsaturated monocyclic ring having 1-3 heteroatoms independently selected from nitrogen, oxygen, or sulfur. Exemplary R groups include but are not limited to optionally substituted dihydroimidazolyl, dihydrothiazolyl, dihydrooxazolyl, or oxazolinyl. In certain embodiments, R is an optionally substituted 6-membered partially unsaturated monocyclic ring having 1-3 heteroatoms independently selected from nitrogen, oxygen, or sulfur. Exemplary R groups include but are not limited to optionally substituted dihydropyridinyl, tetrahydropyridinyl, dihydropyrimidinyl, tetrahydropyrimidinyl, dihydropyrazinyl, tetrohydropyrazinyl, dihydrotriazinyl, tetrahydrotriazinyl, dihydrodioxinyl, dihydrooxathiinyl, dihydrooxazinyl, dihydrodithiine, dihydrothiazine, dioxinyl, oxathiinyl, oxazinyl, dithiinyl, or thiazinyl. In certain embodiments, R is an optionally substituted 7-membered partially unsaturated monocyclic ring having 1-3 heteroatoms independently selected from nitrogen, oxygen, or sulfur. Exemplary R groups include but are not limited to optionally substituted azepiyl, oxepinyl, thiepinyl, diazepinyl, oxazepinyl, thiazepinyl, triazepinyl, oxadiazepinyl, thiadiazepinyl, dihydroazepiyl, dihydrooxepinyl, dihydrothiepinyl, dihydrodiazepinyl, dihydrooxazepinyl, dihydrothiazepinyl, dihydrotriazepinyl, dihydrooxadiazepinyl, dihydrothiadiazepinyl, tetrahydroazepiyl, tetrahydrooxepinyl, tetrahydrothiepinyl, tetrahydrodiazepinyl, tetrahydrooxazepinyl, tetrahydrothiazepinyl, tetrahydrotriazepinyl, tetrahydrooxadiazepinyl, or tetrahydrothiadiazepinyl.

In some embodiments, R is an optionally substituted 3-membered heterocyclic ring having one heteroatom selected from nitrogen, oxygen or sulfur. Exemplary R groups include but are not limited to optionally substituted aziridinyl, thiiranyl or oxiranyl. In some embodiments, R is optionally substituted 4-membered heterocyclic ring having 1-3 heteroatoms independently selected from nitrogen, oxygen, or sulfur. Exemplary R groups include but are not limited to optionally substituted azetidinyl, oxetanyl, thietanyl, oxazetidinyl, thiazetidinyl, or diazetidinyl. In some embodiments, R is optionally substituted 5-membered heterocyclic ring having 1-3 heteroatoms independently selected from nitrogen, oxygen, or sulfur. Exemplary R groups include but are not limited to optionally substituted pyrrolidinyl, tetrahydrofuranyl, tetrahydrothienyl, oxazolidinyl, dioxolanyl, oxathiolanyl, thiazolidinyl, dithiolanyl, imidazolidinyl, isothiazolidinyl, pyrazolidinyl, isoxazolidinyl, isothiazolidinyl, triazolidinyl, oxadiazolidinyl, thiadiazolidinyl, oxadiazolidinyl, dioxazolidinyl, oxathiazolidinyl, thiadiazolidinyl or dithiazolidinyl. In some embodiments, R is optionally substituted 6-membered heterocyclic ring having 1-3 heteroatoms independently selected from nitrogen, oxygen, or sulfur. Exemplary R groups include but are not limited to optionally substituted piperidinyl, tetrahydropyranyl, tetrahydrothiopyranyl, piperazinyl, morpholinyl, thiomorpholinyl, dithianyl, dioxanyl, oxathianyl, triazinanyl, oxadiazinanyl, thiadiazinanyl, dithiazinanyl, dioxazinanyl, oxathiazinanyl, oxadithianyl, trioxanyl, dioxathianyl or trithianyl. In some embodiments, R is optionally substituted 7-membered heterocyclic ring having 1-3 heteroatoms independently selected from nitrogen, oxygen, or sulfur. Exemplary R groups include but are not limited to optionally substituted azepanyl, oxepanyl, thiepanyl, diazepanyl, oxazepanyl, thiazepanyl, dioxepanyl, oxathiepanyl, dithiepanyl, triazepanyl, oxadiazepanyl, thiadiazepanyl, dioxazepanyl, oxathiazepanyl, dithiazepanyl, trioxepanyl, dioxathiepanyl, oxadithiepanyl or trithiepanyl.

In certain embodiments, R is optionally substituted oxiranyl, oxetanyl, tetrahydrofuranyl, tetrahydropyranyl, oxepaneyl, aziridineyl, azetidineyl, pyrrolidinyl, piperidinyl, azepanyl, thiiranyl, thietanyl, tetrahydrothienyl, tetrahydrothiopyranyl, thiepanyl, dioxolanyl, oxathiolanyl, oxazolidinyl, imidazolidinyl, thiazolidinyl, dithiolanyl, dioxanyl, morpholinyl, oxathianyl, piperazinyl, thiomorpholinyl, dithianyl, dioxepanyl, oxazepanyl, oxathiepanyl, dithiepanyl, diazepanyl, dihydrofuranonyl, tetrahydropyranonyl, oxepanonyl, pyrolidinonyl, piperidinonyl, azepanonyl, dihydrothiophenonyl, tetrahydrothiopyranonyl, thiepanonyl, oxazolidinonyl, oxazinanonyl, oxazepanonyl, dioxolanonyl, dioxanonyl, dioxepanonyl, oxathiolinonyl, oxathianonyl, oxathiepanonyl, thiazolidinonyl, thiazinanonyl, thiazepanonyl, imidazolidinonyl, tetrahydropyrimidinonyl, diazepanonyl, imidazolidinedionyl, oxazolidinedionyl, thiazolidinedionyl, dioxolanedionyl, oxathiolanedionyl, piperazinedionyl, morpholinedionyl, thiomorpholinedionyl, tetrahydropyranyl, tetrahydrofuranyl, morpholinyl, thiomorpholinyl, piperidinyl, piperazinyl, pyrrolidinyl, tetrahydrothienyl, or tetrahydrothiopyranyl.

In some embodiments, R is an optionally substituted 7-10 membered bicyclic saturated or partially unsaturated heterocyclic ring having 1-5 heteroatoms independently selected from nitrogen, oxygen, or sulfur. In some embodiments, R is optionally substituted indolinyl. In some embodiments, R is optionally substituted isoindolinyl. In some embodiments, R is optionally substituted 1,2,3,4-tetrahydroquinolinyl. In some embodiments, R is optionally substituted 1,2,3,4-tetrahydroisoquinolinyl. In some embodiments, R is an optionally substituted azabicyclo[3.2.1]octanyl.

In some embodiments, R is an optionally substituted 8-10 membered bicyclic heteroaryl ring having 1-5 heteroatoms independently selected from nitrogen, oxygen, or sulfur.

In some embodiments, R is an optionally substituted 5,6-fused heteroaryl ring having 1-5 heteroatoms independently selected from nitrogen, oxygen, or sulfur. In some embodiments, R is an optionally substituted 5,6-fused heteroaryl ring having 1-4 heteroatoms independently selected from nitrogen, oxygen, or sulfur. In some embodiments, R is an optionally substituted 5,6-fused heteroaryl ring having 1-3 heteroatoms independently selected from nitrogen, oxygen, or sulfur. In some embodiments, R is an optionally substituted 5,6-fused heteroaryl ring having two heteroatoms independently selected from nitrogen, oxygen, or sulfur. In some embodiments, R is optionally substituted 1,4-dihydropyrrolo[3,2-b]pyrrolyl, 4H-furo[3,2-b]pyrrolyl, 4H-thieno[3,2-b]pyrrolyl, furo[3,2-b]furanyl, thieno[3,2-b]furanyl, thieno[3,2-b]thienyl, 1H-pyrrolo[1,2-a]imidazolyl, pyrrolo[2,1-b)]oxazolyl or pyrrolo[2,1-b]thiazolyl. In some embodiments, R is an optionally substituted 5,6-fused heteroaryl ring having three heteroatoms independently selected from nitrogen, oxygen, or sulfur. In some embodiments, R is optionally substituted dihydropyrroloimidazolyl, 1H-furoimidazolyl, 1H-thienoimidazolyl, furooxazolyl, furoisoxazolyl, 4H-pyrrolooxazolyl, 4H-pyrroloisoxazolyl, thienooxazolyl, thienoisoxazolyl, 4H-pyrrolothiazolyl, furothiazolyl, thienothiazolyl, 1H-imidazoimidazolyl, imidazooxazolyl or imidazo[5,1-b]thiazolyl. In some embodiments, R is an optionally substituted 5,6-fused heteroaryl ring having four heteroatoms independently selected from nitrogen, oxygen, or sulfur. In some embodiments, R is an optionally substituted 5,6-fused heteroaryl ring having five heteroatoms independently selected from nitrogen, oxygen, or sulfur.

In some embodiments, R is an optionally substituted 5,6-fused heteroaryl ring having 1-5 heteroatoms independently selected from nitrogen, oxygen, or sulfur. In other embodiments, R is an optionally substituted 5,6-fused heteroaryl ring having 1-2 heteroatoms independently selected from nitrogen, oxygen, or sulfur. In certain embodiments, R is an optionally substituted 5,6-fused heteroaryl ring having one heteroatom independently selected from nitrogen, oxygen, or sulfur. In some embodiments, R is optionally substituted indolyl. In some embodiments, R is optionally substituted benzofuranyl. In some embodiments, R is optionally substituted benzo[b]thienyl. In certain embodiments, R is an optionally substituted 5,6-fused heteroaryl ring having two heteroatoms independently selected from nitrogen, oxygen, or sulfur. In some embodiments, R is optionally substituted azaindolyl. In some embodiments, R is optionally substituted benzimidazolyl. In some embodiments, R is optionally substituted benzothiazolyl. In some embodiments, R is optionally substituted benzoxazolyl. In some embodiments, R is an optionally substituted indazolyl. In certain embodiments, R is an optionally substituted 5,6-fused heteroaryl ring having three heteroatoms independently selected from nitrogen, oxygen, or sulfur. In some embodiments, R is optionally substituted oxazolopyridiyl, thiazolopyridinyl or imidazopyridinyl. In certain embodiments, R is an optionally substituted 5,6-fused heteroaryl ring having four heteroatoms independently selected from nitrogen, oxygen, or sulfur. In some embodiments, R is optionally substituted purinyl, oxazolopyrimidinyl, thiazolopyrimidinyl, oxazolopyrazinyl, thiazolopyrazinyl, imidazopyrazinyl, oxazolopyridazinyl, thiazolopyridazinyl or imidazopyridazinyl. In certain embodiments, R is an optionally substituted 5,6-fused heteroaryl ring having five heteroatoms independently selected from nitrogen, oxygen, or sulfur.

In certain embodiments, R is an optionally substituted 6,6-fused heteroaryl ring having 1-5 heteroatoms independently selected from nitrogen, oxygen, or sulfur. In some embodiments, R is an optionally substituted 6,6-fused heteroaryl ring having 1-2 heteroatoms independently selected from nitrogen, oxygen, or sulfur. In other embodiments, R is an optionally substituted 6,6-fused heteroaryl ring having one heteroatom selected from nitrogen, oxygen, or sulfur. In some embodiments, R is optionally substituted quinolinyl. In some embodiments, R is optionally substituted isoquinolinyl. In some embodiments, R is an optionally substituted 6,6-fused heteroaryl ring having two heteroatoms independently selected from nitrogen, oxygen, or sulfur. In some embodiments, R is optionally substituted quinazolinyl, phthalazinyl, quinoxalinyl or naphthyridinyl. In some embodiments, R is an optionally substituted 6,6-fused heteroaryl ring having three heteroatoms independently selected from nitrogen, oxygen, or sulfur. In some embodiments, R is optionally substituted pyridopyrimidinyl, pyridopyridazinyl, pyridopyrazinyl, or benzotriazinyl. In some embodiments, R is an optionally substituted 6,6-fused heteroaryl ring having four heteroatoms independently selected from nitrogen, oxygen, or sulfur. In some embodiments, R is optionally substituted pyridotriazinyl, pteridinyl, pyrazinopyrazinyl, pyrazinopyridazinyl, pyridazinopyridazinyl, pyrimidopyridazinyl or pyrimidopyrimidinyl. In some embodiments, R is an optionally substituted 6,6-fused heteroaryl ring having five heteroatoms independently selected from nitrogen, oxygen, or sulfur.

In some embodiments, R is optionally substituted heterobiaryl wherein each heteroaryl group is independently an optionally substituted group selected from a 5-6 membered monocyclic heteroaryl ring having 1-4 heteroatoms independently selected from nitrogen, oxygen, or sulfur, or an 8-10 membered bicyclic heteroaryl ring having 1-5 heteroatoms independently selected from nitrogen, oxygen, or sulfur. In some embodiments, R is optionally substituted heterobiaryl wherein each aryl group is an optionally substituted 8-10 membered bicyclic heteroaryl ring having 1-5 heteroatoms independently selected from nitrogen, oxygen, or sulfur.

In some embodiments, two R groups on the same atom are optionally taken together with the atom to which they are attached to form an optionally substituted 3-10 membered, saturated, partially unsaturated, or aryl ring having, in addition to the atom to which they are attached, 0-4 heteroatoms independently selected from nitrogen, oxygen, or sulfur. In some embodiments, two R groups on the same carbon atom are optionally taken together with the carbon atom to form an optionally substituted 3-10 membered, saturated, partially unsaturated, or aryl ring having 0-4 heteroatoms independently selected from nitrogen, oxygen, or sulfur. In some embodiments, two R groups on the same nitrogen atom are optionally taken together with the nitrogen atom to form an optionally substituted 3-10 membered, saturated, partially unsaturated, or aryl ring having 1-5 heteroatoms independently selected from nitrogen, oxygen, or sulfur. In some embodiments, two R groups on the same sulfur atom are optionally taken together with the sulfur atom to form an optionally substituted 3-10 membered, saturated, partially unsaturated, or aryl ring having 1-5 heteroatoms independently selected from nitrogen, oxygen, or sulfur. In some embodiments, two R groups on the same oxygen atom are optionally taken together with the oxygen atom to form an optionally substituted 3-10 membered, saturated, partially unsaturated, or aryl ring having 1-5 heteroatoms independently selected from nitrogen, oxygen, or sulfur. In some embodiments, two R groups on the same phosphorus atom are optionally taken together with the phosphorus atom to form an optionally substituted 3-10 membered, saturated, partially unsaturated, or aryl ring having, in addition to the phosphorus atom, 0-4 heteroatoms independently selected from nitrogen, oxygen, or sulfur. In some embodiments, three R groups on the same phosphorus atom, e.g., the three R groups of a phosphine ligand having the structure of P(R)₃, are taken together with the phosphorus atom to form an optionally substituted 3-10 membered, saturated or partially unsaturated, monocyclic, bicyclic or polycyclic ring having, in addition to the phosphorus atom, 0-4 heteroatoms independently selected from nitrogen, oxygen, or sulfur.

A compound of formula I possesses properties that cannot be readily achieved through known catalysts. Among other things, a compound of formula I, having two different R⁴ and R⁵, provides new and better ways for adjusting ligand properties that are important for promoting reactions, including but not limited to steric effects, and/or electron donating/accepting properties of the ligands.

The present invention, among other things, recognizes that provided compounds are particularly challenging to prepare. In some embodiments, a person having ordinary skill in the art, when using known methods in the art, cannot obtain a provided compound in satisfactory yields and/or purity. Stereogenic-at-metal Mo and/or W compounds, such as the monoaryloxide pyrrolide (MAP) compounds, are generally prepared through protonation of the corresponding bispyrrolide compounds using Ar^{b}OH, wherein Ar^{b}O— is the aryloxide ligand ((a) Ibrahem, I; Yu, M.; Schrock, R. R.; Hoveyda, A. H. J. Am. Chem. Soc. 2009, 131, 3844. (b) Flook, M. M.; Jiang, A. J.; Schrock, R. R.; Müller, P.; Hoveyda, A. H. J. Am. Chem. Soc. 2009, 131, 7962. (c) Jiang, A. J.; Zhao, Y.; Schrock, R. R.; Hoveyda, A. H. J. Am. Chem. Soc. 2009, 131, 16630. (d) Flook, M. M.; Gerber, L. C. H.; Debelouchina, G. T.; Schrock, R. R. Macromolecules 2010, 43, 7515. (e) Flook, M. M.; Ng, V. W. L.; Schrock, R. R. J. Am. Chem. Soc. 2011, 133, 1784. (f) Meek, S. J.; O'Brien, R. V.; Llaveria, J.; Schrock, R. R.; Hoveyda, A. H. Nature 2011, 471, 461. (g) Marinescu, S. C.; Schrock, R. R.; Müller, P.; Takase, M. K.; Hoveyda, A. H. Organometallics 2011, 30, 1780. (h) Yu, M.; Ibrahem, I.; Hasegawa, M.; Schrock, R. R.; Hoveyda, A. H. J. Am. Chem. Soc. 2012, 134, 2788. (i) Townsend, E. M.; Schrock, R. R.; Hoveyda, A. H. J. Am. Chem. Soc. 2012, 134, 11334. (j) Wang, C.; Yu, M.; Kyle, A. F.; Jacubec, P.; Dixon, D. J.; Schrock, R. R.; Hoveyda, A. H. Chem. Eur. J. 2013, 19, 2726. (k) Wang, C.; Haeffner, F.; Schrock, R. R.; Hoveyda, A. H. Angew. Chem. Int. Ed. 2013, 52, 1939. (1) Flook, M. M.; Borner, J.; Kilyanek, S.; Gerber, L. C. H.; Schrock, R. R. Organometallics 2012, 31, 6231). However, it is not known, and cannot be readily predicted, whether two different alkoxide, aryloxide, or carboxylate ligands can be introduced to the same Mo or W complexes to produce a compound of enough purity for promoting metathesis reactions; known Mo or W complexes with two monodentate alkoxide or aryloxide ligands generally have the same two monodentate alkoxide or aryloxide ligands. When R⁵ is —N(R′)₂, or R, protonation of bispyrrolide compounds does not work efficiently. Accordingly, the present invention provides new methods for preparing a compound of formula I.

In some embodiments, the present invention provides a method for preparing a compound of formula I, comprising:

a) providing a compound of formula II:

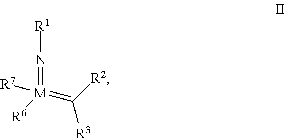

wherein:

each of R⁶ and R⁷ is independently optionally substituted pyrrolide; and

each of R′, R² and R³ is independently as defined above and described herein;

b) reacting the compound of formula II with a compound having the structure of R⁴H, or its salt thereof, to provide a compound of formula III:

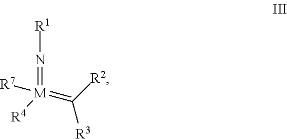

wherein each variable is independently as defined above and described herein;

c) reacting the compound of formula III with a compound having the structure of R⁵H, or its salt thereof, to provide a compound of formula I.

In some embodiments, the present invention provides a method for preparing a compound of formula I, comprising:

a) providing a compound of formula II:

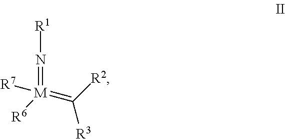

wherein each variable is independently as defined above and described herein;

b) reacting the compound of formula II with a compound having the structure of R⁵H, or its salt thereof, to provide a compound of formula III′:

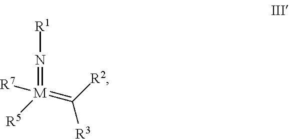

wherein each variable is independently as defined above and described herein;

c) reacting the compound of formula III′ with a compound having the structure of R⁴H, or its salt thereof, to provide a compound of formula I.

In some embodiments, the present invention provides a method for preparing a compound of formula I, comprising:

a) providing a compound of formula II′:

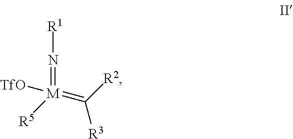

wherein each variable is independently as defined above and described herein;

b) reacting the compound of formula II′ with a compound having the structure of R⁴H, or its salt thereof, to provide a compound of formula I.

In some embodiments, R⁵H is R′OH. In some embodiments, R⁵H is R′OH, wherein R′ is not hydrogen. In some embodiments, R⁵H is R′C(O)OH.

It is surprisingly found, as exemplified by the examples described herein, that the above methods provided a compound of formula I with good yield and purity for promoting metathesis reactions. In some embodiments, R⁵ is —OR′.

In some embodiments, the present invention provides a method for preparing a compound of formula I, comprising:

a) providing a compound of formula IV:

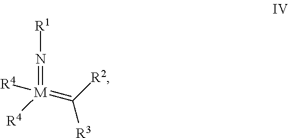

wherein each variable is independently as defined above and described herein;

b) reacting the compound of formula IV with a compound having the structure of R⁵H, or its salt thereof, to provide a compound of formula I.

In some embodiments, a salt of R⁵H is used in step b. In some embodiments, R⁵Li is used in step b.

In some embodiments, R⁴ is —O—C(R^{t})₂—R′, wherein —C(R^{t})₂—R′ is optionally substituted with one or more —F. In some embodiments, R⁴ is —OCMe(CF₃)₂.

In some embodiments, R⁵ H is R′OH. In some embodiments, R⁵H is R′OH, wherein R′ is not hydrogen. In some embodiments, a salt of R′OH is R′OLi. In some embodiments, R⁵ H is H′OC(O)R′. In some embodiments, a salt of R′OH is R′C(O)OLi. In some embodiments, R⁵H is HN(R′)₂. In some embodiments, a salt of R⁵H is LiN(R′)₂. In some embodiments, R⁵H is H₂NR′. In some embodiments, a salt of R⁵H is LiNHR′. In some embodiments, R⁵H is H₂NR″. In some embodiments, a salt of R⁵H is LiNHR“.

In some embodiments, R⁵ is R”, and a salt of R⁵H is used in step b. In some embodiments, R⁵ is R″, and R⁵Li is used in step b.

In some embodiments, a provided method suppresses or eliminates undesirable competitive deprotonation of the alkylidene ligand, which leads to low yield and/or impurities difficult to remove.

Exemplary compounds of formula I include but are not limited to: Mo(NAd)(CHCMe₂Ph)(OHIPT)(OCMe₃), Mo(NAr)(CHCMe₂Ph)(OR_{F6})(OHMT), Mo(NAr′)(CHCMe₂Ph)(OR_{F6})(OHMT), Mo(NAr^{iPr})(CHCMe₂Ph)(OR_{F6})(OHMT), Mo(NAd)(CHCMe₂Ph)(OR_{F6})(OHMT), Mo(NAd)(CHCMe₂Ph)(OR_{F6})[N(H)HMT)], Mo(NAr′)(CHCMe₂Ph)(OR_{F6})[N(H)HMT)], Mo(NAr^{iPr})(CHCMe₂Ph)(OR_{F6})[N(H)HMT)], Mo(NAr)(CHCMe₂Ph)(OR_{F6})[N(H)HMT)], Mo(NAr)(CHCMe₂Ph)(OR_{F6})(O₂CTer_{Me}),

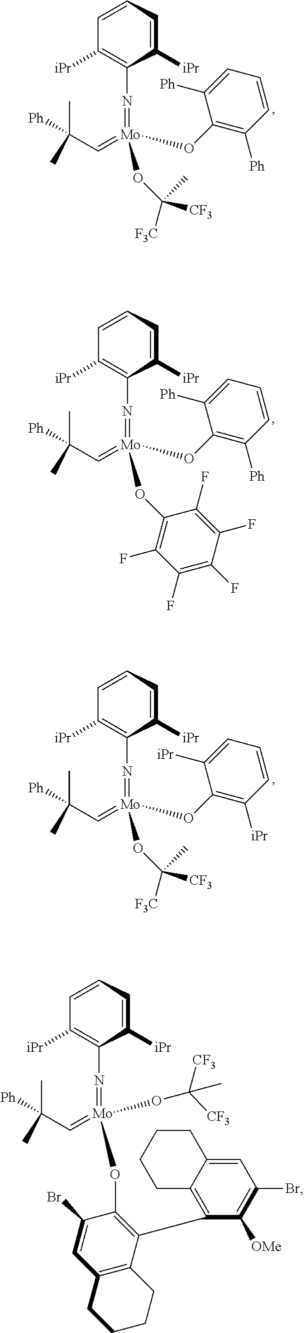

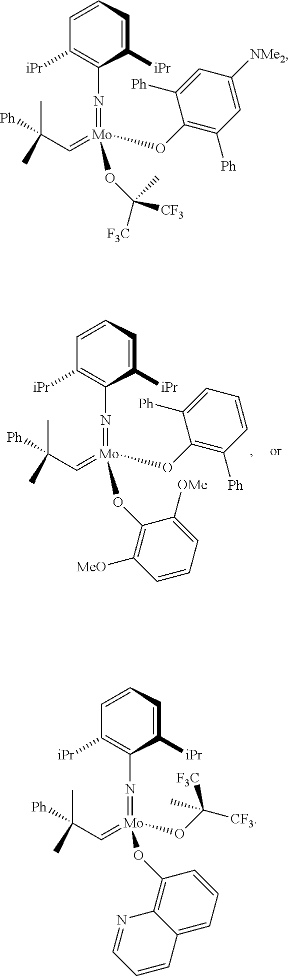

In some embodiments, an exemplary compound is Mo(NAd)(CHCMe₂Ph)(OR_{F6})(HMT), Mo(NAr^{m})(CHCMe₂Ph)(OR_{F6})(HMT), Mo(NAr′)(CHCMe₂Ph)(OR_{F6})(HMT), Mo(NAr)(CHCMe₂Ph)(OR_{F6})(HMT), Mo(NAd)(CHCMe₂Ph)(OR_{F6})(TIPT), Mo(NAr′)(CHCMe₂Ph)(OR_{F6})(TIPT), Mo(NAr)(CHCMe₂Ph)(OR_{F6})(TIPT), or Mo(NAd)(CHCMe₂Ph)(OR_{F9})(HMT).

Exemplary compounds of formula II include but are not limited to: Mo(NAd)(CHCMe₂Ph)(MesPyr)₂, Mo(NAd)(CHCMe₂Ph)(2-CNPyr)₂, Mo(NC₆F₅)(CHCMe₂Ph)(Me₂Pyr)₂,

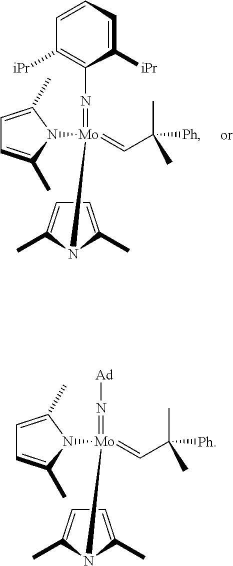

In some embodiments, a compound of formula II′ is Mo(NAd)(CHCMe₂Ph)(OTf)(OHIPT).

Exemplary compounds of formula III include but are not limited to:

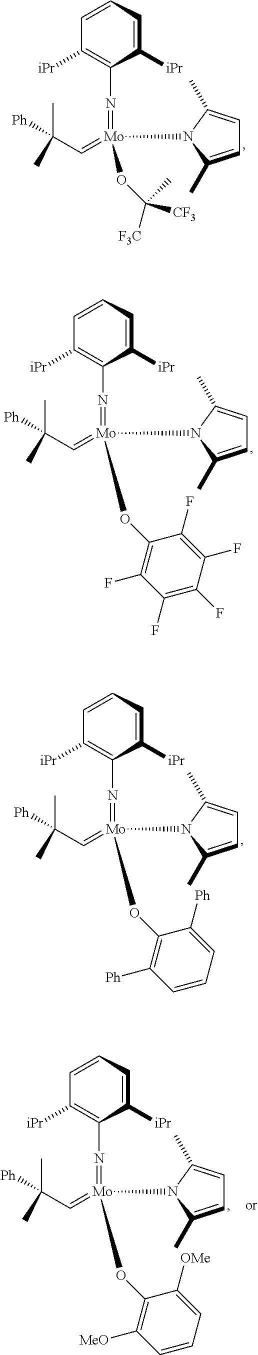

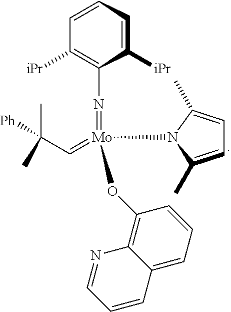

Exemplary compounds of formula III′ include but are not limited to:

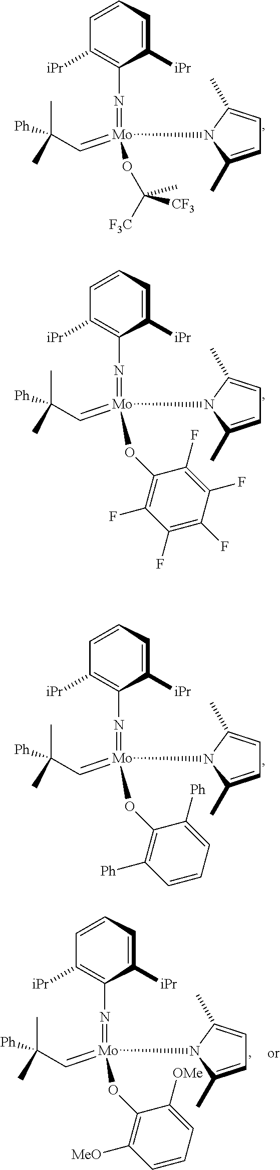

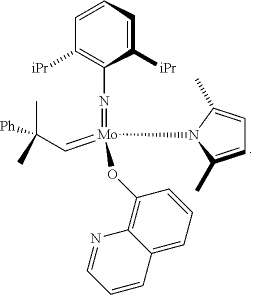

Exemplary compounds of formula IV include but are not limited to Mo(NR)(CHCMe₂Ph)(OR_{F6})₂, Li[Mo(NAd)(CHCMe₂Ph)(OiPr^{F6})₃] and Mo(NAd)(CHCMe₂Ph) (OR_{F9})₂

Exemplary R⁴H include but are not limited to Me₃COH, (CF₃)₂CHOH, (CF₃)₃COH, LiOHIPT, (CF₃)₂MeCOH, pentafluorophenol, 2,6-diphenylphenol, 8-hydroxyquinoline, 2,6-dimethoxyphenyl, 2,6-diphenyl-4-dimethylaminophenol, 3-bromo-1-(3-bromo-2-methoxy-5,6,7,8-tetrahydronaphthalen-1-yl)-5,6,7,8-tetrahydronaphthalen-2-ol.

Exemplary R⁵H include but are not limited to Me₃COH, (CF₃)₂CHOH, (CF₃)₃COH, LiOHIPT, 2,6-diphenylphenol, pentafluorophenol, 8-hydroxyquinoline, 2,6-dimethoxyphenol, 2,6-diphenyl-4-dimethylaminophenol, 3-bromo-1-(3-bromo-2-methoxy-5,6,7,8-tetrahydronaphthalen-1-yl)-5,6,7,8-tetrahydronaphthalen-2-ol, HO₂CTer_{Me}.

In some other embodiments, the present invention provides methods for metathesis reactions. In some embodiments, a provided method comprises providing a compound provided by this invention. In some embodiments, a provided method produces a product with unexpected selectivity. For example, ROMP of dicarbomethoxynorbornadiene (DCMNBD) promoted by a compound of formula I, in some embodiments, produces polymers with cis, isotactic selectivity. In some embodiments, ROMP promoted by a compound of formula I produces polymers with cis, syndiotactic selectivity.

In some embodiments, the present invention provides a method for performing a metathesis reaction, comprising providing a compound having the structure of formula I. In some embodiments, a metathesis reaction is olefin metathesis. In some embodiments, a metathesis reaction is ring-opening metathesis polymerization (ROMP). In some embodiments, a metathesis reaction is ROMP, and a product is produced with cis, isotactic selectivity. In some embodiments, a metathesis reaction is ROMP, and a product is produced with cis, syndiotactic selectivity.

In some embodiments, a metathesis reaction is homocoupling olefin metathesis. In some embodiments, a homocoupling olefin metathesis produces the product with Z selectivity. In some embodiments, a homocoupling olefin metathesis produces the product with E selectivity. In some embodiments, a substrate of a homocoupling olefin metathesis is a terminal olefin. In some embodiments, a substrate is 1-hexene. In some embodiments, a substrate is 1-octene.

In some embodiments, a metathesis reaction is ring-closing metathesis (RCM). In some embodiments, a substrate of an RCM reaction is diallyl ether.

In some embodiments, the present invention provides a method for ring-opening metathesis polymerization (ROMP), comprising providing a compound having the structure of formula I, wherein the ROMP polymer product has greater than about 50% isotactic structure.

In some embodiments, the present invention provides a method for ring-opening metathesis polymerization (ROMP), comprising providing a compound having the structure of formula I, wherein the ROMP polymer product has greater than about 50% syndiotactic structure.

In some embodiments, an ROMP product is greater than about 50% isotactic. In some embodiments, an ROMP product is greater than about 60% isotactic. In some embodiments, an ROMP product is greater than about 70% isotactic. In some embodiments, an ROMP product is greater than about 80% isotactic. In some embodiments, an ROMP product is greater than about 85% isotactic. In some embodiments, an ROMP product is greater than about 90% isotactic. In some embodiments, an ROMP product is greater than about 91% isotactic. In some embodiments, an ROMP product is greater than about 92% isotactic. In some embodiments, an ROMP product is greater than about 93% isotactic. In some embodiments, an ROMP product is greater than about 94% isotactic. In some embodiments, an ROMP product is greater than about 95% isotactic. In some embodiments, an ROMP product is greater than about 96% isotactic. In some embodiments, an ROMP product is greater than about 97% isotactic. In some embodiments, an ROMP product is greater than about 98% isotactic. In some embodiments, an ROMP product is greater than about 99% isotactic.

In some embodiments, an ROMP product is greater than about 50% syndiotactic. In some embodiments, an ROMP product is greater than about 60% syndiotactic. In some embodiments, an ROMP product is greater than about 70% syndiotactic. In some embodiments, an ROMP product is greater than about 80% syndiotactic. In some embodiments, an ROMP product is greater than about 85% syndiotactic. In some embodiments, an ROMP product is greater than about 90% syndiotactic. In some embodiments, an ROMP product is greater than about 91% syndiotactic. In some embodiments, an ROMP product is greater than about 92% syndiotactic. In some embodiments, an ROMP product is greater than about 93% syndiotactic. In some embodiments, an ROMP product is greater than about 94% syndiotactic. In some embodiments, an ROMP product is greater than about 95% syndiotactic. In some embodiments, an ROMP product is greater than about 96% syndiotactic. In some embodiments, an ROMP product is greater than about 97% syndiotactic. In some embodiments, an ROMP product is greater than about 98% syndiotactic. In some embodiments, an ROMP product is greater than about 99% syndiotactic.

In some embodiments, an ROMP product is greater than about 50% cis. In some embodiments, an ROMP product is greater than about 60% cis. In some embodiments, an ROMP product is greater than about 70% cis. In some embodiments, an ROMP product is greater than about 80% cis. In some embodiments, an ROMP product is greater than about 85% cis. In some embodiments, an ROMP product is greater than about 90% cis. In some embodiments, an ROMP product is greater than about 91% cis. In some embodiments, an ROMP product is greater than about 92% cis. In some embodiments, an ROMP product is greater than about 93% cis. In some embodiments, an ROMP product is greater than about 94% cis. In some embodiments, an ROMP product is greater than about 95% cis. In some embodiments, an ROMP product is greater than about 96% cis. In some embodiments, an ROMP product is greater than about 97% cis. In some embodiments, an ROMP product is greater than about 98% cis. In some embodiments, an ROMP product is greater than about 99% cis.

In some embodiments, an ROMP product is greater than about 50% trans. In some embodiments, an ROMP product is greater than about 60% trans. In some embodiments, an ROMP product is greater than about 70% trans. In some embodiments, an ROMP product is greater than about 80% trans. In some embodiments, an ROMP product is greater than about 85% trans. In some embodiments, an ROMP product is greater than about 90% trans. In some embodiments, an ROMP product is greater than about 91% trans. In some embodiments, an ROMP product is greater than about 92% trans. In some embodiments, an ROMP product is greater than about 93% trans. In some embodiments, an ROMP product is greater than about 94% trans. In some embodiments, an ROMP product is greater than about 95% trans. In some embodiments, an ROMP product is greater than about 96% trans. In some embodiments, an ROMP product is greater than about 97% trans. In some embodiments, an ROMP product is greater than about 98% trans. In some embodiments, an ROMP product is greater than about 99% trans.

In some embodiments, an ROMP product is greater than about 50% cis and greater than about 50% isotactic. In some embodiments, an ROMP product is greater than about 60% cis and greater than about 50% isotactic. In some embodiments, an ROMP product is greater than about 70% cis and greater than about 50% isotactic. In some embodiments, an ROMP product is greater than about 80% cis and greater than about 50% isotactic. In some embodiments, an ROMP product is greater than about 85% cis and greater than about 50% isotactic. In some embodiments, an ROMP product is greater than about 90% cis and greater than about 50% isotactic. In some embodiments, an ROMP product is greater than about 95% cis and greater than about 50% isotactic. In some embodiments, an ROMP product is greater than about 98% cis and greater than about 50% isotactic. In some embodiments, an ROMP product is greater than about 90% cis and greater than about 60% isotactic. In some embodiments, an ROMP product is greater than about 90% cis and greater than about 70% isotactic. In some embodiments, an ROMP product is greater than about 90% cis and greater than about 80% isotactic. In some embodiments, an ROMP product is greater than about 90% cis and greater than about 90% isotactic. In some embodiments, an ROMP product is greater than about 95% cis and greater than about 90% isotactic. In some embodiments, an ROMP product is greater than about 98% cis and greater than about 90% isotactic. In some embodiments, an ROMP product is greater than about 95% cis and greater than about 95% isotactic.

In some embodiments, an ROMP product is greater than about 50% cis and greater than about 50% syndiotactic. In some embodiments, an ROMP product is greater than about 60% cis and greater than about 50% syndiotactic. In some embodiments, an ROMP product is greater than about 70% cis and greater than about 50% syndiotactic. In some embodiments, an ROMP product is greater than about 80% cis and greater than about 50% syndiotactic. In some embodiments, an ROMP product is greater than about 85% cis and greater than about 50% syndiotactic. In some embodiments, an ROMP product is greater than about 90% cis and greater than about 50% syndiotactic. In some embodiments, an ROMP product is greater than about 95% cis and greater than about 50% syndiotactic. In some embodiments, an ROMP product is greater than about 98% cis and greater than about 50% syndiotactic. In some embodiments, an ROMP product is greater than about 90% cis and greater than about 60% syndiotactic. In some embodiments, an ROMP product is greater than about 90% cis and greater than about 70% syndiotactic. In some embodiments, an ROMP product is greater than about 90% cis and greater than about 80% syndiotactic. In some embodiments, an ROMP product is greater than about 90% cis and greater than about 90% syndiotactic. In some embodiments, an ROMP product is greater than about 95% cis and greater than about 90% syndiotactic. In some embodiments, an ROMP product is greater than about 98% cis and greater than about 90% syndiotactic. In some embodiments, an ROMP product is greater than about 95% cis and greater than about 95% syndiotactic.

In some embodiments, a metathesis reaction using a compound of the present invention produces a polymer wherein the polymer is >50% cis, >50% syndiotactic. In some embodiments, a metathesis reaction using a compound of the present invention produces a polymer wherein the polymer is >60% cis, >60% syndiotactic. In some embodiments, a metathesis reaction using a compound of the present invention produces a polymer wherein the polymer is >70% cis, >70% syndiotactic. In some embodiments, a metathesis reaction using a compound of the present invention produces a polymer wherein the polymer is 80% cis, >80% syndiotactic. In some embodiments, a metathesis reaction using a compound of the present invention produces a polymer wherein the polymer is >90% cis, 90% syndiotactic. In some embodiments, a metathesis reaction using a compound of the present invention produces a polymer wherein the polymer is >95% cis, 90% syndiotactic. In some embodiments, a metathesis reaction using a compound of the present invention produces a polymer wherein the polymer is >99% cis, 90% syndiotactic. In some embodiments, a metathesis reaction using a compound of the present invention produces a polymer wherein the polymer is >90% cis, >95% syndiotactic. In some embodiments, a metathesis reaction using a compound of the present invention produces a polymer wherein the polymer is >95% cis, >95% syndiotactic. In some embodiments, a metathesis reaction using a compound of the present invention produces a polymer wherein the polymer is >99% cis, >90% syndiotactic. In some embodiments, a metathesis reaction using a compound of the present invention produces a polymer wherein the polymer is >99% cis, >95% syndiotactic. In some embodiments, a metathesis reaction using a compound of the present invention produces a polymer wherein the polymer is >99% cis, >97% syndiotactic. In some embodiments, a metathesis reaction using a compound of the present invention produces a polymer wherein the polymer is >99% cis, >99% syndiotactic.

In some embodiments, an ROMP product is greater than about 50% cis and greater than about 50% isotactic. In some embodiments, an ROMP product is greater than about 60% cis and greater than about 50% isotactic. In some embodiments, an ROMP product is greater than about 70% cis and greater than about 50% isotactic. In some embodiments, an ROMP product is greater than about 80% cis and greater than about 50% isotactic. In some embodiments, an ROMP product is greater than about 85% cis and greater than about 50% isotactic. In some embodiments, an ROMP product is greater than about 90% cis and greater than about 50% isotactic. In some embodiments, an ROMP product is greater than about 95% cis and greater than about 50% isotactic. In some embodiments, an ROMP product is greater than about 98% cis and greater than about 50% isotactic. In some embodiments, an ROMP product is greater than about 90% cis and greater than about 60% isotactic. In some embodiments, an ROMP product is greater than about 90% cis and greater than about 70% isotactic. In some embodiments, an ROMP product is greater than about 90% cis and greater than about 80% isotactic. In some embodiments, an ROMP product is greater than about 90% cis and greater than about 90% isotactic. In some embodiments, an ROMP product is greater than about 95% cis and greater than about 90% isotactic. In some embodiments, an ROMP product is greater than about 98% cis and greater than about 90% isotactic. In some embodiments, an ROMP product is greater than about 95% cis and greater than about 95% isotactic.

In some embodiments, a metathesis reaction using a compound of the present invention produces a polymer wherein the polymer is >50% cis, >50% isotactic. In some embodiments, a metathesis reaction using a compound of the present invention produces a polymer wherein the polymer is >60% cis, >60% isotactic. In some embodiments, a metathesis reaction using a compound of the present invention produces a polymer wherein the polymer is >70% cis, >70% isotactic. In some embodiments, a metathesis reaction using a compound of the present invention produces a polymer wherein the polymer is 80% cis, >80% isotactic. In some embodiments, a metathesis reaction using a compound of the present invention produces a polymer wherein the polymer is >90% cis, 90% isotactic. In some embodiments, a metathesis reaction using a compound of the present invention produces a polymer wherein the polymer is >95% cis, 90% isotactic. In some embodiments, a metathesis reaction using a compound of the present invention produces a polymer wherein the polymer is >99% cis, 90% isotactic. In some embodiments, a metathesis reaction using a compound of the present invention produces a polymer wherein the polymer is >90% cis, >95% isotactic. In some embodiments, a metathesis reaction using a compound of the present invention produces a polymer wherein the polymer is >95% cis, >95% isotactic. In some embodiments, a metathesis reaction using a compound of the present invention produces a polymer wherein the polymer is >99% cis, >90% isotactic. In some embodiments, a metathesis reaction using a compound of the present invention produces a polymer wherein the polymer is >99% cis, >95% isotactic. In some embodiments, a metathesis reaction using a compound of the present invention produces a polymer wherein the polymer is >99% cis, >97% isotactic. In some embodiments, a metathesis reaction using a compound of the present invention produces a polymer wherein the polymer is >99% cis, >99% isotactic.

Some embodiments may provide the ability to selectively synthesize, via a metathesis reaction, products having a Z or E configuration about a double bond. In some embodiments, a method of the present invention provides the ability to synthesize compounds comprising a Z-olefin. In some embodiments, such methods are useful when applied to a wide range of olefin substrates, including those having sterically small or large groups adjacent the olefin. In some embodiments, the substrate olefins are terminal olefins.

In some embodiments, the present invention provides a method for Z-selective metathesis reactions. In some embodiments, a provided method produces a double bond in a Z:E ratio greater than about 1:1, greater than about 2:1, greater than about 3:1, greater than about 4:1, greater than about 5:1, greater than about 6:1, greater than about 7:1, greater than about 8:1, greater than about 9:1, greater than about 95:5, greater than about 96:4, greater than about 97:3, greater than about 98:2, or, in some cases, greater than about 99:1, as determined using methods described herein (e.g., HPLC or NMR). In some cases, about 100% of the double bond produced in the metathesis reaction may have a Z configuration. The Z or cis selectivity may also be expressed as a percentage of product formed. In some cases, the product may be greater than about 50% Z, greater than about 60% Z, greater than about 70% Z, greater than about 80% Z, greater than about 90% Z, greater than about 95% Z, greater than about 96% Z, greater than about 97% Z, greater than about 98% Z, greater than about 99% Z, or, in some cases, greater than about 99.5% Z.

In some embodiments, a method of the present invention provides the ability to synthesize compounds comprising a E-olefin. In some embodiments, the present invention provides a method for E-selective metathesis reactions. In some embodiments, a provided method produces a double bond in a E:Z ratio greater than about 1:1, greater than about 2:1, greater than about 3:1, greater than about 4:1, greater than about 5:1, greater than about 6:1, greater than about 7:1, greater than about 8:1, greater than about 9:1, greater than about 95:5, greater than about 96:4, greater than about 97:3, greater than about 98:2, or, in some cases, greater than about 99:1, as determined using methods described herein (e.g., HPLC or NMR). In some cases, about 100% of the double bond produced in the metathesis reaction may have a E configuration. The E or trans selectivity may also be expressed as a percentage of product formed. In some cases, the product may be greater than about 50% E, greater than about 60% E, greater than about 70% E, greater than about 80% E, greater than about 90% E, greater than about 95% E, greater than about 96% E, greater than about 97% E, greater than about 98% E, greater than about 99% E, or, in some cases, greater than about 99.5% E.

### Conditions

In some embodiments, a ligand is provided in a molar ratio of about 10:1, 9:1, 8:1, 7:1, 6:1, 5:1, 4:1, 3:1, 2:1, or 1:1 relative to the metal. In some embodiments, a ligand is provided in a molar ratio of about 0.9:1, 0.8:1, 0.7:1, 0.6:1, 0.5:1, 0.4:1, 0.3:1, 0.2:1, or 0.1:1 relative to the metal. In certain embodiments, a ligand is provided in a molar ratio of about 1:1 relative to the metal. One of skill in the art will appreciate that the optimal molar ratio of ligand to metal will depend on, inter alia, whether the ligand is mono- or polydentate. In some embodiments, a ligand or ligand precursor having the structure of formula I is provided in a molar ratio of about 1:1 to Mo or W.

Suitable conditions for performing provided methods generally employ one or more solvents. In certain embodiments, one or more organic solvents are used. Examples of such organic solvents include, but are not limited to, hydrocarbons such as benzene, toluene, and pentane, halogenated hydrocarbons such as dichloromethane and chloroform, or polar aprotic solvents, such as ethereal solvents including ether, tetrahydrofuran (THF), or dioxanes, or protic solvents, such as alcohols, or mixtures thereof. In certain embodiments, one or more solvents are deuterated.

In some embodiments, a single solvent is used. In certain embodiments, a solvent is benzene. In certain embodiments, a solvent is ether. In some embodiments, a solvent is a nitrile. In some embodiments, a solvent is acetonitrile.

In some embodiments, mixtures of two or more solvents are used, and in some cases may be preferred to a single solvent. In certain embodiments, the solvent mixture is a mixture of an ethereal solvent and a hydrocarbon. Exemplary such mixtures include, for instance, an ether/benzene mixture. Solvent mixtures may be comprised of equal volumes of each solvent or may contain one solvent in excess of the other solvent or solvents. In certain embodiments wherein a solvent mixture is comprised of two solvents, the solvents may be present in a ratio of about 20:1, about 10:1, about 9:1, about 8:1, about 7:1, about 6:1, about 5:1, about 4:1, about 3:1, about 2:1, or about 1:1. In certain embodiments wherein a solvent mixture comprises an ethereal solvent and a hydrocarbon, the solvents may be present in a ratio of about 20:1, about 10:1, about 9:1, about 8:1, about 7:1, about 6:1, about 5:1, about 4:1, about 3:1, about 2:1, or about 1:1 ethereal solvent: hydrocarbon. In certain embodiments, the solvent mixture comprises a mixture of ether and benzene in a ratio of about 5:1. One of skill in the art would appreciate that other solvent mixtures and/or ratios are contemplated herein, that the selection of such other solvent mixtures and/or ratios will depend on the solubility of species present in the reaction (e.g., substrates, additives, etc.), and that experimentation required to optimized the solvent mixture and/or ratio would be routine in the art and not undue.

Suitable conditions, in some embodiments, employ ambient temperatures. In some embodiments, a suitable temperature is about 15° C., about 20° C., about 25° C., or about 30° C. In some embodiments, a suitable temperature is from about 15° C. to about 25° C. In certain embodiments, a suitable temperature is about 20° C., 21° C., 22° C., 23° C., 24° C., or 25° C.

In certain embodiments, a provided method is performed at elevated temperature. In some embodiments, a suitable temperature is from about 25° C. to about 110° C. In certain embodiments, a suitable temperature is from about 40° C. to about 100° C., from about 50° C. to about 100° C., from about 60° C. to about 100° C., from about 70° C. to about 100° C., from about 80° C. to about 100° C., or from about 90° C. to about 100° C. In some embodiments, a suitable temperature is about 80° C. In some embodiments, a suitable temperature is about 30° C. In some embodiments, a suitable temperature is about 40° C. In some embodiments, a suitable temperature is about 50° C. In some embodiments, a suitable temperature is about 60° C. In some embodiments, a suitable temperature is about 70° C. In some embodiments, a suitable temperature is about 80° C. In some embodiments, a suitable temperature is about 90° C. In some embodiments, a suitable temperature is about 100° C. In some embodiments, a suitable temperature is about 110° C.

In certain embodiments, a provided method is performed at temperature lower than ambient temperatures. In some embodiments, a suitable temperature is from about −100° C. to about 10° C. In certain embodiments, a suitable temperature is from about −80° C. to about 0° C. In certain embodiments, a suitable temperature is from about −70° C. to about 10° C. In certain embodiments, a suitable temperature is from about −60° C. to about 10° C. In certain embodiments, a suitable temperature is from about −50° C. to about 10° C. In certain embodiments, a suitable temperature is from about −40° C. to about 10° C. In certain embodiments, a suitable temperature is or from about −30° C. to about 10° C. In some embodiments, a suitable temperature is below 0° C. In some embodiments, a suitable temperature is about −100° C. In some embodiments, a suitable temperature is about −90° C. In some embodiments, a suitable temperature is about −80° C. In some embodiments, a suitable temperature is about −70° C. In some embodiments, a suitable temperature is about −60° C. In some embodiments, a suitable temperature is about −50° C. In some embodiments, a suitable temperature is about −40° C. In some embodiments, a suitable temperature is about −30° C. In some embodiments, a suitable temperature is about −20° C. In some embodiments, a suitable temperature is about −10° C. In some embodiments, a suitable temperature is about 0° C. In some embodiments, a suitable temperature is about 10° C.

In some embodiments, a provided method is performed at different temperatures. In some embodiments, temperature changes in a provided method. In some embodiments, a provided method involves temperature increase from a lower suitable temperature to a higher suitable temperature. In some embodiments, a provided method comprises temperature increase from about −80° C., about −70° C., about −60° C., about −50° C., about −40° C., about −30° C., about −20° C., about −10° C., and about 0° C. to about 0° C., about 10° C., about 20° C., ambient temperature, about 22° C., about 25° C., about 30° C., about 40° C., about 50° C., about 60° C., about 70° C., about 80° C., about 90° C., about 100° C. and about 110° C. In some embodiments, a provided method comprises temperature increase from about −30° C. to 22° C. In some embodiments, a provided method comprises temperature decrease from a higher suitable temperature to a lower suitable temperature. In some embodiments, a provided method comprises temperature increase from about 110° C., about 100° C., about 90° C., about 80° C., about 70° C., about 60° C., about 50° C., about 40° C., about 30° C., about 25° C., about 22° C., ambient temperature, about 20° C., about 10° C., and about 0° C. to about 0° C., about −10° C., about −20° C., about −30° C., about −40° C., about −50° C., about −60° C., about −70° C., about −80° C., about −90° C., and about −100° C.

Suitable conditions typically involve reaction times of about 1 minute to about one or more days. In some embodiments, the reaction time ranges from about 0.5 hour to about 20 hours. In some embodiments, the reaction time ranges from about 0.5 hour to about 15 hours. In some embodiments, the reaction time ranges from about 1.0 hour to about 12 hours. In some embodiments, the reaction time ranges from about 1 hour to about 10 hours. In some embodiments, the reaction time ranges from about 1 hour to about 8 hours. In some embodiments, the reaction time ranges from about 1 hour to about 6 hours. In some embodiments, the reaction time ranges from about 1 hour to about 4 hours. In some embodiments, the reaction time ranges from about 1 hour to about 2 hours. In some embodiments, the reaction time ranges from about 2 hours to about 8 hours. In some embodiments, the reaction time ranges from about 2 hours to about 4 hours. In some embodiments, the reaction time ranges from about 2 hours to about 3 hours. In certain embodiments, the reaction time is about 1, 2, 3, 4, 5, 6, 7, 8, 9, 10, 11, or 12, 24, 48, 96 or 120 hours. In certain embodiments, the reaction time is about 1 hour. In certain embodiments, the reaction time is about 2 hours. In certain embodiments, the reaction time is about 3 hours. In certain embodiments, the reaction time is about 4 hours. In certain embodiments, the reaction time is about 5 hours. In certain embodiments, the reaction time is about 6 hours. In some embodiments, the reaction time is about 12 hours. In some embodiments, the reaction time is about 24 hours. In some embodiments, the reaction time is about 48 hours. In some embodiments, the reaction time is about 96 hours. In some embodiments, the reaction time is about 120 hours. In certain embodiments, the reaction time is less than about 1 hour. In certain embodiments, the reaction time is about 5, 10, 15, 20, 25, 30, 35, 40, 45, 50, or 55 minutes. In some embodiments, the reaction time is about 5 minutes. In some embodiments, the reaction time is about 10 minutes. In some embodiments, the reaction time is about 15 minutes. In some embodiments, the reaction time is about 20 minutes. In some embodiments, the reaction time is about 25 minutes. In some embodiments, the reaction time is about 30 minutes. In some embodiments, the reaction time is about 35 minutes. In some embodiments, the reaction time is about 40 minutes. In some embodiments, the reaction time is about 100 minutes. In some embodiments, the reaction time is about 110 minutes. In some embodiments, the reaction time is about 200 minutes. In some embodiments, the reaction time is about 300 minutes. In some embodiments, the reaction time is about 400 minutes.

In some embodiments, a provided metal complex compound, e.g. a compound of formula I, or an active catalyst formed from a provided compound, is stable under metathesis conditions. In some embodiments, a provided compound, or an active catalyst formed from a provided compound, decomposes under metathesis conditions. In some embodiments, a provided compound, or an active catalyst formed from a provided compound, decomposes under metathesis conditions within about 1 hour. In some embodiments, a provided compound, or an active catalyst formed from a provided compound, decomposes under metathesis conditions within about 2 hours. In some embodiments, a provided compound, or an active catalyst formed from a provided compound, decomposes under metathesis conditions within about 6 hours. In some embodiments, a provided compound, or an active catalyst formed from a provided compound, decomposes under metathesis conditions within about 12 hours. In some embodiments, a provided compound, or an active catalyst formed from a provided compound, decomposes under metathesis conditions within about 24 hours. In some embodiments, a provided compound, or an active catalyst formed from a provided compound, decomposes under metathesis conditions within about 48 hours. In some embodiments, a provided compound, or an active catalyst formed from a provided compound, decomposes under metathesis conditions within about 96 hours.

In some embodiments, a provided method requires an amount of a provided compound (e.g., a metal complex having the structure of formula I) such that the loading is from about 0.001 mol % to about 20 mol % of the provided compound relative to substrate (e.g., a first or second double bond). In certain embodiments, a provided compound is used in an amount of between about 0.001 mol % to about 10 mol %. In certain embodiments, a provided compound is used in an amount of between about 0.001 mol % to about 6 mol %. In certain embodiments, a provided compound is used in an amount of between about 0.001 mol % to about 5 mol %. In certain embodiments, a provided compound is used in an amount of between about 0.001 mol % to about 4 mol %. In certain embodiments, a provided compound is used in an amount of between about 0.001 mol % to about 3 mol %. In certain embodiments, a provided compound is used in an amount of between about 0.001 mol % to about 1 mol %. In certain embodiments, a provided compound is used in an amount of between about 0.001 mol % to about 0.5 mol %. In certain embodiments, a provided compound is used in an amount of between about 0.001 mol % to about 0.2 mol %. In certain embodiments, a provided compound is used in an amount of about 0.001 mol %, 0.002 mol %, 0.005 mol %, 0.01 mol %, 0.02 mol %, 0.03 mol %, 0.04 mol %, 0.05 mol %, 0.1 mol %, 0.2 mol %, 0.5 mol %, 1 mol %, 2 mol %, 3 mol %, 4 mol %, 5 mol %, 6 mol %, 7 mol %, 8 mol %, 9 mol %, or 10 mol %. In some embodiments, a provided compound is used in an amount of about 0.0002% mol. In some embodiments, a provided compound is used in an amount of about 0.01% mol. In some embodiments, a provided compound is used in an amount of about 3% mol.

In some embodiments, a method of the present invention requires an amount of solvent such that the concentration of the reaction is between about 0.01 M and about 1 M. In some embodiments, the concentration of the reaction is between about 0.01 M and about 0.5 M. In some embodiments, the concentration of the reaction is between about 0.01 M and about 0.1 M. In some embodiments, the concentration of the reaction is between about 0.01 M and about 0.05 M. In some embodiments, the concentration of the reaction is about 0.01 M. In some embodiments, the concentration of the reaction is about 0.02 M. In some embodiments, the concentration of the reaction is about 0.03 M. In some embodiments, the concentration of the reaction is about 0.04 M. In some embodiments, the concentration of the reaction is about 0.05 M. In some embodiments, the concentration of the reaction is about 0.1 M. In some embodiments, the concentration of the reaction is about 0.3 M.

In some embodiments, a method of the present invention is performed at ambient pressure. In some embodiments, a method of the present invention is performed at reduced pressure. In some embodiments, a method of the present invention is performed at a pressure of less than about 20 torr. In some embodiments, a method of the present invention is performed at a pressure of less than about 15 torr. In some embodiments, a method of the present invention is performed at a pressure of less than about 10 torr. In some embodiments, a method of the present invention is performed at a pressure of about 9, 8, 7, 6, 5, 4, 3, 2, or 1 torr. In certain embodiments, a method of the present invention is performed at a pressure of about 7 torr. In certain embodiments, a method of the present invention is performed at a pressure of about 1 torr.

In some embodiments, a method of the present invention is performed at increased pressure. In some embodiments, a method of the present invention is performed at greater than about 1 atm. In some embodiments, a method of the present invention is performed at greater than about 2 atm. In some embodiments, a method of the present invention is performed at greater than about 3 atm. In some embodiments, a method of the present invention is performed at greater than about 5 atm. In some embodiments, a method of the present invention is performed at greater than about 10 atm. In some embodiments, a method of the present invention is performed at about 2 atm. In some embodiments, a method of the present invention is performed at about 3 atm. In some embodiments, a method of the present invention is performed at about 5 atm. In some embodiments, a method of the present invention is performed at about 10 atm.

As mentioned above, provided compounds are useful for metathesis reactions. Exemplary such methods and reactions are described below.

It will be appreciated that, in certain embodiments, each variable recited is as defined above and described in embodiments, herein, both singly and in combination.

### EXEMPLIFICATION

The present invention recognizes, among other things, that there is a continuing demand for compounds and methods for metathesis reactions. In some embodiments, the present invention provides novel compounds for metathesis reactions, their preparation methods and use thereof. In some embodiments, the prevent invention provides novel methods for metathesis. Exemplary but non-limiting examples are depicted herein.

### Preparation of New Bispyrrolide Compounds

Mo(NAd)(CHCMe₂Ph)(MesPyr)₂ (1a; MesPyr=2-mesitylpyrrolide, Ad=1-adamantyl) can be prepared in 75% isolated yield by treating Mo(NAd)(CHCMe₂Ph)(OTf)₂(DME) with two equivalents of Li(MesPyr) in diethyl ether. Mo(NAd)(CHCMe₂Ph)(MesPyr)₂ reacts with TPPOH (2,3,5,6-tetraphenylphenol) and Br₂BitetOH (eq 1) readily to yield Mo(NAd)(CHCMe₂Ph)(MesPyr)(OTPP) (2a) and Mo(NAd)(CHCMe₂Ph)(MesPyr)(OBr₂Bitet) (2b) in 80% and 53% yields, respectively. These syntheses of 2a and 2b are typical protonolysis methods. Compound 2b is found as two diastereomers with syn alkylidene proton shifts of 12.47 ppm (R-diastereomer) and 13.14 ppm (S-diastereomer). The S-diastereomer could be isolated in pure form through crystallization from n-pentane. A typical observed ratio of R to S in the crude product mixture is 1:1.

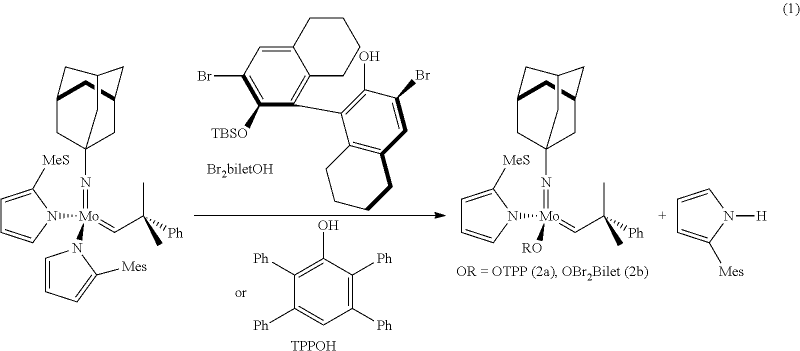

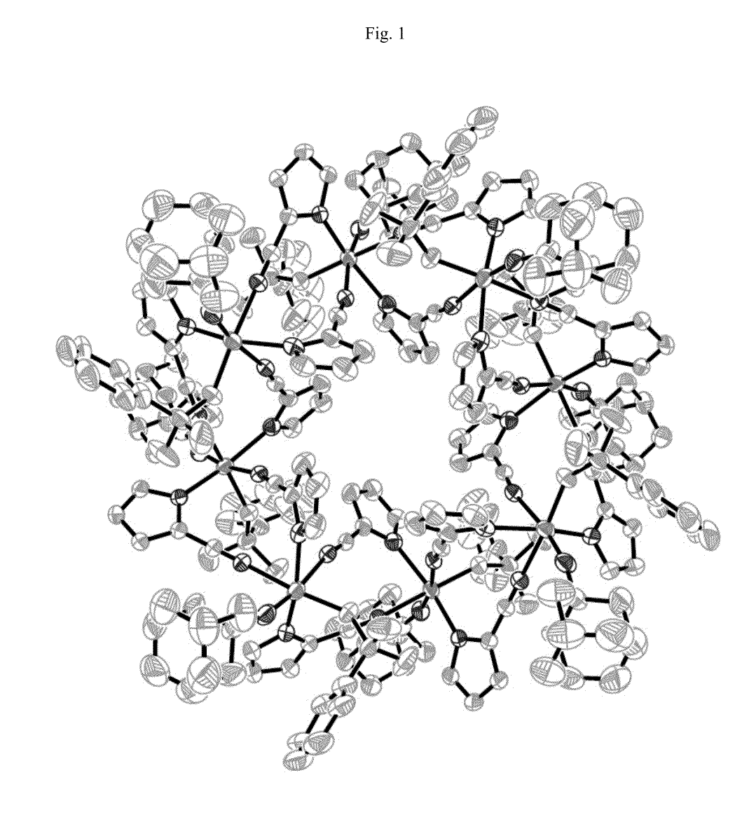

The reaction of Mo(NAd)(CHCMe₂Ph)(OTf)₂(DME) with two equivalents of Li(2-CNPyr) yielded Mo(NAd)(CHCMe₂Ph)(2-CNPyr)₂ (1b) in 55% isolated yield. Alternatively, 1b can be obtained in 76% yield by treating Mo(NAd)(CHCMe₂Ph)(Me₂Pyr)₂ with excess 2-CNPyrH (eq 2). The proton NMR spectrum of 1b typically contains several alkylidene resonances in the range 14-15 ppm, the intensities of which vary from sample to sample. An NMR spectrum of 1b that had been recrystallized from a mixture of THF and pentane contained only a single alkylidene resonance at 14.47 ppm. An X-ray crystallographic study of this sample showed the product to be an octamer (FIG. 1). Without the intention to be limited by theory, we propose that multiple alkylidene resonances arise from other oligomers of Mo(NAd)(CHCMe₂Ph)(2-CNPyr)₂.

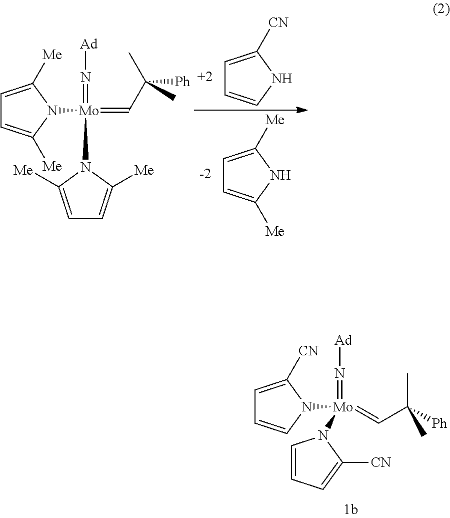

Each molybdenum center in 1b (FIG. 1) exhibits a pseudo-octahedral geometry. The two η¹-pyrrolides are trans to one another and two cyano groups from each of the two adjacent neighboring Mo complexes are coordinated trans to the alkylidene and imido ligands. Eight bispyrrolide units of this type are linked through cyano donor interactions to yield the doughnut-like octameric structure. The bond lengths and angles in any one unit in the octamer are not unusual. It should be noted that Mo(NAr)(CHCMe₂Ph)(NC₄H₄)₂ was found to be a dimer, {Mo(NAr)(syn-CHCMe₂Ph)(η⁵-NC₄H₄)(η¹-NC₄H₄)} {Mo(NAr)(syn-CHCMe₂Ph)(η¹-NC₄H₄)₂}, in which the nitrogen in the η⁵-pyrrolide bound to one Mo behaves as a donor to the other Mo.

Compound 1b reacts with Me₃COH, (CF₃)₂CHOH, and (CF₃)₃COH in C₆D₆ at 22° C. to give the known bisalkoxide complexes exclusively, according to NMR studies.

Formation of Monotriflate Monoaryloxide Complexes and Reactions Thereof

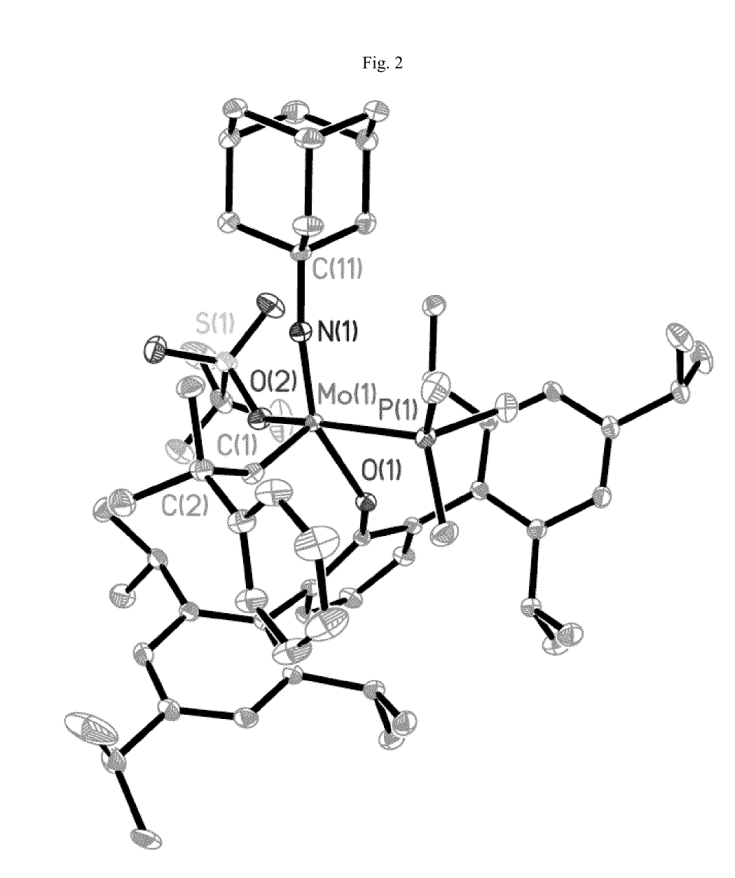

The reaction between Mo(NAd)(CHCMe₂Ph)(OTf)₂(DME) and LiOHIPT in benzene at 80° C. leads to formation of Mo(NAd)(CHCMe₂Ph)(OTf)(OHIPT) (3) in 99% yield (equation 3). Filtration of the reaction mixture and removal of the benzene in vacuo yields 3 as a dark yellow solid that can be employed in subsequent reactions without further purification. Compound 3 shows a single resonance in its ¹⁹F NMR spectrum at δ −75.4 ppm, consistent with the formation of a monotriflate species, while a single syn alkylidene resonance is found at 12.35 ppm (J_{CH}=123 Hz) in its ¹H NMR spectrum. A trimethylphosphine adduct of 3 (3(PMe₃)) was prepared readily and crystals suitable for an X-ray study obtained. As shown in FIG. 2 3(PMe₃) is approximately a square pyramid with the alkylidene in the apical position and PMe₃ coordinated trans to the triflate ligand. The bond distances and angles in 3(PMe₃) are similar to what have been found recently in other PMe₃ adducts of imido alkylidene complexes such as Mo(NAr)(CHCMe₂Ph)(Me₂Pyr)(OBr₂Bitet)(PMe₃) (Marinescu, S. C.; Schrock, R. R.; Li, B.; Hoveyda, A. H. J. Am. Chem. Soc. 2009, 131, 58) and Mo(NAr)(CHCMe₂Ph)(Ph₂Pyr)(OR_{F6})(PMe₃) (Marinescu, S. C.; Singh, R.; Hock, A. S.; Wampler, K. M.; Schrock, R. R.; Müller, P. Organometallics 2008, 27, 6570).

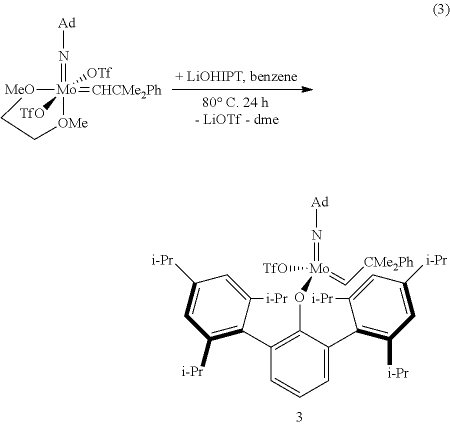

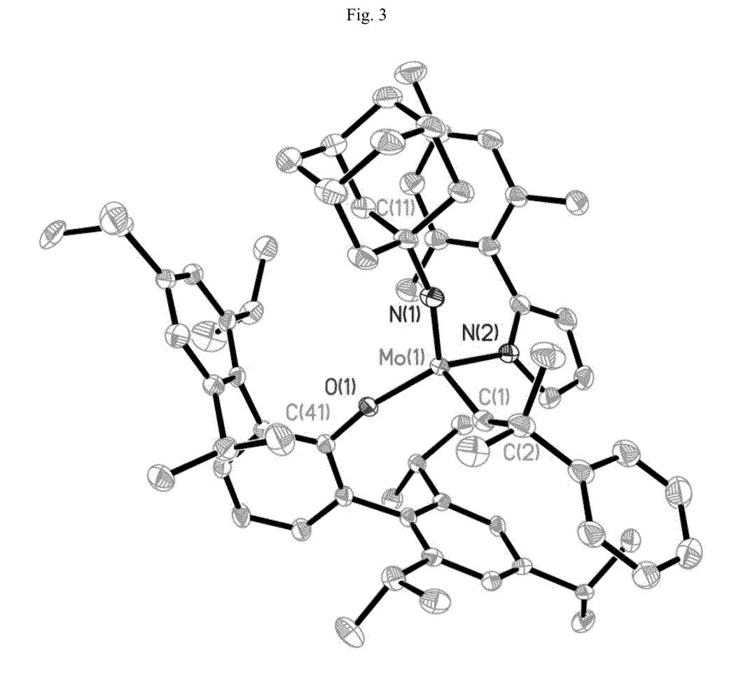

A reaction between one equivalent of sodium 2-mesitylpyrrolide and 3 in benzene (80° C. for 10 h) led to the formation of Mo(NAd)(CHCMe₂Ph)(OHIPT)(2-Mespyr) (2c) in 45% yield (eq 4). A single alkylidene resonance (at 12.25 ppm) with a J_{CH} characteristic of a syn species (120 Hz) is observed in the ¹H NMR spectrum of 2c. A structural study of 2c reveals it to have a slightly distorted tetrahedral geometry in which the mesityl group points away from the sterically demanding OHIPT ligand toward the relatively small adamantylimido ligand (FIG. 3). The imido ligand is bent slightly (Mo(1)-N(1)-C(11)=163.58(11)°).

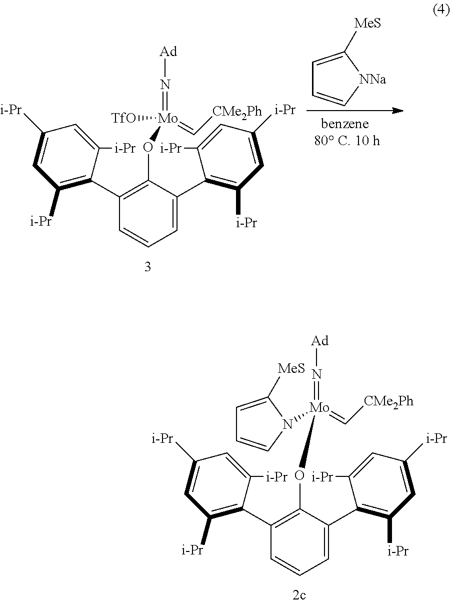

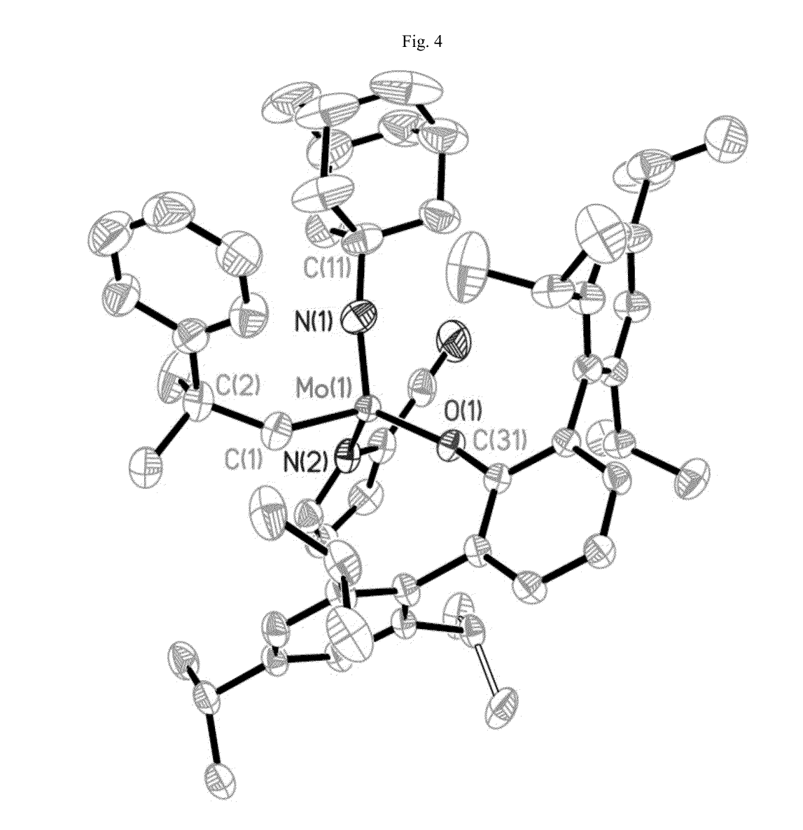

Reaction of 3 with one equivalent of Li(2-CNPyr) in benzene at room temperature gave a complex mixture of products from which Mo(NAd)(CHCMe₂Ph)(2-CNPyr)(OHIPT) (4) could be isolated in 25% yield (equation 5). Formation of free HIPTOH and the relatively low yield, without the intention to be limited by theory, is a consequence of deprotonation of the alkylidene. The ¹H NMR spectrum of pure 4 is straightforward; the syn alkylidene has a J_{CH} of 121 Hz. An X-ray study of 4 confirmed that it is a monomer (FIG. 4). Evidently the steric demands of the HIPTO ligand prevent the cyano group from binding to another Mo center in this circumstance.

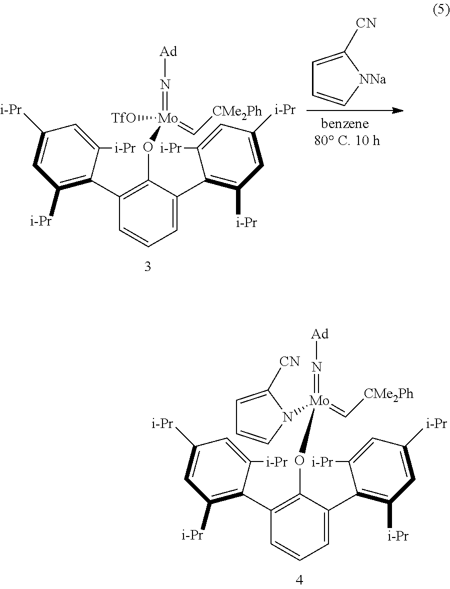

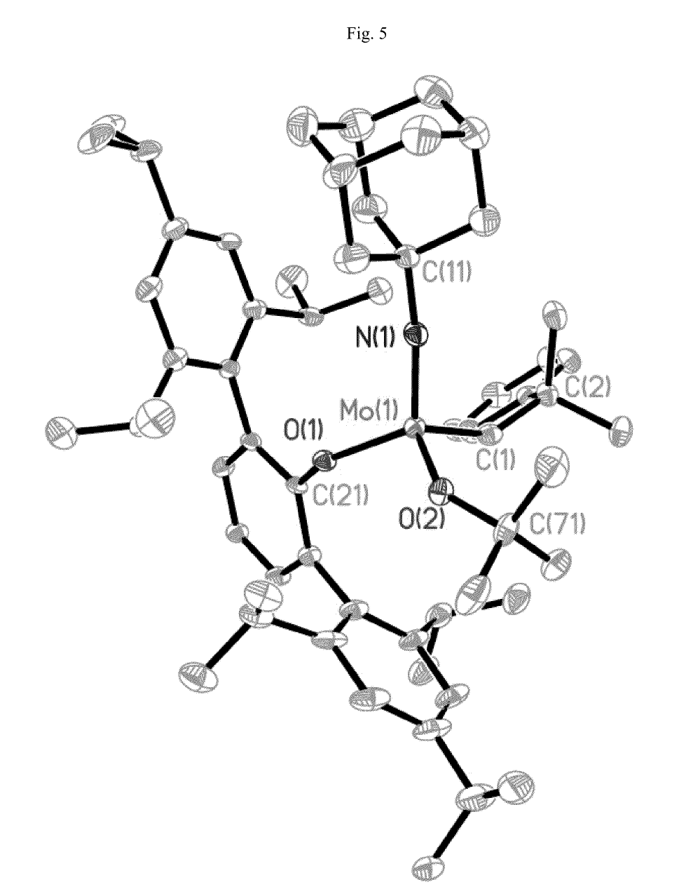

The reaction between one equivalent of LiO-t-Bu and 3 in benzene at room temperature for one day led to the formation of Mo(NAd)(CHCMe₂Ph)(OHIPT)(OCMe₃) (5) in 22% isolated yield (eq 6). While not wishing to be limited by theory, we propose that the low yield again is a consequence, at least in part, of competitive deprotonation of the alkylidene ligand. A single alkylidene resonance (at 11.16 ppm) with J_{CH} characteristic of a syn species (119 Hz) was observed in the ¹H NMR spectrum of 5. A structural study reveals 5 to have the expected tetrahedral geometry (FIG. 5). The Mo(1)-O(2)-C(71) angle)(143.3(2)° and the Mo(1)-O(1)-C(21) angle)(145.2(2)° are essentially identical.

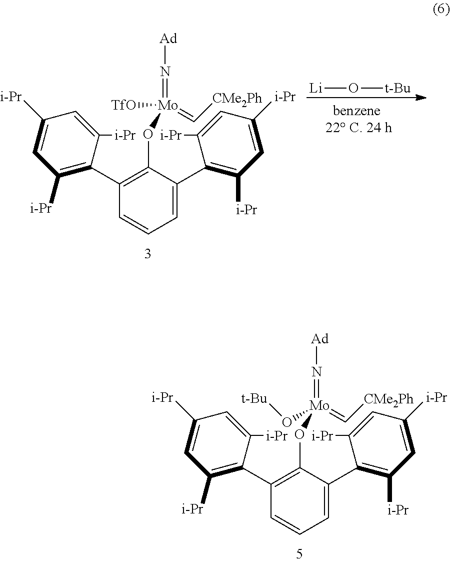

### Synthesis of SAM Complexes from Bishexafluoro-t-Butoxide Complexes

When Mo(NR)(CHCMe₂Ph)(OR_{F6})₂ complexes are treated with one equivalent of LiOHMT, Mo(NR)(CHCMe₂Ph)(OR_{F6})(OHMT) complexes are formed where R=Ar (6a), Ar′ (6b), Ar^{iPr}. (6c), or Ad (6d) in moderate to good yields (43-80%, equation 7). Complexes 6b-6d can be made without formation of any significant byproducts, except in the case of 6a. The proton NMR spectrum of crude of 6a shows that a substantial amount of HMTOH forms, without the intention to be limited by theory, consistent with deprotonation of the alkylidene ligand.

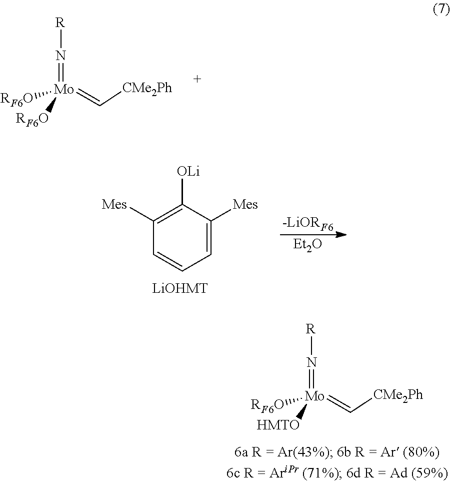

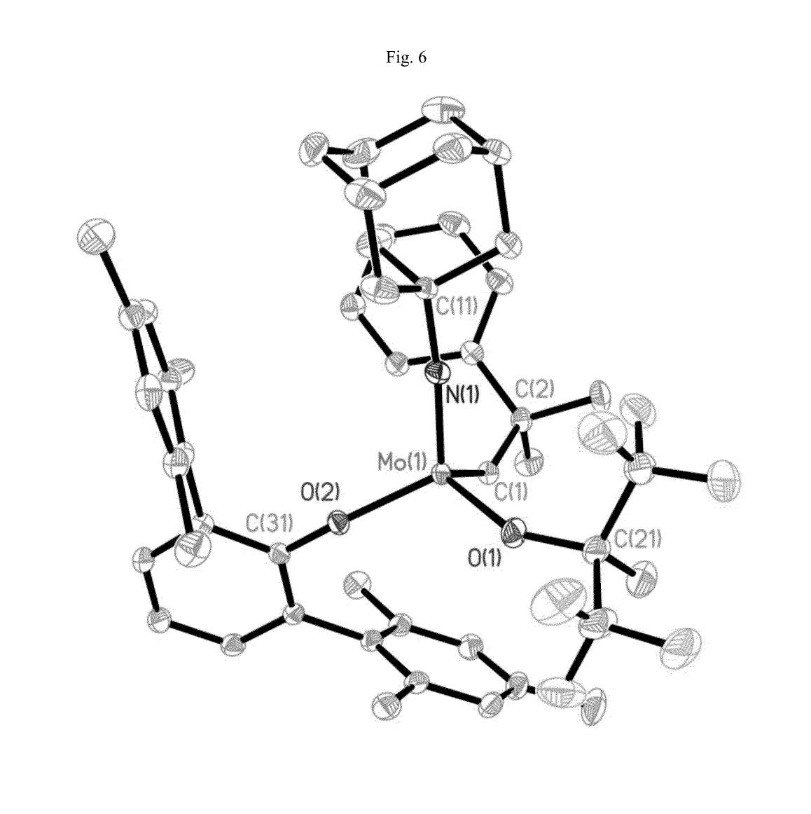

The structure of complex 6d is shown in FIG. 6. The HMTO ligand is oriented so that one of the mesityl groups points toward the imido group while the other points into the COO face of the tetrahedron.

When Mo(NR)(CHCMe₂Ph)(OR_{F6})₂ is treated with one equivalent of LiN(H)HMT at 22° C. in diethyl ether, 7a (R=Ar′) and 7b (R=Ar^{iPr}) could be obtained cleanly (equation 8). Proton NMR spectra of 7a and 7b show only one alkylidene peak (at 11.86 ppm for 7a and 11.72 ppm for 7b) and one NH resonance (at 7.82 ppm for 7a and 7.99 ppm for 7b). The reaction between Mo(NAr)(CHCMe₂Ph)(OR_{F6})₂ and LiN(H)HMT leads to formation of some byproducts. In the case of Mo(NAd)(CHCMe₂Ph)(OR_{F6})₂, substitution was successful, according to proton NMR studies.

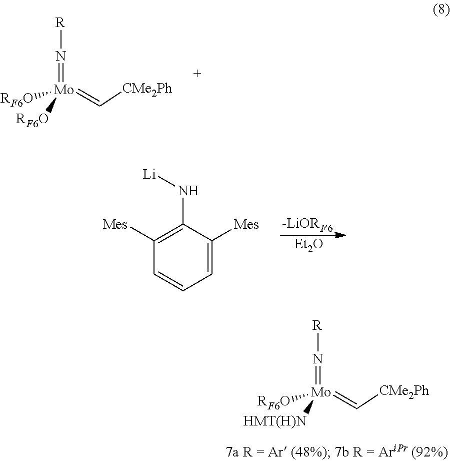

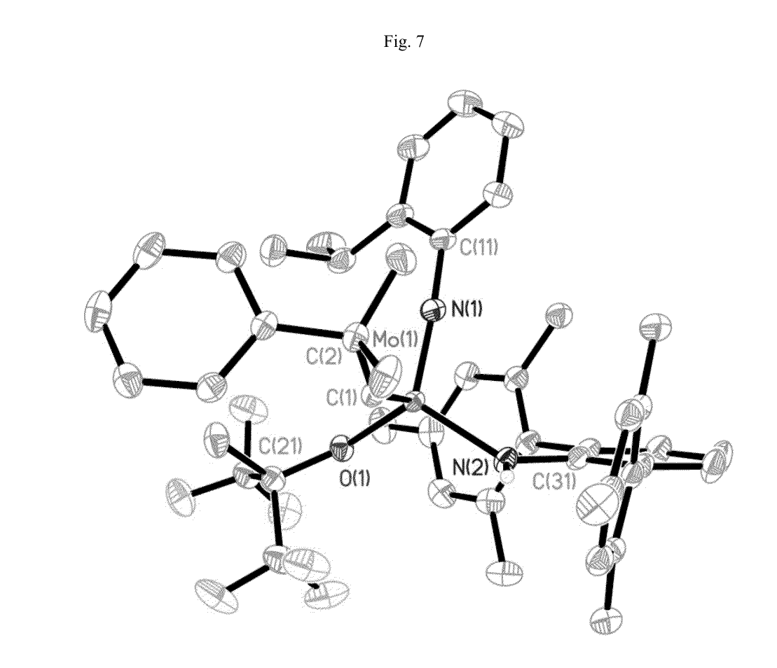

The structure of 7b is shown in FIG. 7 with relevant bond distances and angles listed either in the figure caption or in Table 1. The Mo(1)-N(2) bond distance (1.9950(13) A) is similar to Mo—N_{amido} distances in Mo(NAr)(CHCMe₂Ph)(NPh₂)₂ complexes (2.007(3) and 2.009(3) A) (Sinha, A.; Schrock, R. R.; Müller, P.; Hoveyda, A. H. Organometallics 2006, 25, 4621), but the Mo(1)-N(2)-C(31) bond angle)(133.32(11)° is significantly larger than those of the bisamide complex (118.61(19)° and 117.6(3)°).

When Mo(NAd)(CHCMe₂Ph)(OR_{F6})₂ was treated with one equivalent of LiHMT, Mo(NAd)(CHCMe₂Ph)(OR_{F6})(HMT) (8) was be obtained as a crystalline yellow solid (equation 9). A proton NMR spectrum of 8 reveals the presence of only one product, as determined by the presence of only one alkylidene peak at 10.99 ppm in its ¹H-NMR spectrum and the set of quartets in its ¹⁹F-NMR spectrum.

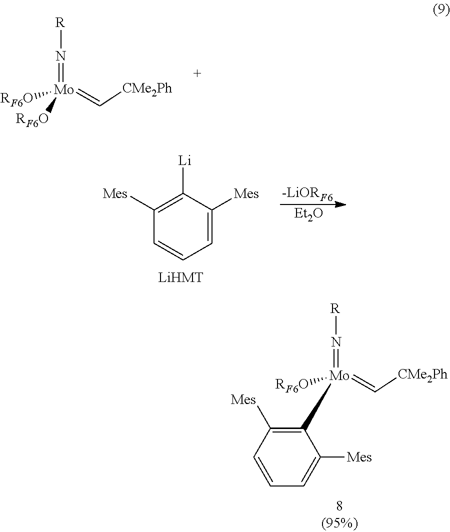

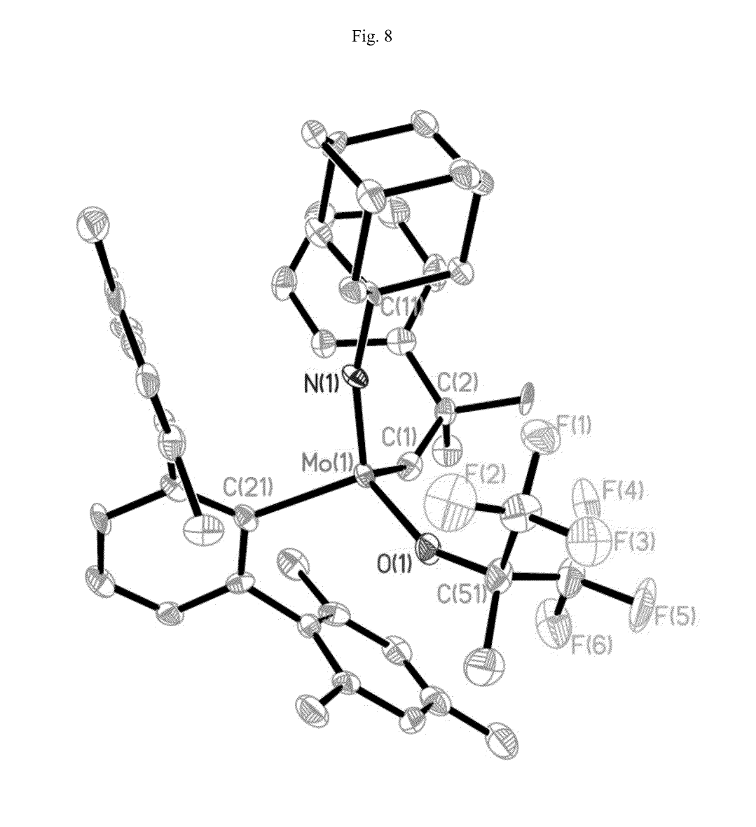

The structure of 8 is shown in FIG. 8. The hexafluoro-t-butoxide ligand is disordered. The Mo(1)-C(21) bond length (2.188(5) Å) is typical of a Mo—C bond length. However, without the intention to be limited by theory, the HMT ligand is considerably more sterically demanding than an OHMT ligand since no heteroatom is present between the Mo and C(21).

Li[Mo(NAd)(CHCMe₂Ph)(OiPr^{F6})₃] and Mo(NAd)(CHCMe₂Ph)(OR_{F9})₂ can also be used (OR_{F9}=OC(CF₃)₃, OiPr^{F6}=OCH(CF₃)₂) in this reaction providing the corresponding products.

TABLE 1
Selected bond lengths (Å) and bond angles (°) in
Mo(NR)(CHR′)(OR″)(X) complexes.

| | 2c | 3(PMe₃) | 4 | 5 | 6d | 7b | 8 |
|---|---|---|---|---|---|---|---|
| Mo═C | 1.8811(16) | 1.8951(16) | 1.883(4) | 1.880(3) | 1.8821(16) | 1.8833(16) | 1.895(5) |
| Mo—X | 2.0551(13) | 1.7413(13) | 2.053(3) | 1.879(2) | 1.9444(12) | 1.9950(13) | 2.188(5) |
| Mo═N | 1.7127(13) | 2.0180(11) | 1.714(3) | 1.713(3) | 1.7028(14) | 1.7261(13) | 1.703(4) |
| Mo—O | 1.909(5) | 2.1721(11) | 1.906(2) | 1.941(2) | 1.9230(11) | 1.9518(11) | 1.943(5) |
| Mo═N—C | 163.58(11) | 168.35(11) | 171.6(3) | 168.73(3) | 169.14(12) | 173.09(12) | 162.1(4) |
| Mo—O—C | 169.3(10) | 139.66(9) | 163.00(19) | 145.2(2) | 154.38(10) | 140.93(10) | 156.2(8) |
| Mo—C—C | 144.53(12) | 147.17(12) | 142.4(3) | 145.6(3) | 141.90(12) | 144.78(12) | 144.7(4) |

ROMP Reactions

ROMP polymerization of 2,3-dicarbomethoxynorbornadiene has been employed to show the activity of certain compounds. Polymerization of DCMNBD with 6a, 6b, 6d, 7a, 7b, and 8, provided poly(DCMNBD) with mix tacticity. The structures of poly(DCMNBD) samples obtained with initiators 6a and 7b were biased toward cis, isotactic, behavior that is unexpected and not readily explicable. The results are presented in Table 2.

TABLE 2
ROMP of 2,3-dicarbomethoxynorbornadiene (DCMNBD).^{a}

| | [Cat] | Eq | |
|---|---|---|---|
| Catalyst | (mM) | DCMNBD | Structure |
| 6a | 4.6 | 50 | >98% cis, |
| Mo(NAr)(CHR′)(OR_{F6})(OHMT) | | | 78% iso |
| 6b | 4.9 | 50 | 95% cis, |
| Mo(NAr′)(CHR′)(OR_{F6})(OHMT) | | | 73% syndio |
| 6c | 4.8 | 50 | 98% cis, |
| Mo(NAr^{iPr})(CHR′)(OR_{F6})(OHMT) | | | 95% syndio |
| 6d | 4.7 | 50 | 90% cis, |
| Mo(NAd)(CHR′)(OR_{F6})(OHMT) | | | 76% syndio |
| 7a | 4.9 | 50 | 95% cis, |
| Mo(NAr′)(CHR′)(OR_{F6})[N(H)HMT] | | | 71% syndio |
| 7b | 4.8 | 50 | 90% cis, |
| Mo(NAr^{iPr})(CHR′)(OR_{F6})[N(H)HMT] | | | 54% iso |
| 8 | 4.8 | 100 | 83% cis, |
| Mo(NAd)(CHR′)(OR_{F6})(HMT) | | | 91% syndio^{b} |

^{a}R′ = CMe₂Ph.
^{b}Five days were required to reach full conversion.

Experimental

General.

All manipulations of air and moisture sensitive materials were conducted under a nitrogen atmosphere in a Vacuum Atmospheres glovebox or on a dual-manifold Schlenk line. All glassware, including NMR tubes, was dried in an oven prior to use. Ether, pentane, toluene, dichloromethane, toluene, and benzene were degassed with dinitrogen, passed through activated alumina columns, and stored over 4 Å Linde-type molecular sieves. Dimethoxyethane was distilled in vacuo from a dark purple solution of sodium benzophenone ketyl and degassed three times by a freeze-pump-thaw procedure. Deuterated solvents were dried over 4 Å Linde-type molecular sieves prior to use. Proton and carbon NMR spectra were acquired using 500 MHz Varian and 400 MHz Bruker spectrometers at room temperature, are reported as parts per million relative to tetramethylsilane, and are referenced to the residual ¹H/¹³C resonances of the deuterated solvent (¹H: CDCl₃, δ 7.26; C₆D₆, δ 7.16. ¹³C: CDCl₃, δ 77.23; C₆D₆, δ 128.39). Compounds 2-CNPyrH (Adamczyk, M.; Reddy, R. E. Tetrahedron Letters 1995, 36, 7983), 2-MesPyrH ((a) Reith, R. D.; Mankad, N. P.; Calimano, E.; Sadighi, J. P. Org. Lett. 2004, 6, 3981. (b) Adamczyk, M.; Reddy, R. E. Tetrahedron Letters 1995, 36, 7983), HMTOH (Stanciu, C.; Olmstead, M. M.; Phillips, A. D.; Stender, M.; Power, P. P. Eur. J. Inorg. Chem. 2003, 3495), HMTNH₂ ((a) Gavenonis, J.; Tilley, T. D. J. Am. Chem. Soc. 2002, 124, 8536. (b) Gavenonis, J.; Tilley, T. D. Organometallics 2004, 23, 31), HMTLi (Schiemenz, B.; Power, P. P. Organometallics 1996, 15, 958), DCMNBD (Tabor, D. C.; White, F. H.; Collier, L. W.; Evans, S. A. J. Org. Chem. 1983, 48, 1638), Mo(NAd)(CHCMe₂Ph)(OTf)₂(DME) (Oskam, J. H.; Fox, H. H.; Yap, K. B.; McConville, D. H.; O'Dell, R.; Lichtenstein, B. J.; Schrock, R. R. J. Organomet. Chem. 1993, 459, 185), and Mo(NR)(CHCMe₂Ph)(OR_{F6})₂ ((a) Oskam, J. H.; Fox, H. H.; Yap, K. B.; McConville, D. H.; O'Dell, R.; Lichtenstein, B. J.; Schrock, R. R. J. Organomet. Chem. 1993, 459, 185. (b) Fox, H. H.; Lee, J.-K.; Park, L. Y.; Schrock, R. R. Organometallics 1993, 12, 759) were synthesized according to literature procedures. Li(2-CNPyr), Li(2-MesPyr), LiOHMT, and LiN(H)HMT were obtained by treating 2-CNPyrH, 2-MesPyrH, HMTOH, and HMTNH₂ each with one equivalent of n-BuLi at −35° C. in diethyl ether. Elemental analyses were performed by Midwest Microlab, Indianapolis, Ind.

Mo(NAd)(CHCMe₂Ph)(2-MesPyr)₂ (1a).

Mo(NAd)(CHCMe₂Ph)(OTf)₂(DME) (0.962 g, 1.26 mmol) was dissolved in ether (15 mL) and the solution was cooled to −30° C. Li(MesPyr) (0.505 g, 2.64 mmol, 2.1 equivalents) was added in portions to the ethereal suspension. The reaction mixture was allowed to warm to room temperature and was stirred for 2 h. A large amount of yellow precipitate of the product formed during the reaction. The yellow solid was filtered off and washed with cold ether. The filtrate was concentrated and recrystallized to yield another crop of product; total yield 0.70 g (75%): ¹H NMR (500 MHz, C₆D₆) δ 10.24 (s, 1H, syn Mo=CH, J_{CH}=117.5 Hz), 7.30 (d, 2H, Ar, J=7.5 Hz), 7.08-7.12 (overlapping resonances, 4H, Ar & pyr), 6.99 (t, 1H, Ar, J=7.5 Hz), 6.89 (s, 2H, Ar), 6.84 (s, 2H, Ar), 6.54 (m, 2H, Pyr), 6.23 (m, 2H, Pyr), 2.18 (s, 6H, Pyr), 2.10 (s, 12H, Pyr), 1.74 (br s, 3H, Ad), 1.44 (br s, 6H, Ad), 1.35 (s, 6H, CMe₂Ph), 1.32 (br s, 6H, Ad); ¹³C {¹H} NMR (125 MHz, C₆D₆) δ 281.8 (Mo=C), 149.9, 140.2, 139.8, 138.7, 137.6, 135.1, 133.1, 128.8, 128.6, 128.4, 128.3, 127.5, 126.3, 111.5, 110.0, 78.5, 53.3, 43.5, 35.6, 31.3, 29.9, 21.3, 21.2, 21.1. Anal. Calcd for C₄₆H₅₅N₃Mo: C, 74.07; H, 7.43; N, 5.64. Found: C, 74.09; H, 7.30; N, 5.93.

Mo(NAd)(CHCMe₂Ph)(2-CNPyr)₂ (1b).

Method 1. Mo(NAd)(CHCMe₂Ph) (OTf)₂(DME) (1.000 g, 1.31 mmol) was suspended in ether (20 mL) and the solution was cooled to −30° C. Li(2-CNPyr) (0.282, 2.87 mmol, 2.2 equivalents) was added in portions to the ethereal suspension. The reaction was warmed to room temperature and stirred for 4 h. The volatiles were then removed in vacuo. Toluene was added to the residue and the mixture was filtered through Celite in order to remove LiOTf. The filtrate was taken to dryness to yield a yellow microcrystalline solid. The crude product was recrystallized from a mixture of THF and n-pentane at −30° C. to give yellow blocks; yield 0.40 g (55%).

Method 2.

Mo(NAd)(CHCMe₂Ph)(Me₂Pyr)₂ (1.001 g, 1.77 mmol) was suspended in ether (20 mL) and the solution was cooled to −30° C. 2-CNPyrH (0.360 g, 3.91 mmol, 2.2 equivalents) was added dropwise to the ethereal suspension. The reaction was warmed to room temperature and stirred for 4 h. The volatiles were then removed in vacuo to yield a yellow microcrystalline solid. The crude product was recrystallized from a mixture of THF and n-pentane at −30° C. to give yellow blocks; yield 0.75 g (76%): ¹H NMR (500 MHz, C₆D₆) δ 14.47 (s, 1H, Mo=CH), 7.49 (s, 1H, Ar), 7.44 (s, 1H, Ar), 7.12-7.17 (overlapping resonances, 3H, Ar & Pyr), 6.99-7.04 (overlapping resonances, 3H, Ar & Pyr), 6.58 (m, 1H, Pyr), 6.45 (m, 1H, Pyr), 6.27 (m, 1H, Pyr), 1.98 (m, 6H, Ad), 1.79 (br s, 3H, Ad), 1.54 (s, 3H, CMe₂Ph), 1.35 (m, 6H, Ad), 1.08 (s, 6H, CMe₂Ph). Anal. Calcd for C₃₀H₃₃N₅Mo: C, 64.39; H, 5.94; N, 12.52. Found: C, 64.05; H, 5.95; N, 12.39.

Mo(NAd)(CHCMe₂Ph)(2-MesPyr)(OTPP) (2a).

A solution of Mo(NAd)(CHCMe₂Ph)(2-MesPyr)₂ (0.200 g, 0.270 mmol) and HOTPP (0.107 g, 0.270 mmol) in benzene (10 mL) was heated at 60° C. in a Schlenk flask for two days. The solvents were removed from the reaction mixture in vacuo. An amount of pentane sufficient to dissolve the yellow residue was added and the solution was stored at −30° C. overnight. Yellow crystals were filtered off and washed with cold pentane. Analytically pure product can be recrystallized from a mixture of toluene and pentane; yield 0.205 g (80%): ¹H NMR (500 MHz, C₆D₆) δ 11.17 (s, 1H, syn Mo=CH, J_{CH}=119.5 Hz), 7.32 (br m, 4H, Ar), 7.24 (s, 2H, Pyr), 7.19 (overlapping resonances, 4H, Ar), 6.95-7.08 (overlapping resonances, 17H, Ar), 6.84 (s, 1H, Ar), 6.52 (m, 1H, Pyr), 6.50 (m, 1H, Pyr), 6.25 (m, 1H, Pyr), 2.65 (s, 3H, Pyr), 2.26 (s, 3H, Pyr), 2.24 (s, 3H, Pyr), 1.65 (br s, 3H, Ad), 1.44 (s, 3H, CMe₂Ph), 1.29 (br s, 6H, Ad), 1.14 (s, 3H, CMe₂Ph), 1.06 (m, 6H, Ad); ¹³C NMR (125 MHz, C₆D₆) δ 285.7 (Mo=C), 159.3, 149.7, 142.4, 142.3, 140.3, 139.6, 138.9, 138.0, 137.9, 137.0, 135.3, 132.6, 132.2 (br), 130.7, 130.1, 129.3, 128.9, 128.7, 128.5, 128.1, 127.0, 126.9, 126.4, 126.0, 110.1, 109.5, 109.4, 77.0, 52.6, 42.9, 35.7, 35.6, 35.5, 32.8, 29.9, 29.8, 29.5, 21.9, 21.4. Anal. Calcd for C₆₃H₆₂N₂OMo: C, 78.89; H, 6.52; N, 2.92. Found: C, 79.09; H, 6.89; N, 2.86.

Mo(NAd)(CHCMe₂Ph)(2-MesPyr)(OBitetBr₂) (2b).

Mo(NAd)(CHCMe₂Ph)(2-MesPyr)₂ (0.192 g, 0.250 mmol) was suspended in ether (10 mL) and cooled to −30° C. for 2 h. Br₂BitetOH (0.156 g, 0.280 mmol, 1.10 equivalents) was then added to the ethereal suspension. The reaction was warmed to room temperature and stirred for two days. An amount of pentane sufficient to dissolve the yellow residue was added and the solution was stored at −30° C. overnight. The yellow crystals were collected via filtration and washed with cold pentane. Analytically pure product (a mixture of diastereomers in ˜1:1 ratio) can be obtained through recrystallization from pentane; yield 0.153 g (53%): ¹H NMR (500 MHz, C₆D₆, selected resonances for R-diastereomer) δ 12.47 (s, 1H, syn Mo=CH, J_{CH}=120.5 Hz), 1.00 (s, 9H, OSi^{t}Bu), 0.32 (s, 3H, OSiMe₂), −0.18 (s, 3H, OSiMe₂): ¹H NMR (500 MHz, C₆D₆; selected resonances for S-diastereomer) δ 13.14 (s, 1H, Mo=CH, J_{CH}=120.0 Hz), 1.01 (s, 9H, OSi^{t}Bu), 0.32 (s, 3H, OSiMe₂), −0.50 (s, 3H, OSiMe₂); ¹³C NMR of both diastereomers (125 MHz, C₆D₆) δ 290.3, 288.5 (Mo=C), 158.5, 157.8, 149.6, 149.3, 147.9, 147.5, 140.2, 140.0, 139.7, 139.4, 139.2, 138.7, 137.1, 136.9, 136.8, 136.6, 136.5, 136.2, 136.1, 133.7, 132.0, 131.7, 131.5, 131.2, 130.8, 130.6, 128.9, 127.5, 127.3, 126.3, 126.2, 114.0, 113.7, 112.6, 112.0, 111.2, 109.5, 109.0, 77.9, 77.5, 53.5, 52.9, 43.2, 35.7, 32.4, 32.1, 31.3, 30.9, 30.7, 30.1, 30.0, 29.6, 29.4, 27.5, 26.6, 25.9, 23.4, 23.2, 23.0, 22.7, 22.1, 21.9, 21.6, 21.4, 21.3, 19.1, 18.9, −2.5, −2.8, −3.15. Anal. Calcd for C₅₉H₄N₂Br₂O₂SiMo: C, 62.87; H, 6.62; N, 2.49. Found: C, 62.59; H, 6.52; N, 2.43.

Mo(NAd)(CHCMe₂Ph)(2-MesPyr)(OHIPT) (2c).

Solid sodium 2-Mespyrrolide (80.4 mg, 0.388 mmol, 1 equiv) was added to a solution of Mo(NAd)(CHCMe₂Ph)(OTf)(OHIPT) (398 mg, 0.388 mmol, 1 equiv) in benzene (20 mL). The reaction mixture was heated at 80° C. for 10 hours, cooled to 22° C., and filtered through Celite. The volatiles were removed under vacuum. The yellow product was recrystallized from a mixture of pentane and tetramethylsilane; yield 183 mg (45%): ¹H NMR (500 MHz, C₆D₆) δ 12.25 (s, 1H, syn-Mo=CH, J_{CH}=120.0 Hz), 7.23 (s, 2H, Ar), 7.20 (s, 2H, Ar), 7.18-7.10 (m, 4H, Ar), 7.06 (d, 1H, Ar, J=7.5 Hz), 7.03 (d, 2H, Ar, J=7.5 Hz), 6.83 (s, 1H, Ar), 6.80 (t, 1H, Ar, J=7.5 Hz), 6.76 (s, 1H, Ar), 6.36 (t, 1H, NC₄H₃, J=2.5 Hz), 6.15 (dd, 1H, NC₄H₃, J=2.5 Hz, 1.2 Hz), 5.99 (dd, 1H, NC₄H₃, J=2.5 Hz, 1.2 Hz), 3.05 (sept, 2H, MeCHMe, J=7.0 Hz), 2.97 (sept, 4H, MeCHMe, J=7.0 Hz), 2.33 (s, 3H, Me), 2.16 (s, 3H, Me), 2.15 (s, 3H, Me), 1.73 (br, 3H), 1.53 (s, 3H, Me), 1.48 (s, 3H, Me), 1.36 (app q, 12H, MeCHMe), 1.33 (br, 9H), 1.27 (d, 6H, MeCHMe, J=7.0 Hz), 1.23 (d, 6H, MeCHMe, J=7.0 Hz), 1.15 (t, 12H, MeCHMe, J=7.0 Hz), 1.11 (br, 3H); ¹³C NMR (125 MHz, C₆D₆) δ 288.0, 160.2, 150.6, 148.2, 147.7, 147.6, 140.0, 139.6, 139.3, 136.9, 136.7, 135.8, 135.2 (br), 133.3, 131.3, 129.1 (br), 128.9 (br), 127.5, 126.4, 122.1, 121.6, 120.8, 111.5, 111.4, 111.2, 111.1, 100.5, 77.1, 53.2, 43.7, 35.9, 35.1, 35.0, 34.6, 31.8, 31.6, 30.2, 30.1, 26.4, 26.1, 24.7, 24.7, 24.6. Anal. Calcd for C₆₉H₉₀MoN₂O: C, 78.22; H, 8.56; N, 2.64. Found: C, 77.91; H, 8.60; N, 2.65.

Mo(NAd)(CHCMe₂Ph)(OTf)(OHIPT) (3).

Solid LiOHIPT (371 mg, 0.735 mmol) was added to a suspension of Mo(NAd)(CHCMe₂Ph)(OTf)₂(dme) (563 mg, 0.735 mmol) in benzene (20 mL). The reaction mixture was heated at 80° C. for 24 hours, cooled to 22° C., and filtered through Celite. The solvents were removed in vacuo to yield a yellow-brown solid; yield 750 mg (99%): ¹H NMR (500 MHz, C₆D₆) δ 12.35 (s, 1H, syn-Mo=CH, J_{CH}=123.0 Hz), 7.33 (s, 4H, Ar), 7.25-7.00 (m, 7H, Ar), 6.84 (t, 1H, Ar, J=7.5 Hz), 2.98 (sept, 4H, MeCHMe, J=7.0 Hz), 2.92 (sept, 2H, MeCHMe, J=7.0 Hz), 1.78 (br, 6H), 1.74 (br, 6H), 1.58 (s, 3H, Me), 1.37 (d, 6H, MeCHMe, J=7.0 Hz), 1.35 (br, 6H), 1.33 (t, 12H, MeCHMe, J=7.0 Hz), 1.17 (d, 6H, MeCHMe, J=7.0 Hz), 1.11 (t, 12H, MeCHMe, J=7.0 Hz); ¹³C NMR (125 MHz, C₆D₆) δ 301.5, 159.8, 152.0, 149.7, 149.5, 149.2, 148.5, 147.8, 147.2, 134.1, 132.3, 131.8, 128.9, 128.7, 127.3, 126.9, 123.3, 122.2, 122.0, 121.7, 81.5, 54.0, 44.0, 35.9, 34.8, 34.5, 31.6 (br), 30.2 (br), 25.4 (br), 24.7 (br); ¹⁹F NMR (282 MHz, C₆D₆) δ −75.4. Anal. Calcd for C₅₇H₇₆F₃MoNO₄S: C, 66.84; H, 7.48; N, 1.37. Found: 66.75; H, 7.50; N, 1.44.

Mo(NAd)(CHCMe₂Ph)(OTf)(OHIPT)(PMe₃) (3(PMe₃)).

Trimethylphosphine (57 mL, 0.552 mmol, 1.50 equiv) was added to a solution of Mo(NAd)(CHCMe₂Ph)(OTf)(OHIPT) (378 mg, 0.368 mmol) in benzene (20 mL). The reaction mixture was stirred at room temperature for 15 minutes and the volatiles were removed in vacuo. Pentane was added and the off-white solid was collected on a medium porosity frit; yield 303 mg (75%): ¹H NMR (500 MHz, C₆D₆) δ 12.74 (d, 1H, syn-Mo=CH, J_{CH}=122.2 Hz, J_{PH}=4.6 Hz), 7.48 (s, 1H, Ar), 7.32 (d, 2H, Ar, J=11.6 Hz), 7.25-7.19 (m, 3H, Ar), 7.10 (t, 2H, Ar, J=7.5 Hz), 7.04 (s, 1H, Ar), 6.99 (t, 1H, Ar, J=7.5 Hz), 6.87 (d, 2H, Ar, J=7.5 Hz), 6.80 (t, 1H, Ar, J=7.5 Hz), 3.73 (sept, 1H, MeCHMe, J=6.5 Hz), 3.58 (sept, 1H, MeCHMe, J=6.5 Hz), 3.08 (sept, 1H, MeCHMe, J=6.5 Hz), 2.95 (sept, 1H, MeCHMe, J=6.5 Hz), 2.83 (sept, 1H, MeCHMe, J=6.5 Hz), 2.76 (sept, 1H, MeCHMe, J=6.5 Hz), 2.20 (br, 6H), 2.03 (s, 3H, Me), 1.93 (br, 3H), 1.80 (d, 3H, MeCHMe, J=6.5 Hz), 1.73 (d, 3H, MeCHMe, J=6.5 Hz), 1.58-1.14 (m, 33H), 1.05 (d, 3H, MeCHMe, J=6.5 Hz), 1.02 (d, 3H, MeCHMe, J=6.5 Hz), 0.39 (d, 9H, PMe₃, J_{PH}=10.2 Hz); ¹³C NMR (125 MHz, C₆D₆:CD₂Cl₂=1:1) δd 314.5 (d, J_{CP}=19.9 Hz), 161.5, 149.2 (br), 148.1, 147.9 (br), 138.4 (br), 137.5 (br), 134.2 (br), 133.3 (br), 130.1, 129.0 (br), 127.1 (br), 126.6, 126.5, 122.9 (br), 121.5 (br), 120.8 (br), 117.9 (br), 75.2, 52.0, 44.8, 36.3, 34.9 (br), 31.1 (br), 30.3 (br), 25.0 (br), 16.1 (t, J_{CP}=31.0 Hz); ¹⁹F NMR (282 MHz, C₆D₆:CD₂Cl₂=1:1) δ −75.2; ³¹P NMR (121 MHz, C₆D₆:CD₂Cl₂=1:1) δ 8.6. Anal. Calcd for C₆₀H₈₅F₃MoNO₄PS: C, 65.49; H, 7.79; N, 1.27. Found: 65.56; H, 7.78; N, 1.13.

Mo(NAd)(CHCMe₂Ph)(2-CNPyr)(OHIPT) (4).

Mo(NAd)(CHCMe₂Ph)(OTf) (OHIPT) (0.300 g, 0.29 mmol) was dissolved in benzene (15 mL) and Li(2-CNPyr) (0.029 g, 0.30 mmol, 1.1 equivalents) was added in portions to the solution. The reaction mixture was stirred at room temperature for 18 h. The white LiOTf salt was removed by filtration through Celite, and the solvents were removed from the filtrate in vacuo. Tetramethylsilane was added to the orange residue to produce some dark orange solids. The orange solids were washed with n-pentane and the washings were combined and taken to dryness in vacuo. Upon addition of fresh n-pentane, a yellow microcrystalline solid was obtained. The yellow solid was recrystallized from a concentrated solution of n-pentane; yield 70 mg (25%): ¹H NMR (500 MHz, C₆D₆) g 12.69 (s, 1H, syn Mo=CH, J_{CH}=121.0 Hz), 7.11-7.24 (overlapping resonances, 8H, Ar), 7.01-7.04 (m, 3H, Ar), 6.84 (t, 1H, Ar, J=7.5 Hz), 6.78 (m, 1H, Pyr), 6.21 (m, 1H, Pr), 6.09 (m, 1H, Pyr), 3.00 (overlapping sept, 4H, ⁱPr), 2.93 (sept, 2H, CHMe₂, J=7.0 Hz), 1.80 (m, 6H, Ad), 1.66 (m, 3H, Ad), 1.59 (s, 3H, CMe₂Ph), 1.51 (s, 3H, CMe₂Ph), 1.45 (m, 3H, Ad), 1.31-1.37 (overlapping resonances, 15H, Ad & CHMe₂), 1.25 (d, 9H, CHMe₂, J=7.0 Hz), 1.13-1.19 (overlapping resonances, 15H, CHMe₂); ¹³C NMR (125 MHz, C₆D₆) δ 296.2 (Mo=C), 159.5, 149.8, 148.1, 147.4, 147.0, 142.2, 134.5, 131.8, 131.6, 127.4, 126.3, 122.1, 121.9, 121.6, 118.4, 113.2, 111.1, 78.7, 53.9, 43.4, 35.7, 34.6, 34.2, 32.8, 32.0, 31.5, 30.8, 30.0, 29.9, 25.1, 24.9, 24.8, 24.3, 24.2. Anal. Calcd for C₆₁H₇₉N₃OMo: C, 75.82; H, 8.24; N, 4.35. Found: C, 75.90; H, 8.16; N, 4.32.

Mo(NAd)(CHCMe₂Ph)(OCMe₃)(OHIPT) (5).

LiOCMe₃ (80.0 mg, 0.360 mmol, 1 equiv) was added to a solution of Mo(NAd)(CHCMe₂Ph)(OTf)(OHIPT) (368.4 mg, 0.360 mmol, 1 equiv) in benzene (20 mL). The reaction mixture was stirred at room temperature for 24 hours and the reaction mixture was filtered through Celite. The volatiles were removed from the filtrate in vacuo. The residue was recrystallized from a mixture of pentane and tetramethylsilane to yield a yellow solid; yield 75 mg (22%): ¹H NMR (500 MHz, C₆D₆) δ 11.16 (s, 1H, syn-Mo=CH, J_{CH}=119.0 Hz), 7.26 (s, 4H, Ar), 7.21 (d, 2H, Ar, J=1.5 Hz), 7.11 (d, 2H, Ar, J=7.5 Hz), 7.08 (d, 2H, Ar, J=7.5 Hz), 7.02 (t, 1H, Ar, J=7.5 Hz), 6.82 (t, 1H, Ar, J=7.5 Hz), 3.16 (sept, 4H, MeCHMe, J=6.5 Hz), 2.95 (sept, 2H, MeCHMe, J=6.5 Hz), 1.90 (br, 6H), 1.71 (app q, 6H, J=11.8 Hz), 1.55 (s, 3H, Me), 1.45 (br, 6H), 1.41 (d, 6H, MeCHMe, J=6.5 Hz), 1.35 (t, 12H, MeCHMe, J=6.5 Hz), 1.28 (d, 6H, MeCHMe, J=6.5 Hz), 1.23 (t, 12H, MeCHMe, J=6.5 Hz), 1.01 (s, 9H, OCMe₃); ¹³C NMR (125 MHz, C₆D₆) δ 263.1, 161.8, 150.9, 148.2, 148.1, 147.7, 135.8, 132.7, 131.6, 128.9, 128.7, 128.3, 127.1, 126.3, 121.6, 121.5, 120.2, 77.9, 74.1, 49.7, 45.0, 36.5, 34.8, 34.7, 32.0, 31.5, 30.5, 30.2, 26.5, 26.3, 26.1, 25.9, 25.4, 25.2, 25.1, 24.9, 24.8, 24.7, 24.4. Anal. Calcd for C₆₀H₈₅MoNO₂: C, 75.99; H, 9.03; N, 1.48. Found: C, 75.64; H, 8.80; N, 1.55.

Mo(NAr)(CHCMe₂Ph)(OR_{F6})(OHMT) (6a).

Mo(NAr)(CHCMe₂Ph)(OR_{F6})₂ (0.3118 g, 0.41 mmol) was dissolved in Et₂O and LiOHMT (0.1370 g, 0.41 mmol) was added in one portion. The reaction mixture was left stirring at RT overnight. The volatiles were removed and the crude was dissolved in minimal pentane and placed at −35° C. overnight to yield orange crystalline solid (0.1600 g, 43%): ¹H NMR (400 MHz, C₆D₆) δ 11.59 (s, 1H, Mo=CHCMe₂Ph, J_{CH}=124.2 Hz), 7.25-6.85 (overlapping peaks, 15H, aromatics), 3.45 (sept, 2H, CHMe₂), 2.30 (s, 12H, HMTO), 2.20 (s, 6H, HMTO), 1.79 (s, 3H, CH₃), 1.31 (d, 6H, CHMe₂), 1.25 (s, 3H, CH₃), 1.18 (d, 6H, CHMe₂), 0.78 (s, 3H, CH₃); ¹³C NMR (100 MHz, C₆D₆) δ 281.3 (Mo=CHCMe₂Ph), 158.4, 153.4, 148.7, 143.0, 136.6, 132.3, 130.0, 129.2, 128.4, 128.4, 126.4, 125.6, 123.3, 123.1, 122.9, 54.3, 31.6, 29.8, 28.3, 24.3, 23.8, 20.9, 20.8, 19.2; ¹⁹F NMR (376 MHz, C₆D₆) δ −77.2 (q, 3F, CF₃), −77.7 (q, 3F, CF₃). Anal. Calcd for C₅₀H₅₇F₆MoNO₂: C, 65.71; H, 6.29, N, 1.53. Found: C, 65.65; H, 6.11; N, 1.34.

Mo(NAr′)(CHCMe₂Ph)(OR_{F6})(OHMT) (6b).

The procedure is the same as that of 6a, employing Mo(NAr′)(CHCMe₂Ph)(OR_{F6})₂ (0.3065 g, 0.43 mmol) and LiOHMT (0.1453 g, 0.43 mmol) to yield yellow solid (0.2977 g, 80%): ¹H NMR (400 MHz, C₆D₆) δ 11.40 (s, 1H, Mo=CHCMe₂Ph, J_{CH}=123.8 Hz), 7.05-6.55 (overlapping peaks, 15H, aromatics), 2.19 (s, 6H, HMTO), 2.15 (s, 6H, HMTO), 2.06 (s, 6H, Ar′ CH₃), 2.03 (s, 6H, HMTO), 1.45 (s, 3H, CH₃), 1.22 (s, 3H, CH₃), 0.80 (s, 3H, CH₃); ¹³C NMR (100 MHz, C₆D₆) δ 281.7 (Mo=CHCMe₂Ph), 158.0, 156.0, 148.6, 136.6, 136.4, 136.2, 135.7, 135.3, 132.0, 129.6, 129.1, 128.4, 128.1, 128.0, 126.9, 126.5, 126.2, 126.0, 125.9, 122.6, 53.6, 40.3, 31.1, 29.6, 29.1, 20.8, 20.6, 19.5, 19.2; ¹⁹F NMR (376 MHz, C₆D₆) δ −77.4 (q, 3F, CF₃), −77.6 (q, 3F, CF₃). Anal. Calcd for C₄₆H₄₄F₆MoNO₂: C, 64.41; H, 5.76, N, 1.63. Found: C, 64.81; H, 5.82; N, 1.38.

Mo(NAr^{iPr})(CHCMe₂Ph)(ORᵣ₆)(OHMT) (6c).

The procedure is the same as that of 6a, employing Mo(NAr^{iPr})(CHCMe₂Ph)(OR_{F6})₂ (0.3602 g, 0.50 mmol) and LiOHMT (0.1675 g, 0.50 mmol) to yield orange crystalline solid (0.3672 g, 71%): ¹H NMR (400 MHz, C₆D₆) δ 11.20 (s, 1H, Mo=CHCMe₂Ph, J_{CH}=124.5 Hz), 7.14 (dd, 2H, aromatic), 7.08 (td, 2H, Ar^{iPr}), 7.02-6.78 (overlapping peaks, 8H, aromatics), 6.76 (s, 2H, HMTO), 6.67 (s, 2H, HMTO), 3.31 (sept, 1H, CHMe₂), 2.14 (s, 6H, HMTO), 2.08 (s, 6H, HMTO), 2.04 (s, 6H, HMTO), 1.49 (s, 3H, CH₃), 1.43 (s, 3H, CH₃), 1.15 (d, 3H, CH(Me)CH₃), 1.13 (d, 3H, CH(CH₃)Me), 0.94 (s, 3H, CH₃); ¹³C NMR (100 MHz, C₆D₆) δ 281.7 (Mo=CHCMe₂Ph), 157.9, 155.3, 149.0, 146.7, 136.5, 136.4, 136.0, 135.4, 132.1, 129.7, 128.7, 128.4, 127.9, 127.9, 126.2, 125.5, 122.9, 54.3, 32.4, 30.5, 28.2, 23.6, 21.1, 20.9, 20.5, 18.6; ¹⁹F NMR (375 MHz, C₆D₆) δ −77.7 (q, 3F, CF₃), −78.1 (1, 3F, CF₃). Anal. Calcd for C₄₇H₅₁F₆MoNO₂: C, 64.75; H, 5.90, N, 1.61. Found: C, 64.60; H, 6.01; N, 1.41.

Mo(NAd)(CHCMe₂Ph)(OR_{F6})(OHMT) (6d).

The procedure is the same as that of 6a, employing Mo(NAd)(CHCMe₂Ph)(OR_{F6})₂ (0.1900 g, 0.26 mmol) and LiOHMT (0.0864 g, 0.26 mmol) to yield yellow crystalline solid (0.1353 g, 59%): ¹H NMR (400 MHz, C₆D₆) δ 10.64 (s, 1H, Mo=CHCMe₂Ph, J_{CH}=122.7 Hz), 7.25-7.08 (overlapping peaks, 4H, aromatics), 7.05-6.93 (overlapping peaks 3H, aromatics), 6.92-6.53 (overlapping peaks 3H, aromatics), 6.72 (s, 2H, HMTO), 2.26 (s, 6H, HMTO), 2.20 (s, 6H, HMTO), 1.97 (s, 6H, HMTO), 1.80 (s, 3H, Ad), 1.70-1.50 (overlapping peaks, 9H, MoCHCMe₂Ph+Ad), 1.47-1.30 (overlapping peaks, 9H, MoCHCMe₂Ph+Ad), 1.16 (s, 3H, CH₃); ¹³C NMR (100 MHz, C₆D₆) δ 276.0 (Mo=CHCMe₂Ph), 158.7, 150.3, 137.5, 136.6, 136.4, 136.3, 131.9, 130.5, 129.2, 128.7, 126.7, 126.1, 122.2, 76.9, 50.5, 43.7, 35.6, 33.2, 31.0, 29.9, 21.1, 20.7, 20.6; ¹⁹F NMR (376 MHz, C₆D₆) δ −77.4 (q, 3F, CF₃), −78.1 (q, 3F, CF₃). Anal. Calcd for C₄₈H₅₅F₆MoNO₂: C, 64.93; H, 6.24, N, 1.58. Found: C, 64.95; H, 6.12; N, 1.56.

Mo(NAr′)(CHCMe₂Ph)(OR_{F6})(N(H)HMT) (7a).

The procedure is the same as that of 6a, employing Mo(NAr′)(CHCMe₂Ph)(OR_{F6})₂ (0.2094 g, 0.30 mmol) and LiN(H)HMT (0.0990 g, 0.30 mmol) to yield yellow solid (0.1205 g, 48%): ¹H NMR (400 MHz, C₆D₆) δ 11.86 (s, 1H, Mo=CHCMe₂Ph, J_{CH}=121.2 Hz), 7.82 (s, 1H, MoN(H)HMT), 7.25-7.10 (overlapping peaks, 3H, aromatics), 7.00-6.55 (overlapping peaks, 11H, aromatics), 6.40 (s br, 1H, aromatic), 2.38 (s, 3H, CH₃), 2.23 (s, 6H, CH₃), 2.19 (s, 3H, CH₃), 2.10 (s, 3H, CH₃), 2.03 (s, 6H, CH₃), 1.67 (s, 3H, CH₃), 1.19 (s, 3H, CH₃), 1.16 (s, 3H, CH₃), 1.00 (s, 3H, CH₃); ¹³C NMR (100 MHz, C₆D₆) δ 287.3 (Mo=CHCMe₂Ph), 156.1, 149.5, 148.2, 135.6, 131.3, 130.7, 130.2, 129.2, 129.2, 128.9, 128.6, 127.1, 126.1, 125.8, 121.1, 54.5, 30.5, 28.8, 21.2, 20.6, 20.5, 20.3, 20.0, 19.4, 19.3; ¹⁹F NMR (376 MHz, C₆D₆) δ −76.7 (q, 3F, CF₃), −77.2 (q, 3F, CH₃). Anal. Calcd for C₄₆H₅₀F₆MoN₂O: C, 64.48; H, 5.88, N, 3.27. Found: C, 64.20; H, 6.01; N, 3.22.

Mo(NAr^{iPr})(CHCMe₂Ph)(OR_{F6})(N(H)HMT) (7b). The procedure is the same as that of 6a, employing Mo(NAr^{iPr})(CHCMe₂Ph)(OR_{F6})₂ (0.2959 g, 0.41 mmol) and LiN(H)HMT (0.1372 g, 0.41 mmol) to yield orange crystalline solid (0.3260 g, 92%): ¹H NMR (400 MHz, C₆D₆) δ; 11.72 (s, 1H, Mo=CHCMe₂Ph, J_{CH}=117.9 Hz), 7.99 (s, 1H, MoN(H)HMT), 7.23 (dd, 1H, aromatic), 7.08 (dd, 1H, aromatic), 7.05-6.70 (overlapping peaks, 12H, aromatics), 6.72 (s br, 2H, aromatic), 3.22 (sept, 1H, CHMe₂), 2.31 (s, 6H, CH₃), 2.24 (s, 6H, CH₃), 1.94 (s br, 6H, CH₃), 1.53 (s, 3H, CH₃), 1.25 (d, 3H, CH(Me)CH₃), 1.19 (s, 3H, CH₃), 1.12 (d, 3H, CH(CH₃)Me), 0.82 (s, 3H, CH₃); ¹³C NMR (100 MHz, C₆D₆) δ 286.5 (Mo=CHCMe₂Ph), 155.5, 149.4, 148.0, 146.1, 137.9, 137.4, 137.4, 135.5, 130.3, 129.0, 128.5, 128.2, 126.3, 125.7, 125.6, 125.5, 121.5, 55.6, 30.4, 30.1, 27.8, 24.4, 22.6, 21.2, 20.5, 19.4; ¹⁹F NMR (376 MHz, C₆D₆) δ −77.1 (q, 3F, CF₃), −77.7 (q, 3F, CH₃). Anal. Calcd for C₄₇H₅₂F₆MoN₂O: C, 64.82; H, 6.02, N, 3.22. Found: C, 64.43; H, 5.89; N, 3.15.

Mo(NAd)(CHCMe₂Ph)(OR_{F6})(HMT) (8).

Mo(NAd)(CHCMe₂Ph)(OR_{F6})₂ (DME) (0.300 g, 0.360 mmoles) and LiHMT (0.1159 g, 0.36 mmoles) were added to 3 mL of toluene at RT and the mixture was stirred for 12 h. The solution was placed at −35° C. overnight and then passed through celite. The celite was washed with cold toluene until the color was gone. At this point the solvent was removed from the filtrate under reduced pressure and the product was washed with pentane 3 times and twice with TMS to induce precipitation of the product, which was collected by filtration (0.3080 g, 95%). Orange-yellow crystals were grown by dissolving 8 in pentane and placing the solution at −35° C. a few days: ¹H NMR (400 MHz, C₆D₆) δ 10.99 (s, 1H, MoCH, J_{CH}=120.2 Hz), 7.40 (d, 2H, HMT), 7.20-7.10 (m, 3H, Ph), 7.03 (t, 1H, HMT), 6.90 (s, 2H, Mes), 6.84 (m, 2H, Ph), 6.79 (s, 2H, Mes), 2.23 (s, 6H, o-Mes), 2.21 (s, 6H, o-Mes), 2.16 (s, 6H, p-Mes), 1.71 (s, 3H, Ad), 1.44 (s, 6H, CMe₂Ph), 1.36 (s, 6H, CMe₂Ph syn), 1.33 (s, 6H, Ad), 1.13 (s, 3H, (CF₃)₂CH₃C); ¹³C NMR (100 MHz, C₆D₆) δ 279.4 (MoCH, J_{CH}=120.23 Hz), 173.7 (CF₃), 150.9, 150.5, 145.6, 136.7, 136.2, 136.0, 135.7, 129.7, 129.5, 129.3, 128.1, 125.9, 77.6, 53.5, 42.5, 35.6, 35.2, 30.4, 30.0, 22.1, 21.8, 21.1; ¹⁹F NMR (375 MHz, C₆D₆) δ −77.2 (q, 1F), −77.9 (q, 1F). Anal. Calcd for C₄₈H₅₅F₆MoNO: C, 66.12; H, 6.36, N, 1.61. Found: C, 66.37; H, 6.30; N, 1.74.

Mo(NAd)(CHCMe₂Ph)(OR_{F9})₂ Mo(NAd)(CHCMe₂Ph)(OTf)₂DME (0.

503 g, 0.658 mmol) was suspended in Et₂O and stored at −35° C. for 1 h. Then, LiOR_{F9} (0.319 g, 1.32 mmol) was added in one portion and the mixture was allowed to warm-up to RT and stir for 12 h. The solvent was removed under vacuo; the crude solid redissolved in CH₂Cl₂ and passed through Celite to remove LiOTf. Solvent was removed from the filtrate under reduced pressure and the crude was triturated with pentane to precipitate a yellow solid, which was collected by filtration (0.370 g, 66%): ¹H NMR (400 MHz, C₆D₆) δ 13.65 (s, 0.35H, MoCH, J_{CH}=151.9 Hz), 12.61 (s, 1H, MoCH, J_{CH}=121.9 Hz), 7.30 (d, 1H, Ar syn), 7.24-7.14 (m, 4H, Ar anti+syn), 7.06-6.98 (m, 1.5H, Ar anti+syn), 3.20-2.90 (overlapping peaks, 3.5H, DME anti), 2.05 (s, 2H, CMe₂Ph anti), 1.82 (m, 8H, Ad anti+syn), 1.73 (s br, 4H, Ad anti+syn), 1.47 (s, 6H, CMe₂Ph syn), 1.28 (m, 8H, Ad anti+syn); ¹³C NMR (100 MHz, C₆D₆) δ 307.7 (MoCH, J_{CH}=151.9 Hz), 292.6 (MoCH, J_{CH}=121.9 Hz), 149.9 (CF₃ anti), 149.0 (CF₃ syn), 128.8, 128.7, 127.1, 126.8, 126.7, 125.7, 122.9, 120.0, 84.0 (C(CF₃)₃ anti), 81.8 (C(CF₃)₃ syn), 53.7 (CMe₂Ph anti), 50.6 (CMe₂Ph syn), 43.8, 43.1, 35.4, 31.1, 29.8, 29.6, 26.7; ¹⁹F NMR (376 MHz, C₆D₆) δ −74.5 (s, 1F, syn), −74.7 (s, 0.35F, anti). Anal. Calcd for C₁₁₆H₁₁₈F₇₂Mo₄N₄O₁₀: C, 40.04; H, 3.42, N, 1.61. Found: C, 40.00; H, 3.50; N, 1.50.

In Situ Reactions.

All NMR reactions were carried out in 0.70 mL of C₆D₆ in Teflon-sealed J-young tubes unless otherwise noted with the specified amounts of reagents and at the specified temperature as stated below.

Mo(NAr^{m})(CHCMe₂Ph)(OR_{F6})(HMT)

Mo(NAr^{m})(CHCMe₂Ph)(OR_{F6})₂(DME) (0.258 g, 0.323 mmol) and LiHMT (0.103 g, 0.321 mmol) were mixed in benzene (2.00 mL) at RT in a vial, capped and left stirring at 12 h to get 100% of product: ¹H NMR (400 MHz, C₆D₆) δ 10.90 (s, 1H, Mo=CHCMe₂Ph, J_{CH}=122.0 Hz), 7.34 (dd, 2H, aromatic), 7.24 (td, 1H, aromatic), 7.15-7.02 (overlapping peaks, 3H, aromatic), 6.98 (dd, 2H, aromatic), 6.86 (s, 4H, HMT), 6.74 (s, 1H, Ar^{m}), 6.68 (s, 1H, Ar^{m}), 6.61 (s, 1H, Ar^{m}), 2.21 (s, 6H, CH₃), 2.18 (s, 3H, CH₃), 2.11 (s, 3H, CH₃), 2.06 (s, 12H, CH₃), 2.03 (s, 3H, CH₃), 1.32 (s, 3H, CH₃); ¹³C NMR (100 MHz, C₆D₆) δ 282.8 (MoCHCMe₂Ph); ¹⁹F NMR (376 MHz, C₆D₆) δ −77.4 (q, 3F, CF₃), −78.6 (q, 3F, CF₃).

Mo(NAr′)(CHCMe₂Ph)(OR_{F6})(HMT)

Mo(NAr′)(CHCMe₂Ph)(OR_{F6})₂ (0.010 g, 0.014 mmol) and LiHMT (0.005 g, 0.014 mmol) were mixed together and heated at 80° C. for 5 d to get 90% of products: ¹H NMR (400 MHz, C₆D₆) δ 10.93 (s, 1H, Mo=CHCMe₂Ph, 90%).

Mo(NAr)(CHCMe₂Ph)(OR_{F6})(HMT)

Mo(NAr)(CHCMe₂Ph)(OR_{F6})₂ (0.010 g, 0.013 mmol) and LiHMT (0.004 g, 0.013 mmol) were mixed and heated at 80° C. for 5 d to get 18% of products: ¹H NMR (400 MHz, C₆D₆) δ 10.90 (s, 1H, Mo=CHCMe₂Ph).

Mo(NAd)(CHCMe₂Ph)(OR_{F6})(TIPT)

Mo(NAd)(CHCMe₂Ph)(OR_{F6})₂DME (0.300 g, 0.362 mmol) and LiTIPT (0.146 g, 361 mmol) were mixed together and heated at 80° C. for 5 d to get full conversion to a single product: ¹H NMR (400 MHz, C₆D₆) δ 11.75 (s, 1H, Mo=CHCMe₂Ph).

Mo(NAr′)(CHCMe₂Ph)(OR_{F6})(TIPT)

Mo(NAr′)(CHCMe₂Ph)(OR_{F6})₂ (0.010 g, 0.014 mmol) and LiTIPT (0.006 g, 0.014 mmol) were mixed together and heated at 80° C. for 2 wk to get 28% of products: ¹H NMR (400 MHz, C₆D₆) δ 12.14 (s, 1H, Mo=CHCMe₂Ph), 11.26 (s, 0.6H, Mo=CHCMe₂Ph), 10.95 (s, 1H, Mo=CHCMe₂Ph).

Mo(NAr)(CHCMe₂Ph)(OR_{F6})(TIPT)

Mo(NAr)(CHCMe₂Ph)(OR_{F6})₂ (0.010 g, 0.013 mmol) and LiTIPT (0.005 g, 0.013 mmol) were mixed and heated at 80° C. for 5 d to get 7% of products: ¹H NMR (400 MHz, C₆D₆) δ 12.24 (s, 1H, Mo=CHCMe₂Ph), 11.35 (s, 0.75H, Mo=CHCMe₂Ph).

Mo(NAd)(CHCMe₂Ph)(OR_{F9})(HMT)

Mo(NAd)(CHCMe₂Ph)(OR_{F9})₂ (0.300 g, 0.354 mmol) and LiHMT (0.113 g, 0.354 mmol) were mixed together in toluene (3.00 mL) at RT for 12 h to get a single product: ¹H NMR (400 MHz, C₆D₆) δ 11.24 (s, 1H, Mo=CHCMe₂Ph, J_{CH}=119.2 Hz), 7.40-6.70 (overlapping peaks, 12H, aromatics), 2.22 (s, 6H, CH₃), 2.21 (s, 3H, CH₃), 2.19 (s, 6H, CH₃), 2.07 (s, 6H, CH₃), 1.68 (s, 3H, Ad), 1.50-1.40 (overlapping peaks, 6H, Ad+CH₃), 1.30 (s, 6H, Ad), 0.96 (s, 3H, Ad); ¹³C NMR (100 MHz, C₆D₆) δ 285.3 (MoCHCMe₂Ph), 174.4, 150.8, 150.4, 145.7, 141.9, 139.4, 136.5, 136.4, 135.7, 130.7, 130.3, 129.6, 129.4, 128.5, 128.3, 127.7, 126.5, 126.0, 79.2 (OC(CF₃)₃), 54.4, 41.9, 35.4, 29.9, 22.0, 21.7, 21.1, 21.1, 20.0; ¹⁹F NMR (376 MHz, C₆D₆) δ −73.4 (s, 9F, CF₃).

X-Ray Structure Determination:

Low-temperature diffraction data (φ- and ω-scans) were collected on a Siemens Platform three-circle diffractometer coupled to a Bruker-AXS Smart Apex CCD detector with graphite-monochromated Mo Kα radiation (λ=0.71073 Å) for 2b and 3(PMe₃), on a Bruker D8 three-circle diffractometer coupled to a Bruker-AXS Smart Apex CCD detector with graphite-monochromated Cu Kα radiation (2=1.54178 A) for 1b, and on a Bruker-AXS X8 Kappa Duo diffractometer coupled to a Smart Apex2 CCD detector with Mo Kₐ radiation (λ=0.71073 Å) from an IμS micro-source for the structure of compounds 2a, 2c, 4, 5, 6d, 7b, and 8. All structures were solved by direct methods using SHELXS (Sheldrick, G. M. Acta Cryst. 1990, A46, 467-473) and refined against F² on all data by full-matrix least squares with SHELXL-97 (Sheldrick, G. M. Acta Cryst. 2008, A64, 112-122) using established refinement techniques (Müller, P. Crystallography Reviews 2009, 15, 57-83). All non-hydrogen atoms were refined anisotropically. Unless otherwise noted below all hydrogen atoms were included into the model at geometrically calculated positions and refined using a riding model. The isotropic displacement parameters of all hydrogen atoms were fixed to 1.2 times the U value of the atoms they are linked to (1.5 times for methyl groups). All disordered atoms were refined with the help of similarity restraints on the 1,2- and 1,3-distances and displacement parameters as well as rigid bond restraints for anisotropic displacement parameters.

Compound 1b:

Compound 1b crystallizes in the tetragonal space group P4/nnc with four octameric molecules of 3d (monomeric subunit corresponds to Mo(NAd)(CHCMe₂Ph)(2-CNPyr)₂) in the unit cell, corresponding to two Mo centers per asymmetric unit. In addition the asymmetric unit contains highly disordered solvent, which was modeled to be a mixture of thf and pentane. This solvent disorder is uncommonly complex and all solvent molecules are found near crystallographic fourfold axes. The first solvent site consists of eight-fold disordered thf (two independent) components, the second site of eight-fold disordered pentane (two independent positions) and the third site of twelve-fold disordered solvent (crystallographically independent are one thf position and two pentane positions). The main molecule also shows disorder, namely one of the two independent adamantly ligands as well as both CHCMe₂Ph ligands were modeled as disordered over two positions. The eight-fold disordered pentane (described above as the second site) interferes with one orientation of each of the disordered CHCMe₂Ph ligands and the occupancies of the disorder components were linked accordingly. In addition to the similarity restraints mentioned above, all disordered solvent atoms were restrained to behave approximately isotropically within 0.1 Å (0.2 Å for terminal atoms). Even though all attempts to resolve more disorders failed, larger than average thermal parameter for all atoms suggest high molecular motion and probably more disorders. It also seems likely that the solvent disorder model, although fairly complex, is still not reflecting all aspects of the actual solvent situation and a bulk solvent correction based on Babinet's principle was applied (P. C. Moews, R. H. Kretsinger, J. Mol. Biol. 1975, 91, 201-228). The circumstance that the structure contains only fractions of solvent molecules per octamer leads to non-integer values in the empirical formula for the elements C, H and O.

Compound 2a:

Compound 2a crystallizes in the triclinic space group P-1 with one molecule of 2a and half a molecule of pentane in the asymmetric unit. The pentane molecule is located close to a crystallographic inversion center and disordered over four positions, two of which are crystallographically independent. In addition to the similarity restraints mentioned above, the disordered solvent atoms were restrained to behave approximately isotropically within 0.1 Å (0.2 Å for terminal atoms). Coordinates for the hydrogen atom on Cl, the carbon binding directly to Mo, were taken from the difference Fourier synthesis. This hydrogen atom was subsequently refined semi-freely with the help of a distance restraint while constraining its Uᵢₛₒ to 1.2 times the U_{eq} value of C1. The circumstance that the structure contains only half a pentane per molecule of 2a leads to a non-integer value in the empirical formula for the element C.

Compound 2b:

Compound 2b crystallizes in the monoclinic space group P2₁ with one molecule of 2b in the asymmetric unit. Coordinates for the hydrogen atom on Cl, the carbon binding directly to Mo, were taken from the difference Fourier synthesis. This hydrogen atom was subsequently refined semi-freely with the help of a distance restraint while constraining its Uᵢₛₒ to 1.2 times the U_{eq} value of Cl. The Flack x parameter refined to −0.006(3) (Flack H. D., Acta Cryst. 1983 A39, 876-881).

Compound 2c:

Compound 2c crystallizes in the monoclinic space group C2/c with one molecule of 2c and half a molecule of tetramethylsilane (tms) in the asymmetric unit. The tms molecule is located close to but not on a crystallographic two-fold axis and disordered accordingly over two positions. The alkoxide ligand was modeled as a two part disorder. The ratio was refined freely and converged at 0.710(3). In addition to the similarity restraints mentioned above some almost overlapping atoms were pair-wise constrained to have identical anisotropic displacement parameters (O1/O1A, C41/C41A, C42/C42A, and C46/C46A). Coordinates for the hydrogen atom on Cl, the carbon binding directly to Mo, were taken from the difference Fourier synthesis. This hydrogen atom was subsequently refined semi-freely with the help of a distance restraint while constraining its Uᵢₛₒ to 1.2 times the U_{eq} value of C1. The circumstance that the structure contains only half a tms per molecule of 2c leads to a non-integer value in the empirical formula for the element Si.

Compound 3(PMe₃):

Compound 3(PMe₃) crystallizes in the monoclinic space group P2₁/n with one molecule of 3(PMe₃) in the asymmetric unit. Coordinates for the hydrogen atom on Cl, the carbon binding directly to Mo, were taken from the difference Fourier synthesis. This hydrogen atom was subsequently refined semi-freely with the help of a distance restraint while constraining its Uᵢₛₒ to 1.2 times the U_{eq} value of Cl. One of the ⁱPr groups was treated as disordered over two positions.

Compound 4:

Compound 4 crystallizes in the monoclinic space group P2₁/c with one molecule of 4 in the asymmetric unit. Coordinates for the hydrogen atom on Cl, the carbon binding directly to Mo, were taken from the difference Fourier synthesis. This hydrogen atom was subsequently refined semi-freely with the help of a distance restraint while constraining its Uᵢₛₒ to 1.2 times the U_{eq} value of Cl. Two of the ⁱPr groups were treated as disordered over two positions. The highest residual electron density maximum in the difference Fourier map was significantly higher than all other maxima (7.0 electrons per Å³, compared to 1.1 electrons for second highest peak). This maximum was located 0.82 Å away from the Mo position and the deepest electron density hole (−2.7 electrons per Å³) was located 0.58 Å from Mo1, suggesting a second position for the metal atom. Upon careful examination of the difference Fourier synthesis, alternative positions for the ligand atoms could also be distinguished, however a refinement of the whole-molecule disorder was not stable. Therefore the final model contains only the second Mo position; the ratio between first and second component of this incomplete whole-molecule disorder was refined freely and converged at 0.849 (6). Introduction of the second Mo site improved the model significantly: the R1 dropped by over three points and the highest residual electron density maximum is down to 0.75 electrons.

Compound 5:

Compound 5 crystallizes in the triclinic space group P-1 with two molecules of 5 in the asymmetric unit. One of these two molecules shows extensive disorder, which was treated as described above. The other molecule shows no disorder and the discussion of the structure of 5 in the main text is limited to the well-behaved molecule. Coordinates for the hydrogen atoms on Cl and C101, the carbon atoms binding directly to the two Mo centers, were taken from the difference Fourier synthesis. These hydrogen atoms were subsequently refined semi-freely with the help of a distance restraints while constraining their Uᵢₛₒ to 1.2 times the U_{eq} value of Cl or C101, respectively. The crystal was non-merohedrally twinned. Two independent orientation matrices for the unit cell were found using the program CELL_NOW (Sheldrick, G. M (2008). CELL_NOW, University of Gottingen, Germany), and data reduction taking into account the twinning was performed with SAINT (Bruker (2010). SAINT, Bruker-AXS Inc., Madison, Wis., USA). The program TWINABS (Sheldrick, G. M (2008). TWINABS, University of Gottingen, Germany) was used to perform absorption correction and to set up the HKLF5 format file for structure refinement. The twin ratio was refined freely and converged at a value of 0.4642(6).

Compound 6d:

Compound 6d crystallizes in the triclinic space group P-1 with one molecule of 6d in the asymmetric unit. Coordinates for the hydrogen atom on Cl, the carbon binding directly to Mo, were taken from the difference Fourier synthesis. This hydrogen atom was subsequently refined semi-freely with the help of a distance restraint while constraining its Uᵢₛₒ to 1.2 times the U_{eq} value of Cl. The crystal was non-merohedrally twinned which was addressed as described for the structure of compound 5. The twin ratio was refined freely and converged at a value of 0.0916(4).

Compound 7b:

Compound 7b crystallizes in the monoclinic space group P2₁/c with one molecule of 7b in the asymmetric unit. Coordinates for the hydrogen atoms on Cl, the carbon atom binding directly to Mo, and N2 were taken from the difference Fourier synthesis. These hydrogen atoms were subsequently refined semi-freely with the help of a distance restraints while constraining their Uᵢₛₒ to 1.2 times the U_{eq} value of Cl or N2, respectively.

Compound 8:

Compound 8 crystallizes in the triclinic space group P-1 with two molecules of 8 in the asymmetric unit. In both independent molecules, there is a three part disorder for the alkoxide ligand, approximately corresponding to a rotation of the C(CF₃)₂(CH₃) group about the Mo—O bond. The anisotropic displacement parameters of the F and C atoms in the CF₃ groups were pairwise constrained to be identical. The crystal was non-merohedrally twinned which was addressed as described for the structure of compound 5. The twin ratio was refined freely and converged at a value of 0.3032(17).

TABLE 3
Crystal data and structure refinement for 1b.

| Identification code | d10066 | |
|---|---|---|
| Empirical formula | C253.45 H301.80 Mo8 N40 O1.55 | |
| Formula weight | 4700.87 | |
| Temperature | 100(2) K | |
| Wavelength | 1.54178 Å | |
| Crystal system | Tetragonal | |
| Space group | P4/ncc | |
| Unit cell dimensions | a = 28.5841(4) Å | α = 90° |
| | b = 28.5841(4) Å | β = 90° |
| | c = 31.1532(7) Å | γ = 90° |

| | |
|---|---|
| Volume | 25453.7(8) Å³ |
| Z | 4 |
| Density (calculated) | 1.227 Mg/m³ |
| Absorption coefficient | 3.570 mm⁻¹ |
| F(000) | 9804 |
| Crystal size | 0.40 × 0.15 × 0.10 mm³ |
| Theta range for data collection | 2.19 to 61.16° |
| Index ranges | −32 <= h <= 31, −32 <= |
| | k <= 32, −35 <= l <= 35 |
| Reflections collected | 385460 |
| Independent reflections | 9789 [R(int) = 0.1364] |
| Completeness to theta = 61.16° | 100.0% |
| Absorption correction | Semi-empirical from equivalents |
| Max. and min. transmission | 0.7166 and 0.3293 |
| Refinement method | Full-matrix least-squares on F² |
| Data/restraints/parameters | 9789/2725/1149 |
| Goodness-of-fit on F² | 1.070 |
| Final R indices [I > 2sigma(I)] | R1 = 0.0724, wR2 = 0.1999 |
| R indices (all data) | R1 = 0.1151, wR2 = 0.2503 |
| Largest diff. peak and hole | 0.740 and −0.499 e.Å⁻³ |

TABLE 4
Crystal data and structure refinement for 2c.

| Identification code | X8_10082 | |
|---|---|---|
| Empirical formula | C71 H96 Mo N2 O Si0.50 | |
| Formula weight | 1103.48 | |
| Temperature | 100(2) K | |
| Wavelength | 0.71073 Å | |
| Crystal system | Monoclinic | |
| Space group | C2/c | |
| Unit cell dimensions | a = 41.846(2) Å | α = 90° |
| | b = 14.9408(7) Å | β = 96.4610(10)° |
| | c = 20.0700(10) Å | γ = 90° |

| | |
|---|---|
| Volume | 12468.2(11) Å³ |
| Z | 8 |
| Density (calculated) | 1.176 Mg/m³ |
| Absorption coefficient | 0.263 mm⁻¹ |
| F(000) | 4744 |
| Crystal size | 0.20 × 0.20 × 0.10 mm³ |
| Theta range for data collection | 1.45 to 30.31° |
| Index ranges | −59 <= h <= 59, −21 <= |
| | k <= 21, −28 <= l <= 28 |
| Reflections collected | 145253 |
| Independent reflections | 18713 [R(int) = 0.0718] |
| Completeness to theta = 30.31° | 100.0% |
| Absorption correction | Semi-empirical from equivalents |
| Max. and min. transmission | 0.9742 and 0.9492 |
| Refinement method | Full-matrix least-squares on F² |
| Data/restraints/parameters | 18713/1384/1020 |
| Goodness-of-fit on F² | 1.017 |
| Final R indices [I > 2sigma(I)] | R1 = 0.0367, wR2 = 0.0800 |
| R indices (all data) | R1 = 0.0582, wR2 = 0.0903 |
| Largest diff. peak and hole | 0.439 and −0.498 e.Å⁻³ |

TABLE 5
Crystal data and structure refinement for 3(PMe₃).

| Identification code | 11015 | |
|---|---|---|
| Empirical formula | C60H85F3MoNO4PS | |
| Formula weight | 1100.26 | |
| Temperature | 100(2) K | |
| Wavelength | 0.71073 Å | |
| Crystal system | Monoclinic | |
| Space group | P2₁/n | |
| Unit cell dimensions | a = 11.9252(15) Å | α = 90° |
| | b = 23.200(3) Å | β = 99.781(2)° |
| | c = 20.709(3) Å | γ = 90° |

| | |
|---|---|
| Volume | 5646.0(13) Å³ |
| Z | 4 |
| Density (calculated) | 1.294 Mg/m³ |
| Absorption coefficient | 0.353 mm⁻¹ |
| F(000) | 2336 |
| Crystal size | 0.30 × 0.20 × 0.15 mm³ |
| Theta range for data collection | 1.33 to 30.32° |
| Index ranges | −16 <= h <= 16, −32 <= k <= 32, |
| | −29 <= l <= 29 |
| Reflections collected | 156288 |
| Independent reflections | 16915 [R(int) = 0.0730] |
| Completeness to theta = 30.32° | 99.9% |
| Absorption correction | Semi-empirical from equivalents |
| Max. and min. transmission | 0.9489 and 0.9014 |
| Refinement method | Full-matrix least-squares on F² |
| Data/restraints/parameters | 16915/56/677 |
| Goodness-of-fit on F² | 1.041 |
| Final R indices [I > 2sigma(I)] | R1 = 0.0345, wR2 = 0.0790 |
| R indices (all data) | R1 = 0.0473, wR2 = 0.0864 |
| Largest diff. peak and hole | 0.577 and −0.750 e · Å⁻³ |

TABLE 6
Crystal data and structure refinement for 4.

| Identification code | X8_11002 | |
|---|---|---|
| Empirical formula | C61H79MoN3O | |
| Formula weight | 966.21 | |
| Temperature | 100(2) K | |
| Wavelength | 0.71073 Å | |
| Crystal system | Monoclinic | |
| Space group | P2₁/c | |
| Unit cell dimensions | a = 22.5368(13) Å | α = 90° |
| | b = 12.3855(7) Å | β = 106.3860(10)° |
| | c = 19.7104(12) Å | γ = 90° |

| | |
|---|---|
| Volume | 5278.3(5) Å³ |
| Z | 4 |
| Density (calculated) | 1.216 Mg/m³ |
| Absorption coefficient | 0.291 mm⁻¹ |
| F(000) | 2064 |
| Crystal size | 0.10 × 0.10 × 0.05 mm³ |
| Theta range for data collection | 1.88 to 29.13° |
| Index ranges | −30 <= h <= 30, −16 <= k <= 16, |
| | −26 <= l <= 26 |
| Reflections collected | 118187 |
| Independent reflections | 14184 [R(int) = 0.0849] |
| Completeness to | 99.9% |
| theta = 29.13° | |
| Absorption correction | Semi-empirical from equivalents |
| Max. and min. transmission | 0.9856 and 0.9715 |
| Refinement method | Full-matrix least-squares on F² |
| Data/restraints/parameters | 14184/191/646 |
| Goodness-of-fit on F² | 1.127 |
| Final R indices | R1 = 0.0622, wR2 = 0.1539 |
| [I > 2sigma(I)] | |
| R indices (all data) | R1 = 0.0817, wR2 = 0.1634 |
| Largest diff. peak and hole | 0.750 and −0.726 e · Å⁻³ |

TABLE 7
Crystal data and structure refinement for 5.

| Identification code | X8_10084_t5 | |
|---|---|---|
| Empirical formula | C₆₀H₈₅MoNO₂ | |
| Formula weight | 948.23 | |
| Temperature | 100(2) K | |
| Wavelength | 0.71073 Å | |
| Crystal system | Triclinic | |
| Space group | P1 | |
| Unit cell dimensions | a = 12.0586(14) Å | α = 89.536(3)° |
| | b = 13.0094(14) Å | β = 88.685(3)° |
| | c = 37.249(4) Å | γ = 64.701(3)° |

| | |
|---|---|
| Volume | 5281.5(10) Å³ |
| Z | 4 |
| Density (calculated) | 1.193 Mg/m³ |
| Absorption coefficient | 0.290 mm⁻¹ |
| F(000) | 2040 |
| Crystal size | 0.15 × 0.10 × 0.10 mm³ |
| Theta range for data collection | 1.64 to 30.60° |
| Index ranges | −17 <= h <= 17, −18 <= k <= 18, |
| | 0 <= l <= 53 |
| Reflections collected | 33165 |
| Independent reflections | 33171 [R(int) = 0.0799] |
| Completeness to theta = 30.60° | 97.6% |
| Absorption correction | Semi-empirical from equivalents |
| Max. and min. transmission | 0.9716 and 0.9578 |
| Refinement method | Full-matrix least-squares on F² |
| Data/restraints/parameters | 33171/896/1306 |
| Goodness-of-fit on F² | 1.088 |
| Final R indices [I > 2sigma(I)] | R1 = 0.0586, wR2 = 0.1311 |
| R indices (all data) | R1 = 0.0712, wR2 = 0.1366 |
| Largest diff. peak and hole | 1.338 and −1.105 e · Å⁻³ |

TABLE 8
Crystal data and structure refinement for 6d.

| Identification code | X8_12044 | |
|---|---|---|
| Empirical formula | C48H55F6MoNO2 | |
| Formula weight | 887.87 | |
| Temperature | 100(2) K | |
| Wavelength | 0.71073 Å | |
| Crystal system | Triclinic | |
| Space group | P1 | |
| Unit cell dimensions | a = 11.3519(5) Å | α = 102.7680(10)° |
| | b = 11.6306(6) Å | β = 95.3350(10)° |
| | c = 18.6057(9) Å | γ = 112.9690(10)° |

| | |
|---|---|
| Volume | 2161.43(18) Å³ |
| Z | 2 |
| Density (calculated) | 1.364 Mg/m³ |
| Absorption coefficient | 0.368 mm⁻¹ |
| F(000) | 924 |
| Crystal size | 0.23 × 0.22 × 0.10 mm³ |
| Theta range for data collection | 1.98 to 31.15° |
| Index ranges | −16 <= h <= 16, −16 <= k <= 16, |
| | 0 <= l <= 27 |
| Reflections collected | 13841 |
| Independent reflections | 13841 [R(int) = 0.0495] |
| Completeness to theta = 31.15° | 99.2% |
| Absorption correction | Semi-empirical from equivalents |
| Max. and min. transmission | 0.9641 and 0.9201 |
| Refinement method | Full-matrix least-squares on F² |
| Data/restraints/parameters | 13841/49/536 |
| Goodness-of-fit on F² | 1.043 |
| Final R indices [I > 2sigma(I)] | R1 = 0.0354, wR2 = 0.0943 |
| R indices (all data) | R1 = 0.0374, wR2 = 0.0958 |
| Largest diff. peak and hole | 0.894 and −1.196 e · Å⁻³ |

TABLE 9
Crystal data and structure refinement for 7b.

| Identification code | X8_12055 | |
|---|---|---|
| Empirical formula | C47H52F6MoN2O | |
| Formula weight | 870.85 | |
| Temperature | 100(2) K | |
| Wavelength | 0.71073 Å | |
| Crystal system | Monoclinic | |
| Space group | P2(1)/c | |
| Unit cell dimensions | a = 20.7835(19) Å | α = 90° |
| | b = 10.8355(10) Å | β = 104.390(2)° |
| | c = 19.7836(18) Å | γ = 90° |

| | |
|---|---|
| Volume | 4315.5(7) Å³ |
| Z | 4 |
| Density (calculated) | 1.340 Mg/m³ |
| Absorption coefficient | 0.366 mm⁻¹ |
| F(000) | 1808 |
| Crystal size | 0.06 × 0.05 × 0.02 mm³ |
| Theta range for data collection | 2.02 to 31.00° |
| Index ranges | −30 <= h <= 30, −15 <= k <= 15, |
| | −28 <= l <= 28 |
| Reflections collected | 205294 |
| Independent reflections | 13705 [R(int) = 0.0537] |
| Completeness to theta = 31.00° | 99.6% |
| Absorption correction | Semi-empirical from equivalents |
| Max. and min. transmission | 0.9927 and 0.9783 |
| Refinement method | Full-matrix least-squares on F² |
| Data/restraints/parameters | 13705/2/531 |
| Goodness-of-fit on F² | 1.061 |
| Final R indices [I > 2sigma(I)] | R1 = 0.0327, wR2 = 0.0786 |
| R indices (all data) | R1 = 0.0432, wR2 = 0.0836 |
| Largest diff. peak and hole | 1.422 and −0.642 e · Å⁻³ |

TABLE 10
Crystal data and structure refinement for 8.

| Identification code | X8_11109_t5 | |
|---|---|---|
| Empirical formula | C48H54F6MoNO | |
| Formula weight | 870.86 | |
| Temperature | 100(2) K | |
| Wavelength | 0.71073 Å | |
| Crystal system | Triclinic | |
| Space group | P1 | |
| Unit cell dimensions | a = 10.6178(9) Å | α = 109.268(2)° |
| | b = 18.8500(16) Å | β = 96.928(2)° |
| | c = 22.2527(19) Å | γ = 90.162(2)° |

| | |
|---|---|
| Volume | 4169.3(6) Å³ |
| Z | 4 |
| Density (calculated) | 1.387 Mg/m³ |
| Absorption coefficient | 0.379 mm⁻¹ |
| F(000) | 1812 |
| Crystal size | 0.20 × 0.15 × 0.05 mm³ |
| Theta range for data collection | 1.15 to 30.62° |
| Index ranges | −15 <= h <= 15, −26 <= k <= 25, |
| | 0 <= l <= 31 |
| Reflections collected | 23776 |
| Independent reflections | 23776 [R(int) = 0.0958] |
| Completeness to theta = 25.00° | 98.0% |
| Absorption correction | Semi-empirical from equivalents |
| Max. and min. transmission | 0.9813 and 0.9281 |
| Refinement method | Full-matrix least-squares on F² |
| Data/restraints/parameters | 23538/3839/1265 |
| Goodness-of-fit on F² | 1.051 |
| Final R indices [I > 2sigma(I)] | R1 = 0.0764, wR2 = 0.1836 |
| R indices (all data) | R1 = 0.1030, wR2 = 0.2015 |
| Largest diff. peak and hole | 3.265 and −2.969 e · Å⁻³ |

### Synthesis of Exemplary Compounds and their Uses Thereof

### Synthesis of Catalyst A1

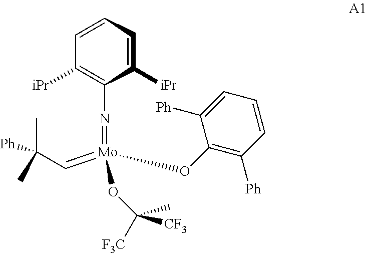

Experimental:

All manipulations were performed under the inert atmosphere of glovebox. To the corresponding bispyrrolide precursor (N-[bis(2,5-dimethyl-1H-pyrrol-1-yl)(2-methyl-2-phenylpropylidene)molybdenumylidene]-2,6-bis(propan-2-yl)aniline) (0.035 mmol, 20.7 mg) dissolved in 350 μmoL C₆D₆ 1 eq. (0.035 mmol, 6.37 mg, 4.29 μL) hexafluoro-tert-butanol in 350 μL C₆D₆ was added dropwise at ambient temperature and stirred at the same temperature. After 2 hours 1 eq. 2,6-diphenyl-phenol (0.035 mmol, 8.62 mg) was added in one portion and stirred for 2 h at 80° C.

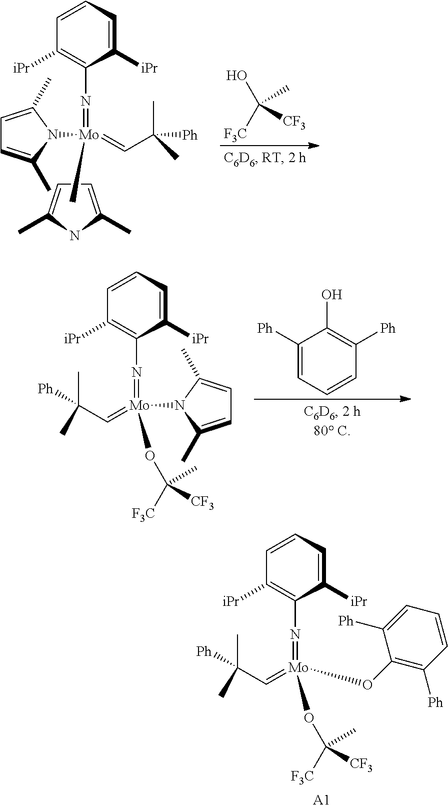

Alkylidene ¹H Signal (C₆D₆):

Product: 11.58 ppm (s); possible impurity: bis(hexafluoro-tert-butoxide) complex: 12.09 ppm (s, absent); impurity: bis(diphenyl-phenoxy) complex: 11.44 ppm (s, less than 1%).

### Synthesis of Catalyst A2

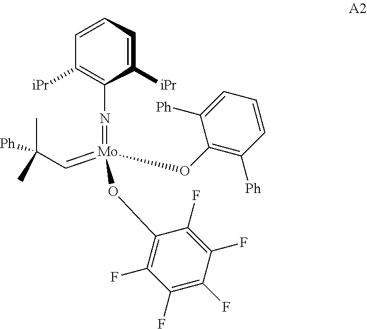

Experimental:

All manipulations were performed under the inert atmosphere of glovebox. To the corresponding bispyrrolide precursor (N-[bis(2,5-dimethyl-1H-pyrrol-1-yl)(2-methyl-2-phenylpropylidene)molybdenumylidene]-2,6-bis(propan-2-yl)aniline) (0.075 mmol, 44.4 mg) dissolved in 300 μL C₆D₆ 1 eq. (0.075 mmol, 18.5 mg) 2,6-diphenyl-phenol in 300 μL C₆D₆ was added dropwise at ambient temperature and stirred at the same temperature. After 2 h stirring 1 eq. pentafluoro-phenol (0.075 mmol, 13.8 mg) in 200 μL C₆D₆ was added in one portion and stirred overnight at ambient temperature. Finally, the heterogenous mixture was diluted to 3 mL using benzene.

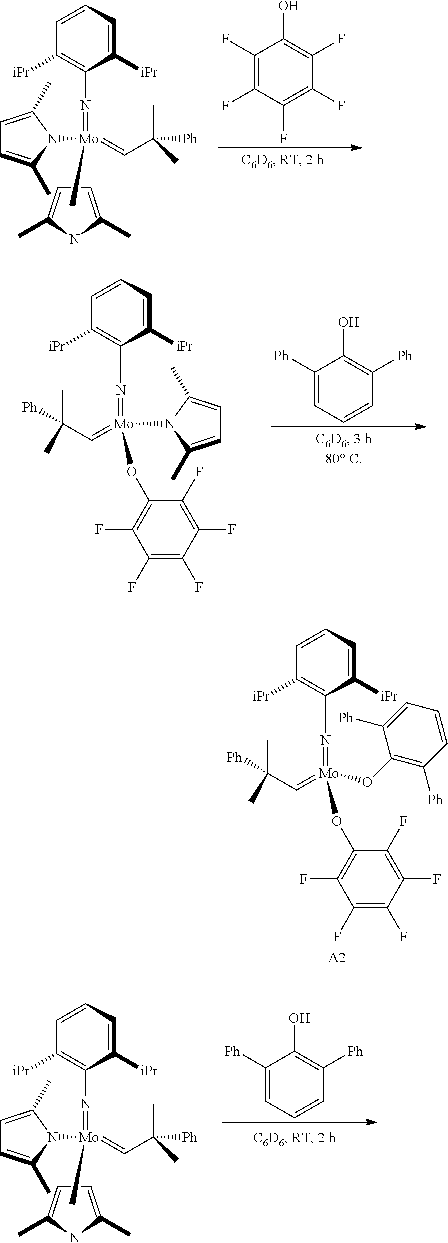

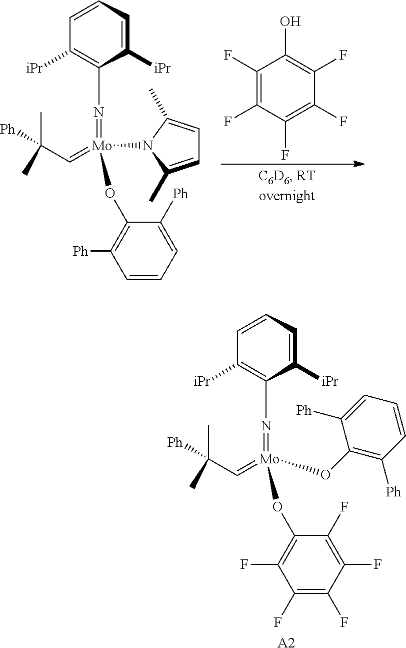

Alkylidene ¹H Signal (C₆D₆):

Product: 11.72 ppm (s); Possible impurity: bis(pentafluoro-phenoxy) complex: unknown (not present, insoluble in benzene or in benzene-DCM mixture); Impurity: bis(diphenyl-phenoxy) complex: 11.44 ppm (s, less than 1%)

### Synthesis of Catalyst A3

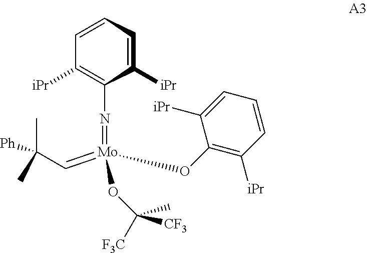

IUPAC Name:

N-[2,6-bis(propan-2-yl)phenoxy[(1,1,1,3,3,3-hexafluoro-2-methylpropan-2-yl)oxy](2-ethyl-2-phenylpropylidene)molybdenumylidene]-2,6-bis(propan-2-yl)aniline.

Chemical Formula:

C₃₈H₄₉F₆MoNO₂

Molecular Weight:

761.75

Experimental:

All manipulations were performed under the inert atmosphere of glovebox. To the corresponding bispyrrolide precursor (N-[bis(2,5-dimethyl-1H-pyrrol-1-yl)(2-methyl-2-phenylpropylidene)molybdenumylidene]-2,6-bis(propan-2-yl)aniline) (0.1 mmol, 59.2 mg) dissolved in 500 μL, C₆D₆ 1 eq. (0.1 mmol, 18.2 mg, 12.3 μL) hexafluoro-tert-butanol in 500 μL C₆D₆ was added dropwise at ambient temperature and stirred at the same temperature. After 2 hours 1 eq. 2,6-diisopropyl-phenol (0.1 mmol, 17.8 mg, 18.5 μL) was added in one portion and stirred overnight.

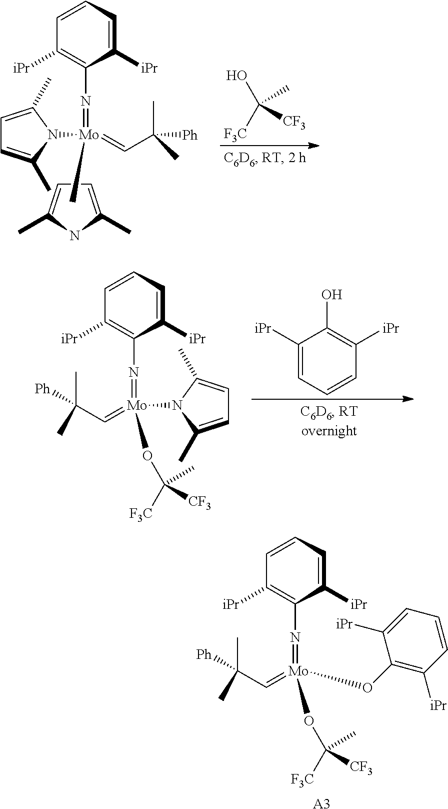

Alkylidene ¹H Signal (C₆D₆):

Product (3): 11.91 ppm (s, 68%); Impurity (A): bis(diisopropyl-phenoxy) complex: 11.72 ppm (s, 19%); Impurity (B): bis(hexafluoro-tert-butoxide) complex: 12.09 ppm (s, 13%); Remark: If the order of substituent introduction into the complex were reverted, and second step were performed for 2 h, the product composition was of 90% 3, 6% A, 3% B.

### Synthesis of Catalyst A4

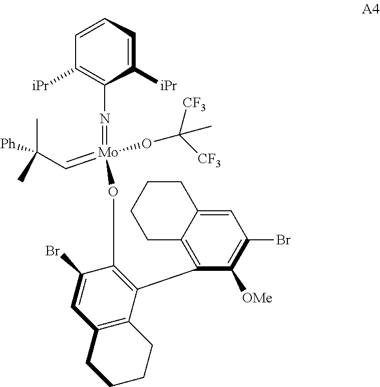

IUPAC Name:

N-({[3-bromo-1-(3-bromo-2-methoxy-5,6,7,8-tetrahydronaphthalen-1-yl)-5,6,7,8-tetrahydronaphthalen-2-yl]oxy} [(1,1,1,3,3,3-hexafluoro-2-methylpropan-2-yl)oxy](2-methyl-2-phenylpropylidene)molybdenumylidene)-2,6-bis(propan-2-yl)aniline

Chemical Formula:

C₄₇H₅₃Br₂F₆MoNO₃

Molecular Weight:

1049.69

Experimental:

All manipulations were performed under the inert atmosphere of glovebox. To the corresponding bispyrrolide precursor (N-[bis(2,5-dimethyl-1H-pyrrol-1-yl)(2-methyl-2-phenylpropylidene)molybdenumylidene]-2,6-bis(propan-2-yl)aniline) (0.1 mmol, 59.2 mg) dissolved in 500 μL, C₆D₆ 1 eq. (0.1 mmol, 18.2 mg, 12.3 μL) hexafluoro-tert-butanol in 500 μL C₆D₆ was added dropwise at ambient temperature and stirred at the same temperature. After hours 1 eq. (0.1 mmol, 45.8 mg) 3-bromo-1-(3-bromo-2-methoxy-5,6,7,8-tetrahydronaphthalen-1-yl)-5,6,7,8-tetrahydronaphthalen-2-ol in 500 μL C₆D₆ was added dropwise at ambient temperature and stirred overnight.

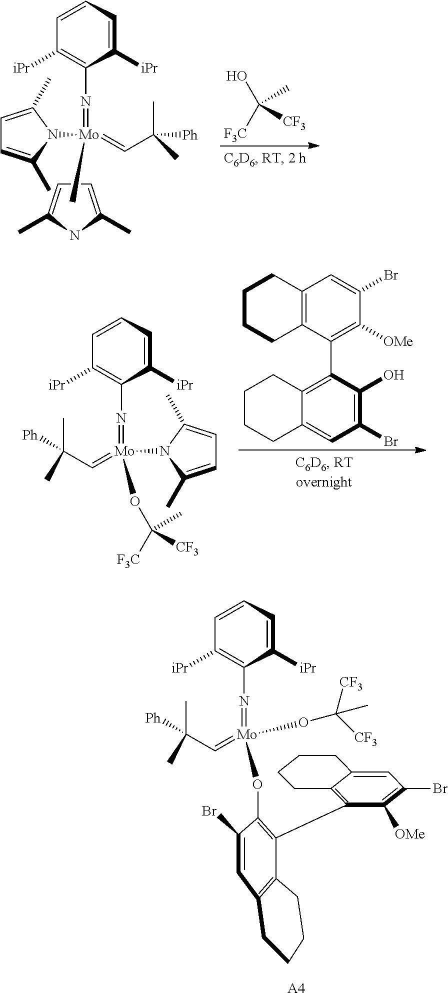

Alkylidene ¹H Signal (C₆D₆):

Product: 12.20 and 12.27 ppm (diastereomers, 48% and 39%, respectively, total: 87%); Impurity: 12.74 ppm (3%); Impurity: bis(hexafluoro-tert-butoxide) complex: 12.09 ppm (s, 9%). Remark: If the order of substituent introduction into the complex were reverted, and second step were performed for 2 h, the product composition was similar to the above described.

### Synthesis of Catalyst A5

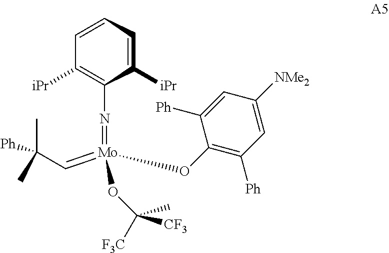

Experimental:

All manipulations were performed under the inert atmosphere of glovebox. To the corresponding bispyrrolide precursor (N-[bis(2,5-dimethyl-1H-pyrrol-1-yl)(2-methyl-2-phenylpropylidene)molybdenumylidene]-2,6-bis(propan-2-yl)aniline) (0.035 mmol, 20.7 mg) dissolved in 350 μL C₆D₆ 1 eq. (0.035 mmol, 6.37 mg, 4.29 μL) hexafluoro-tert-butanol in 350 μL C₆D₆ was added dropwise at ambient temperature and stirred at the same temperature. After 2 hours 1 eq. 2,6-diphenyl-4-dimethylaminophenol (0.035 mmol) was added in one portion and stirred for overnight at room temperature.

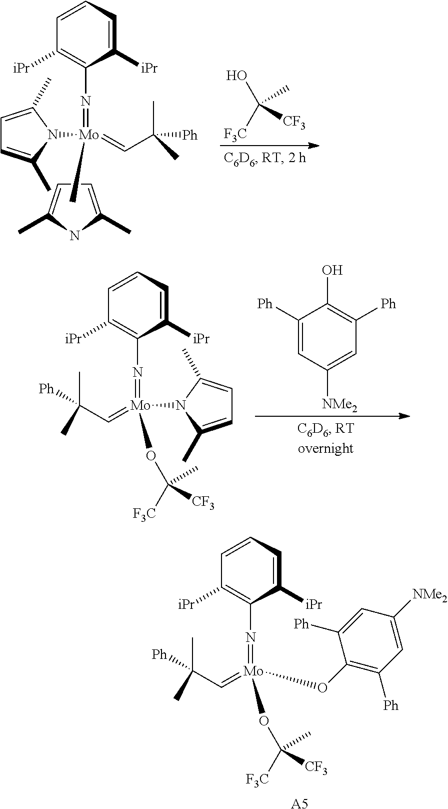

Alkylidene ¹H Signal (C₆D₆):

Product: 11.58 ppm (s, more than 99%); Impurity: bis(hexafluoro-tert-butoxide) complex: 12.09 ppm (s, less than 1%); Impurity: bis(dimethylamino-diphenyl-phenoxy) complex: 11.27 ppm (s, less than 1%).

### Synthesis of Catalyst A6

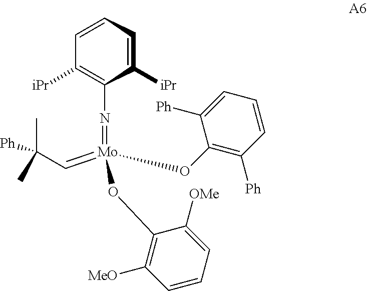

IUPAC Name:

N-[2,6-dimethoxyphenoxy(2,6-diphenylphenoxy)(2-methyl-2-phenylpropylidene)molybdenumylidene]-2,6-bis(propan-2-yl)aniline

Chemical Formula:

C₄₈H₅₁MoNO₄

Molecular Weight:

801.88

Experimental:

All manipulations were performed under the inert atmosphere of glovebox. To the corresponding bispyrrolide precursor (N-[bis(2,5-dimethyl-1H-pyrrol-1-yl)(2-methyl-2-phenylpropylidene)molybdenumylidene]-2,6-bis(propan-2-yl)aniline) (0.056 mmol, 33.0 mg) dissolved in 500 μL C₆D₆ 1 eq. (0.056 mmol, 8.63 mg) 2,6-dimethoxyphenol in 60 μL C₆D₆ was added dropwise at ambient temperature and stirred at the same temperature. After 2 hours 1 eq. 2,6-diphenyl-phenol (0.056 mmol, 13.8 mg) was added in one portion and stirred for 20 h at room temperature.

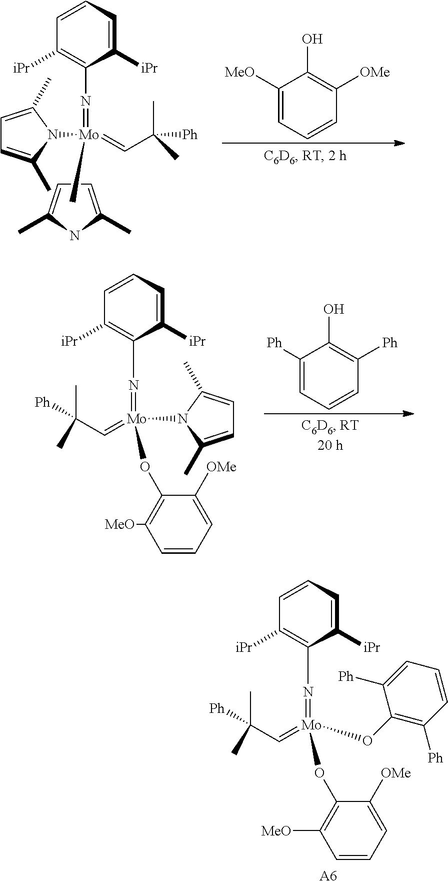

Alkylidene ¹H Signal (C₆D₆):

Product: 11.20 ppm (s, 70%); Impurity: bis(dimethoxyphenoxy) complex: 11.85 ppm (s, 27%); Impurity: bis(diphenylphenoxy) complex: 11.48 ppm (s, 3%).

### Synthesis of Catalyst A7

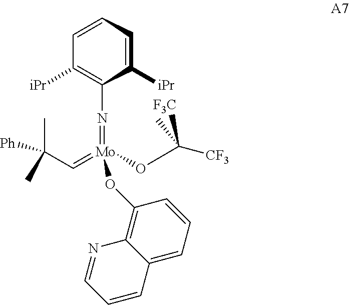

IUPAC Name:

N-{[(1,1,1,3,3,3-hexafluoro-2-methylpropan-2-yl)oxy](2-methyl-2-phenylpropylidene)(quinolin-8-yloxy)molybdenumylidene}-2,6-bis(propan-2-yl)aniline

Chemical Formula:

C₃₅H₃₈F₆MoN₂O₂

Molecular Weight:

728.64

Experimental:

All manipulations were performed under the inert atmosphere of glovebox. To the corresponding bispyrrolide precursor (N-[bis(2,5-dimethyl-1H-pyrrol-1-yl)(2-methyl-2-phenylpropylidene)molybdenumylidene]-2,6-bis(propan-2-yl)aniline) (0.056 mmol, 33.0 mg) dissolved in 500 μL C₆D₆ 1 eq. (0.056 mmol, 8.1 mg) 8-hydroxyquinoline in 60 μL C₆D₆ was added dropwise at ambient temperature and stirred at the same temperature. After 2 hours 1 eq. hexafluoro-tert-butanol (0.056 mmol, 10.2 mg, 6.85 μL) was added in one portion and stirred for 2 h at room temperature.

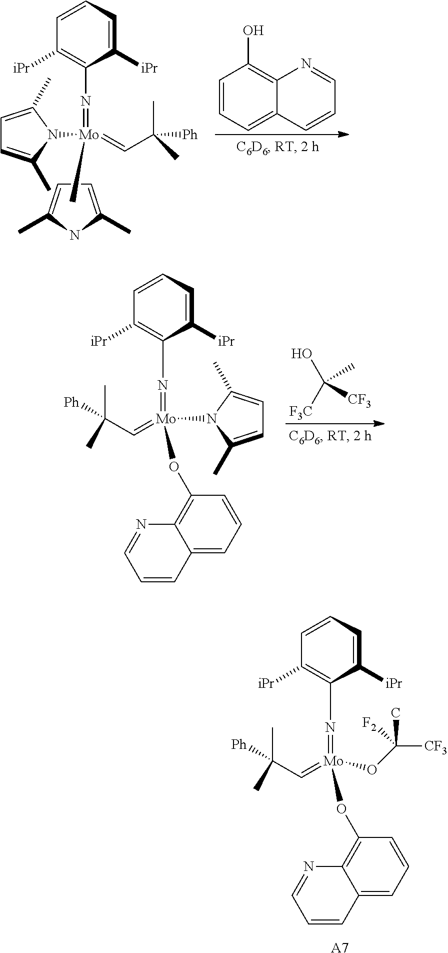

Alkylidene ¹H Signal (C₆D₆):

Product: 12.63 and 13.66 ppm (together 75%); Impurity: 12.06 ppm quinolin-8-yloxy-dimethylpyrrolide complex (12.06 ppm, 25%).

Use of Catalyst A1 in Self-Metathesis of Methyl-Dec-9-Enoate

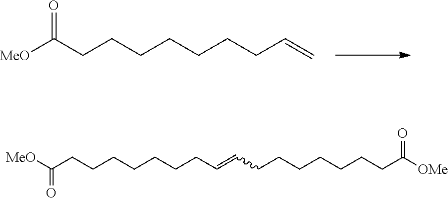 [SMILES: C=CCCCCCCCC(=O)OC ; COC(=O)CCCCCCCC=CCCCCCCCC(=O)OC]

Reaction Type:

Methyl dec-9-enoate self-metathesis

Catalyst: A1

Experimental:

Pretreatment: Methyl-dec-9-enoate was taken into glovebox, percolated on an alumina pad of 20% alumina and stored over 3 Å molecular sieve.

In a 4 mL glass vial equipped with a perforated cap and a magnetic stirbar 330 μL (1.58 mmol) pretreated methyl-9-decenote was filled together with 1 μmol catalyst A1 (as 0.05 M stock solution in benzene-d6, 20 μL). After 4 h stirring at room temperature ca. 100 μL sample was taken out of the glovebox, measured by weight, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion^{b} | Yield^{c} | | |
|---|---|---|---|---|
| mol % | % | % | TON^{d} | Z/E^{e} |
| 0.06 | 89 | 89 | 446 | 21/79 |

^{a}moles of catalyst A1/moles of methyl-dec-9-enoate × 100
^{b}100 × (1 − moles of methyl-dec-9-enoate in product/initial moles of methyl-dec-9-enoate)
^{c}moles of dimethyl-octadec-9-enedioate/initial moles of methyl-dec-9-enoate × 50
^{d}moles of dimethyl-octadec-9-enedioate/moles of catalyst A1 × 2
^{e}ratio of (Z)- and (E)-dimethyl-octadec-9-enedioate

Use of Catalyst A1 in Self-Metathesis of Allylbenzene

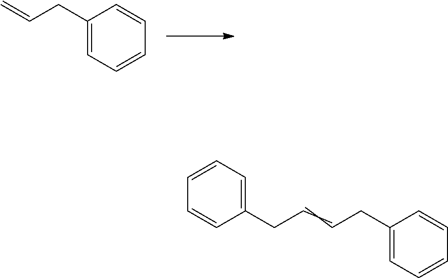

Reaction Type:

Allylbenzene self-metathesis

Catalyst: A1

Experimental:

Pretreatment: Allylbenzene was taken into glovebox, percolated on an alumina pad of 20% alumina and stored over 3 Å molecular sieve prior use.

In a 4 mL glass vial equipped with a perforated cap and a magnetic stirbar 265 μL (2 mmol) pretreated allylbenzene was filled together with 2 μmol A1 catalyst in (as 0.05 M stock solution in benzene-d6, 40 μL). After 4 h stirring at room temperature ca. 100 μL sample was taken out of the glovebox, measured by weight, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion^{b} | Yield^{c} | | |
|---|---|---|---|---|
| mol % | % | % | TON^{d} | Z/E^{e} |
| 0.1 | 47 | 47 | 233 | 47/53 |

^{a}moles of catalyst A1/initial moles of allylbenzene × 100
^{b}100 × (1 − moles of allylbenzene in product/initial moles of allylbenzene)
^{c}moles of 1,4-diphenyl-but-2-ene/initial moles of allylbenzene × 50
^{d}moles of 1,4-diphenyl-but-2-ene/moles of catalyst A1 × 2
^{e}ratio of (Z)- and (E)-1,4-diphenyl-but-2-ene

Use of Catalyst A1 in RCM of Diethyl-Diallyl-Malonate

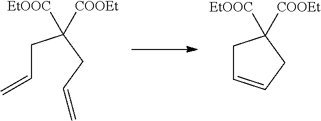 [SMILES: C=CCC(CC=C)(C(=O)OCC)C(=O)OCC ; CCOC(=O)C1(C(=O)OCC)CC=CC1]

Reaction Type:

Diethyl-diallyl-malonate ring closing metathesis

Catalyst: A1

Experimental:

Pretreatment: Diethyl-diallyl-malonate was taken into glovebox, percolated on an alumina pad of 20% alumina and stored over 3 Å molecular sieve prior use. Toluene was distilled from potassium and stored over 3 Å molecular sieve prior use.

In a 4 mL glass vial equipped with a perforated cap and a magnetic stirbar 60 μL pretreated diethyl-diallyl-malonate (0.25 mmol) and 0.19 mL toluene was filled together with 0.5 μmol A1 catalyst in (as 0.05 M stock solution in benzene-d6, 10 μL). After 4 h stirring at room temperature reaction mixture was taken out of the glovebox, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion/Yield^{b} | |
|---|---|---|
| mol % | % | TON^{c} |
| 0.1 | 99 | 494 |

^{a}moles of catalyst A1/initial moles of diethyl-diallyl-malonate × 50
^{b}GC-FID area of 1,1-diethyl cyclopent-3-ene-1,1-dicarboxylate/(GC-FID area of diethyl-diallyl-malonate + GC-FID area of 1,1-diethyl cyclopent-3-ene-1,1-dicarboxylate)
^{c}Conversion/Cat. loading/2

Use of Catalyst A2 in Self-Metathesis of Methyl-Dec-9-Enoate

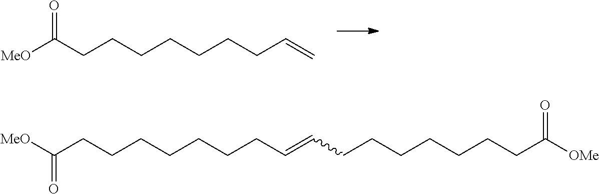 [SMILES: C=CCCCCCCCC(=O)OC ; COC(=O)CCCCCCCC=CCCCCCCCC(=O)OC]

Reaction Type:

Methyl dec-9-enoate self-metathesis

Catalyst: A2

Experimental:

Pretreatment: Methyl-dec-9-enoate was taken into glovebox, percolated on an alumina pad of 20% alumina and stored over 3 Å molecular sieve prior use.

In a 4 mL glass vial equipped with a perforated cap and a magnetic stirbar 165 μL (0.79 mmol) pretreated methyl-9-decenote was filled together with 0.5 μmol catalyst A2 (as 0.025 M stock solution in benzene-d6, 20 μL). After 4 h stirring at room temperature ca. 100 μL sample was taken out of the glovebox, measured by weight, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion^{b} | Yield^{c} | | |
|---|---|---|---|---|
| mol % | % | % | TON^{d} | Z/E^{e} |
| 0.06 | 56 | 56 | 441 | 33/67 |

^{a}moles of catalyst A2/moles of methyl-dec-9-enoate × 100
^{b}100 × (1 − moles of methyl-dec-9-enoate in product/initial moles of methyl-dec-9-enoate)
^{c}moles of dimethyl-octadec-9-enedioate/initial moles of methyl-dec-9-enoate × 50
^{d}moles of dimethyl-octadec-9-enedioate/moles of catalyst A2 × 2
^{e}ratio of (Z)- and (E)-dimethyl-octadec-9-enedioate

Use of Catalyst A2 in Self-Metathesis of Allylbenzene

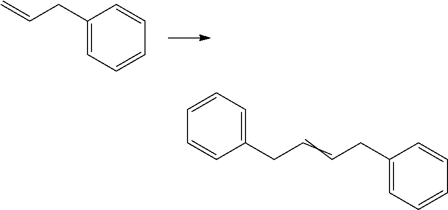

Reaction Type:

Allylbenzene self-metathesis

Catalyst: A2

Experimental:

Pretreatment: Allylbenzene was taken into glovebox, percolated on an alumina pad of 20% alumina and stored over 3 Å molecular sieve prior use.

In a 4 mL glass vial equipped with a perforated cap and a magnetic stirbar 132 μL (1 mmol) pretreated allylbenzene was filled together with 1 μmol A2 catalyst in (as 0.025 M stock solution in benzene-d6, 40 μL). After 4 h stirring at room temperature ca. 100 μL sample was taken out of the glovebox, measured by weight, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion | Yield^{c} | | |
|---|---|---|---|---|
| mol % | % | % | TON^{d} | E/Z^{e} |
| 0.1 | 23 | 23 | 117 | 59/41 |

^{a}moles of catalyst A2/initial moles of allylbenzene × 100
^{b}100 × (1 − moles of allylbenzene in product/initial moles of allylbenzene)
^{c}moles of 1,4-diphenyl-but-2-ene/initial moles of allylbenzene × 50
^{d}moles of 1,4-diphenyl-but-2-ene/moles of catalyst A2 × 2
^{e}ratio of (Z)- and (E)- 1,4-diphenyl-but-2-ene

Use of Catalyst A2 in RCM of Diethyl-Diallyl-Malonate

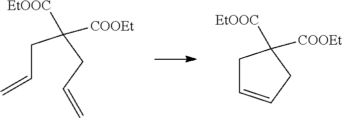 [SMILES: C=CCC(CC=C)(C(=O)OCC)C(=O)OCC ; CCOC(=O)C1(C(=O)OCC)CC=CC1]

Reaction Type:

Diethyl-diallyl-malonate ring closing metathesis

Catalyst: A2

Experimental:

Pretreatment: Diethyl-diallyl-malonate was taken into glovebox, percolated on an alumina pad of 20% alumina and stored over 3 Å molecular sieve prior use. Toluene was distilled from potassium and stored over 3 Å molecular sieve prior use.

In a 4 mL glass vial equipped with a perforated cap and a magnetic stirbar 60 μL pretreated diethyl-diallyl-malonate (0.25 mmol) and 0.19 mL toluene was filled together with 0.5 μmol A2 catalyst in (as 0.025 M stock solution in benzene-d6, 20 μL). After 4 h stirring at room temperature reaction mixture was taken out of the glovebox, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion/Yield^{b} | |
|---|---|---|
| mol % | % | TON^{e} |
| 0.1 | 31 | 157 |

^{a}moles of catalyst A2/initial moles of diethyl-diallyl-malonate × 50
^{b}GC-FID area of 1,1-diethyl cyclopent-3-ene-1,1-dicarboxylate/(GC-FID area of diethyl-diallyl-malonate + GC-FID area of 1,1-diethyl cyclopent-3-ene-1,1-dicarboxylate)
^{c}Conversion/Cat. loading/2

Use of Catalyst A5 in Self-Metathesis of Methyl-Dec-9-Enoate

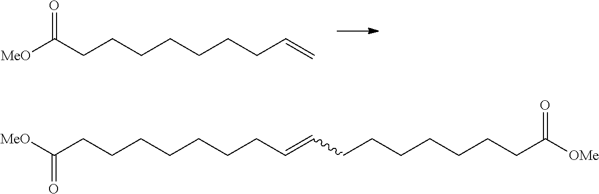 [SMILES: C=CCCCCCCCC(=O)OC ; COC(=O)CCCCCCCC=CCCCCCCCC(=O)OC]

Reaction Type:

Methyl dec-9-enoate self-metathesis

Catalyst: A5

Experimental:

Pretreatment: Methyl-dec-9-enoate was taken into glovebox, percolated on an alumina pad of 20% alumina and stored over 3 Å molecular sieve prior use.

In a 4 mL glass vial equipped with a perforated cap and a magnetic stirbar 165 μL (0.79 mmol) pretreated methyl-9-decenote was filled together with 0.5 μmol catalyst A5 (as 0.1 M stock solution in benzene-d6, 5 μL). After 4 h stirring at room temperature ca. 100 μL sample was taken out of the glovebox, measured by weight, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion^{b} | Yield^{c} | | |
|---|---|---|---|---|
| mol % | % | % | TON^{d} | Z/E^{e} |
| 0.06 | 92 | 92 | 727 | 21/79 |

^{a}moles of catalyst A5/moles of methyl-dec-9-enoate × 100
^{b}100 × (1 − moles of methyl-dec-9-enoate in product/initial moles of methyl-dec-9-enoate)
^{c}moles of dimethyl-octadec-9-enedioate/initial moles of methyl-dec-9-enoate × 50
^{d}moles of dimethyl-octadec-9-enedioate/moles of catalyst A5 × 2
^{e}ratio of (Z)- and (E)-dimethyl-octadec-9-enedioate

Use of Catalyst A5 in Self-Metathesis of Allylbenzene

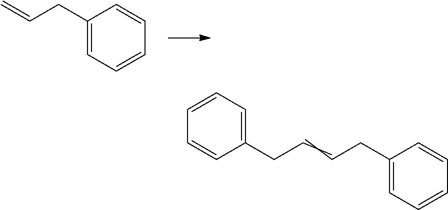

Reaction Type:

Allylbenzene self-metathesis

Catalyst: A5

Experimental:

Pretreatment: Allylbenzene was taken into glovebox, percolated on an alumina pad of 20% alumina and stored over 3 Å molecular sieve prior use.

In a 4 mL glass vial equipped with a perforated cap and a magnetic stirbar 132 μL (1 mmol) pretreated allylbenzene was filled together with 1 μmol A5 catalyst in (as 0.1 M stock solution in benzene-d6, 10 μL). After 4 h stirring at room temperature ca. 100 μL sample was taken out of the glovebox, measured by weight, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion | Yield^{c} | | |
|---|---|---|---|---|
| mol % | % | % | TON^{d} | E/Z^{e} |
| 0.1 | 74 | 74 | 367 | 52/48 |

^{a}moles of catalyst A5/initial moles of allylbenzene × 100
^{b}100 × (1 − moles of allylbenzene in product/initial moles of allylbenzene)
^{c}moles of 1,4-diphenyl-but-2-ene/initial moles of allylbenzene × 50
^{d}moles of 1,4-diphenyl-but-2-ene/moles of catalyst A5 × 2
^{e}ratio of(Z)- and (E)- 1,4-diphenyl-but-2-ene

Use of Catalyst A5 in RCM of Diethyl-Diallyl-Malonate

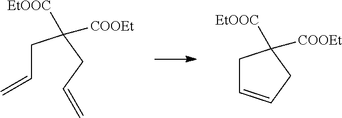 [SMILES: C=CCC(CC=C)(C(=O)OCC)C(=O)OCC ; CCOC(=O)C1(C(=O)OCC)CC=CC1]

Reaction Type:

Diethyl-diallyl-malonate ring closing metathesis

Catalyst: A5

Experimental:

Pretreatment: Diethyl-diallyl-malonate was taken into glovebox, percolated on an alumina pad of 20% alumina and stored over 3 Å molecular sieve prior use. Toluene was distilled from potassium and stored over 3 Å molecular sieve prior use.

In a 4 mL glass vial equipped with a perforated cap and a magnetic stirbar 60 μL pretreated diethyl-diallyl-malonate (0.25 mmol) and 0.19 mL toluene was filled together with 0.5 μmol A5 catalyst in (as 0.1 M stock solution in benzene-d6, 5 μL). After 4 h stirring at room temperature reaction mixture was taken out of the glovebox, and following GC standards were added: 1 mL mesitylene in EtOAc solution and 1 mL pentadecane in EtOAc solution, both of 60.0 mg/mL. This solution was poured onto the top of a silica pad (1 mL silica), eluted with further 8 mL EtOAc, and from the collected elute 100 μL was analyzed by GC-FID and GC-MS.

Results:

| Cat. loading^{a} | Conversion/Yield^{b} | |
|---|---|---|
| mol % | % | TON^{e} |
| 0.1 | 99 | 497 |

^{a}moles of catalyst A5/initial moles of diethyl-diallyl-malonate × 50
^{b}GC-FID area of 1,1-diethyl cyclopent-3-ene-1,1-dicarboxylate/(GC-FID area of diethyl-diallyl-malonate + GC-FID area of 1,1-diethyl cyclopent-3-ene-1,1-dicarboxylate)
^{c}Conversion/Cat. loading/2

### Preparation of M(NR¹)[═C(R²)(R³)](OR′)[OC(O)R′] Compounds

The reaction between bisalkoxide Mo(NAr)(CHCMe₂Ph)(OR_{F6})₂ and one equivalent of 2,6-bis(4′-methylphenyl)benzoic acid (Ter_{Me}CO₂H) yielded Mo(NAr)(CHCMe₂Ph)(OR_{F6})(O₂CTer_{Me}), B1, in moderate yield as orange crystals:

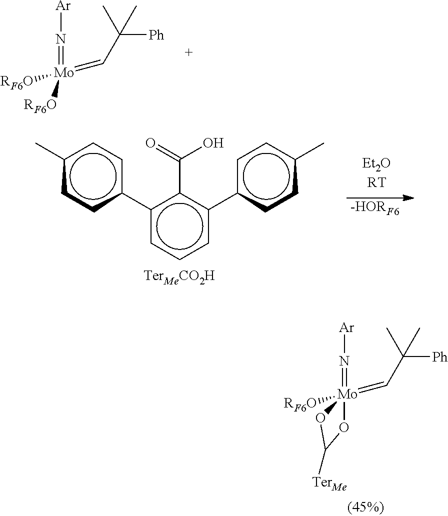

It is noted that to obtain the product, reaction concentration needs be carefully controlled. If the reaction is too concentrated or if the solution containing the carboxylic acid is added too quickly, the yield can be low, and, in the most extreme case, insufficient to isolate.

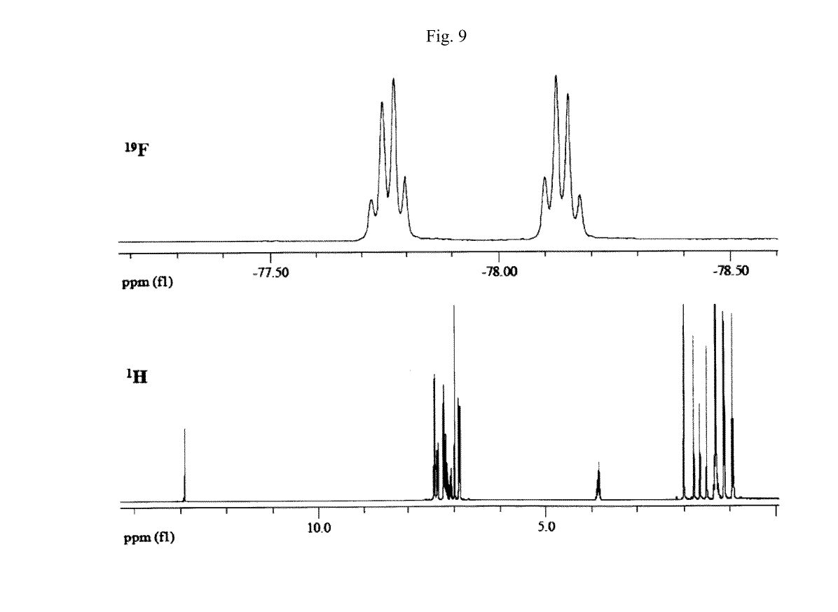

The proton NMR spectrum of B1 shows one alkylidene peak at 13.07 ppm and a set of quartets in its ¹⁹F NMR spectrum, which is consistent with C₁ symmetry at the metal center. The ¹⁹F NMR feature is very characteristic of species that contain only one OR_{F6} ligand and serves as the indicative spectroscopic signature that such species have been made (FIG. 9).

Reactivity Data

In this section the reactivity of species 1-8 were compared to the analgous SAM species (Mo(NAr)(CHCMe₂Ph)(Me₂Pyr)(O₂CTer_{Me}), C1a, Me₂Pyr=2,5-Me₂C₄H₂N)) and (Mo(NR)(CHCMe₂Ph)(Pyr)(OHMT) (R=Ar, 2a; R=Ar′, 3a; R=Ar^{iPr}, 4a; R=Ad, 5a; Pyr=pyrrolide)) complexes. The reactions screened are the ring closing metathesis (RCM) of diallyl ether, the homocouplings of 1-hexene and 1-octene, and the ring opening metathesis polymerization (ROMP) of 2,3-dicarbomethoxynorbonadiene (DCMNBD).

### 1: Mo(NAr)(CHCMe₂Ph)(OR_{F6})(O₂CTer_{Me});

### 2: Mo(NAr)(CHCMe₂Ph)(OR_{F6})(OHMT);

### 3: Mo(NAr′)(CHCMe₂Ph)(OR_{F6})(OHMT);

### 4: Mo(NAr^{iPr})(CHCMe₂Ph)(OR_{F6})(OHMT);

### 5: Mo(NAd)(CHCMe₂Ph)(OR_{F6})(OHMT);

### 6: Mo(NAr′)(CHCMe₂Ph)(OR_{F6})[N(H)HMT];

### 7: Mo(NAr^{iPr})(CHCMe₂Ph)(OR_{F6})[N(H)HMT];

### 8: Mo(NAd)(CHCMe₂Ph)(OR_{F6})(HMT)

RCM of Diallyl Ether

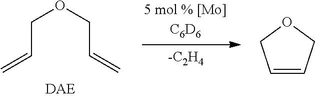

Compounds 1-8, 1a, and 2a-5a were evaluated by reacting 11 μmoles of each catalyst with 20 equivalents of DAE in 0.7 mL of C₆D₆ within a Teflon-sealed J-Young tube. The reactions were monitored during a period of 20-60 min and 15 h after mixing and the results are shown in Table 11.

TABLE 11
RCM Reaction Data

| | | | | | Eq. | Conversion, |
|---|---|---|---|---|---|---|
| Catalyst | R | X | Y | [Mo], mM | Subs. | Time |
| 1 | Ar | OR_{F6} | O₂CTer_{Me} | 15 | 20 | 91%, 20 m |
| | | | | | | 98%, 2 h |
| | | | | | | 98%, 15 h |
| 1a | Ar | Me₂Pyr | O₂CTer_{Me} | 17 | 20 | 33%, 30 m |
| | | | | | | 94%, 2 h |
| | | | | | | 96%, 22 h |
| 2 | Ar | OR_{F6} | OHMT | 15 | 20 | 91%, 40 m |
| | | | | | | 91%, 2.5 h |
| | | | | | | 91%, 15 h |
| 2a | Ar | Pyr | OHMT | 17 | 20 | 97%, 30 m |
| | | | | | | 97%, 2.3 h |
| | | | | | | 97%, 15 h |
| 3 | Ar′ | OR_{F6} | OHMT | 15 | 20 | 95%, 40 m |
| | | | | | | 95%, 2.5 h |
| | | | | | | 95%, 15 h |
| 3a | Ar′ | Pyr | OHMT | 18 | 20 | 95%, 40 m |
| | | | | | | 95%, 2.3 h |
| | | | | | | 96%, 15 h |
| 4 | Ar^{iPr} | OR_{F6} | OHMT | 15 | 20 | 96%, 40 m |
| | | | | | | 96%, 2.5 h |
| | | | | | | 96%, 15 h |
| 4a | Ar^{iPr} | Pyr | OHMT | 18 | 20 | 93%, 40 m |
| | | | | | | 93%, 2.3 h |
| | | | | | | 93%, 15 h |
| 5 | Ad | OR_{F6} | OHMT | 15 | 20 | 2%, 50 m |
| | | | | | | 4%, 2.6 h |
| | | | | | | 12%, 15 h |
| 5a | Ad | Pyr | OHMT | 17 | 20 | 94%, 1 h |
| | | | | | | 94%, 2.5 h |
| | | | | | | 94%, 15 h |
| 6 | Ar′ | OR_{F6} | N(H)HMT | 16 | 20 | 35%, 30 m |
| | | | | | | 50%, 2.5 h |
| | | | | | | 69%, 12 h |
| 7 | Ar^{iPr} | OR_{F6} | N(H)HMT | 15 | 20 | 57%, 30 m |
| | | | | | | 64%, 2.5 h |
| | | | | | | 65%, 12 h |
| 8 | Ad | OR_{F6} | HMT | 15 | 20 | 26%, 50 m |
| | | | | | | 87%, 15 h |
| | | | | | | 93%, 22 h |

Homocoupling of 1-Hexene

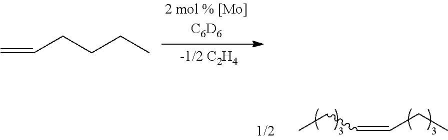

Activity were tested by reacting 11 μmoles of each catalyst with 50 equivalents of 1-hexene in 1.5 mL of C₆D₆ in an open vial under inert-gas atmosphere. The vial was kept open to allow removal of ethylene gas (a byproduct of the reaction) and to reach complete conversion. The reactions were stirred at RT for 1 h, with the exception of 2 cases. The reaction was evaluated by ¹H and ¹³C NMR and the results are shown in Table 12.

TABLE 12
Homocoupling of 1-hexene.

| | | | | [Mo], | Eq. | |
|---|---|---|---|---|---|---|
| Catalyst | R | X | Y | mM | Subs. | Conversion, Time |
| 1 | Ar | OR_{F6} | O₂CTer_{Me} | 8 | 50 | 98%, 84% trans, 1 h |
| 1a | Ar | Me₂Pyr | O₂CTer_{Me} | 9 | 50 | 82%, 53% trans, 1 h |
| 2 | Ar | OR_{F6} | OHMT | 8 | 50 | 99%, 84% trans, 1 h |
| 2a | Ar | Pyr | OHMT | 9 | 50 | 99%, 82% trans, 1 h |
| 3 | Ar′ | OR_{F6} | OHMT | 8 | 50 | 99%, 86% trans, 1 h |
| 3a | Ar′ | Pyr | OHMT | 9 | 50 | 99%, 86% trans, 1 h |
| 4 | Ar^{iPr} | OR_{F6} | OHMT | 8 | 50 | 99%, 87% trans, 1 h |
| 4a | Ar^{iPr} | Pyr | OHMT | 9 | 50 | 99%, 81% trans, 1 h |
| 5 | Ad | OR_{F6} | OHMT | 8 | 50 | 71%, 3 h |
| | | | | | | 74%, 54% trans, 5 h |
| | | | | | | — |
| 5a | Ad | Pyr | OHMT | 9 | 50 | 92%, 89% trans, 1 h |
| 6 | Ar′ | OR_{F6} | N(H)HMT | 8 | 50 | 96%, 83% trans, 1 h |
| 7 | Ar^{iPr} | OR_{F6} | N(H)HMT | 7 | 50 | 94%, 85% trans, 1 h |
| 8 | Ad | OR_{F6} | HMT | 8 | 50 | 10%, 5 h |

Homocoupling of 1-Octene

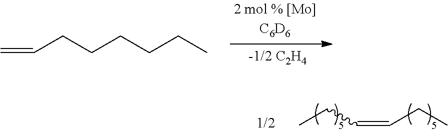

The reactions were carried out with 6 μmoles of each catalyst and 50 equivalents of 1-octene in 0.75 mL of C₆D₆ in an open vial under inert-gas atmosphere and the reaction was evaluated by ¹H NMR in CDCl₃. The results are shown in Table 13.

TABLE 13
Homocoupling of 1-octene

| Catalyst | R | X | Y | [Mo], mM | Eq. Subs. | Conversion, Time |
|---|---|---|---|---|---|---|
| 1 | Ar | OR_{F6} | O₂CTer_{Me} | 7.5 | 50 | 99% conv, 83% trans, 1 h |
| 1a | Ar | Me₂Pyr | O₂CTer_{Me} | 8.3 | 50 | 16% conv, 57% trans, 4 h |
| 2 | Ar | OR_{F6} | OHMT | 7.4 | 50 | 99% conv, 81% trans, 1 h |
| 2a | Ar | Pyr | OHMT | 8.3 | 50 | 99% conv, 82% trans, 1 h |
| 3 | Ar′ | OR_{F6} | OHMT | 7.8 | 50 | 99% conv, 80% trans, 1 h |
| 3a | Ar′ | Pyr | OHMT | 9.0 | 50 | 99% conv, 85% trans, 1 h |
| 4 | Ar^{iPr} | OR_{F6} | OHMT | 7.7 | 50 | 99% conv, 81% trans, 1 h |
| 4a | Ar^{iPr} | Pyr | OHMT | 8.6 | 50 | 99% conv, 73% trans, 1 h |
| 5 | Ad | OR_{F6} | OHMT | 7.5 | 50 | 26% conv, 62% cis, 1 h |
| | | | | | | 59% conv, 55% cis, 4 h |
| | | | | | | 75% conv, 52% cis, 5 h |
| 5a | Ad | Pyr | OHMT | 13.8 | 50 | 99% conv, 82% trans, 1 h |
| 6 | Ar′ | OR_{F6} | N(H)HMT | 7.8 | 50 | 99% conv, 83% trans, 1 h |
| 7 | Ar^{iPr} | OR_{F6} | N(H)HMT | 7.7 | 50 | 99% conv, 82% trans, 1 h |
| 8 | Ad | OR_{F6} | HMT | 7.7 | 50 | Slow |

ROMP of DCMNBD

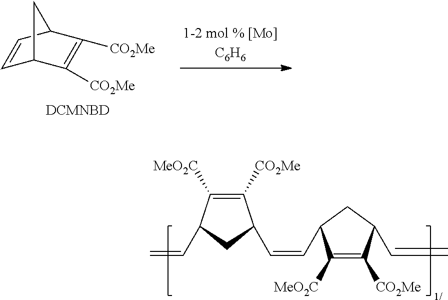

The reactions were carried out using 6 μmoles of catalyst and 50 equivalents of DCMNBD in 1.2 mL of toluene at RT. The resulting polymer was analyzed by ¹H and ¹³C NMR methods and the results are shown in Table 14.

TABLE 14
ROMP of DCMNBD

| Catalyst | R | X | Y | [Mo], mM | Eq. Subs. | Polymer Structure |
|---|---|---|---|---|---|---|
| 1 | Ar | OR_{F6} | O₂CTer_{Me} | 4.7 | 50 | Slow |
| 1a | Ar | Me₂Pyr | O₂CTer_{Me} | 5.2 | 50 | Slow |
| 2 | Ar | OR_{F6} | OHMT | 4.6 | 50 | >98% cis; 78% isotactic |
| 2a | Ar | Pyr | OHMT | 5.2 | 50 | >98% cis, syndiotactic |
| 3 | Ar′ | OR_{F6} | OHMT | 4.9 | 50 | 95% cis, 73% syndiotactic |
| 3a | Ar′ | Pyr | OHMT | 5.6 | 50 | >98% cis, syndiotactic |
| 4 | Ar^{iPr} | OR_{F6} | OHMT | 4.8 | 50 | 98% cis, 95% syndiotactic |
| 4a | Ar^{iPr} | Pyr | OHMT | 5.4 | 50 | >98% cis, syndiotactic |
| 5 | Ad | OR_{F6} | OHMT | 4.7 | 50 | 90% cis, 76% syndiotactic |
| 5a | Ad | Pyr | OHMT | 8.6 | 100 | >98% cis, syndiotactic |
| 6 | Ar′ | OR_{F6} | N(H)HMT | 4.9 | 50 | 95% cis, 71% isotactic |
| 7 | Ar^{iPr} | OR_{F6} | N(H)HMT | 4.8 | 50 | 90% cis, 54% isotactic |
| 8 | Ad | OR_{F6} | HMT | 4.8 | 100 | 83% cis, 91% syndiotactic |
| | | | | | | (5 d) |

Experimental:

All reactions and manipulations of air and moisture sensitive compounds were handled in oven-dried glassware (150° C., 2 h) under a N₂ atmosphere either in a dual Schlenk line or in vacuum atmosphere glove box. HPLC grade solvents (benzene, toluene, diethyl ether, tetrahydrofuran, pentane, and methylene chloride), were purge with N₂ and passed through activated alumina and stored over molecular sieves 12 hours prior to use. 1,2-dimethoxyethane was dried in an oven-dried Schlenk flask with sodium and benzophenone ketal, vacuumed transferred into another oven-dried Schlenk flask and stored over molecular sieves 12 hours prior to use. n-BuLi, F₉OH were bought from VWR and used as received. Diallyl ether, 1-hexene, and 1-octene were bought from Sigma-Aldrich and dried over activated 3 Å molecular sieves overnight before use. DCMNBD (Tabor, D. C.; White, F. H.; Collier, L. W.; Evans, S. A. J. Org. Chem. 1983, 48, 1638), Mo(NAd)(CHCMe₂Ph)(OTf)₂(DME) and Mo(NR)(CHCMe₂Ph)(OR_{F6})₂ complexes (Oskam, J. H.; Fox, H. H.; Yap, K. B.; McConville, D. H.; O'Dell, R.; Lichtenstein, B. J.; Schrock, R. R. Jour. Organomet. Chem. 1993, 459, 185), LiOHMT, LiN(H)HMT, LiHMT, LiTIPT and LiHIPT (Schiemenz, B.; Power, P. P. Organometallics 1996, 15, 958) were prepared according to literature procedures. Benzene-d₆ was stored over molecular sieves 12 hours prior to use. All NMR spectra were recorded with a Bruker 400 MHz spectrometer. Elemental analyses were performed by Midwest Microlab, LLC.

Mo(NAr)(CHCMe₂Ph)(OR_{F6})(O₂CTer_{Me}) (1).

Mo(NAr)(CHCMe₂Ph)(OR_{F6})₂ (0.207 g, 0.270 mmol) was dissolved in Et₂O (5.00 mL) and cooled down to −35° C. for 1 h. In a separate vial, Ter_{Me}CO₂H (0.082 g, 0.270 mmol) was dissolved in Et₂O (5.00 mL) and also cooled down to −35° C. for 1 h. Then, the carboxylic acid solution was added dropwise to the bisalkoxide solution and left stirring at RT for 1 h. The volatiles were removed under reduced pressure and the crude was dissolved in a minimal amount of pentane and place at −35° C. for a few days to generate orange crystals of pure 1 (0.109 g, 45%): ¹H NMR (400 MHz, C₆D₆) δ 13.07 (s, 1H, Mo=CH, J_{CH}=125.8 Hz), 7.59 (d, 4H, p-tolyl), 7.52 (dd, 2H, aromatic), 7.42-7.28 (overlapping peaks, 7H, aromatic), 7.23 (td, 1H, aromatic), 7.17-7.14 (t, 1H, aromatic), 4.02 (sept, 2H, CHMe₂), 2.17 (s, 6H, p-MeC₆H₄), 1.95 (s, 3H, CH₃), 1.82 (s, 3H, CH₃), 1.67 (s, 3H, CH₃), 1.48 (d, 6H, CHMe₂), 1.29 (d, 6H, CHMe₂); ¹³C NMR (100 MHz, C₆D₆) δ 289.3 (Mo=CHCMe₂Ph), 189.3 (Ter_{Me}CO₂Mo), 152.4, 149.2, 149.2, 140.9, 138.0, 137.6, 134.0, 130.2, 129.5, 128.8, 128.7, 128.6, 128.6, 126.6, 126.3, 123.2, 55.3, 32.4, 29.3, 28.5, 24.4, 23.5, 20.9, 18.3; ¹⁹F NMR (376 MHz, C₆D₆) δ −77.7 (q, 3F, CF₃), −78.1 (q, 3F, CF₃). Anal. Calcd for C₄₇H₄₉F₆MoNO₃: C, 63.72; H, 5.58, N, 1.58. Found: C, 63.54; H, 5.31; N, 1.47.

RCM of DAE

Complexes 1-8 and 1a-5a (0.010 g, 0.011 mmol) were dissolved in 0.70 mL of C₆D₆ and place inside a J-Young tube. Then, DAE (0.022 g, 0.230 mmol) was added to it and the NMR tube was sealed and monitored over the course of 1 day.

Homocoupling of 1-Hexene

Complexes 1-8 and 1a-5a (0.010 g, 0.011 mmol) were dissolved in 1.50 mL of C₆D₆ in a vial. Then, 1-hexene (0.075-0.080 mL, 0.570 mmol) was added to it and the solution was stirred in an open vial, under a nitrogen-filled atmosphere, for the indicated time. The solutions were placed in a J-Young tube to monitor its progress by ¹H and ¹³C NMR, based on the spectra of pure cis- and trans-5-decene obtained from Sigma-Aldrich.

Homocoupling of 1-Octene

Complexes 1-8 and 1a-5a (0.005 g, 0.006 mmol) were dissolved in 0.75 mL of C₆D₆ in a vial. Then, 1-octene (0.043-0.050 mL, 0.300 mmol) was added to it and the solution was stirred in an open vial, under a nitrogen-filled atmosphere, for the indicated time. An aliquot was then dissolved in CDCl₃ to monitor its progress by ¹H NMR.

ROMP of DCMNBD

Complexes 1-8 and 1a (0.005 g, 0.006 mmol) were dissolved in 0.50 ml of toluene and added to a stirring toluene solution of DCMNBD (0.70 mL, 50 eq, 0.300 mmol). The solution was left stirring for 2 h until it formed a gel. Then, the gel was redissolved in CH₂Cl₂ (7.00 mL) and quenched with benzaldehyde (0.100 mL) before precipitation from methanol (60.0 mL) and isolation by vacuum filtration. The dry polymer was examined by ¹H and ¹³C NMR to determine the tacticity and cis content according to known literature reports.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A compound of formula I: 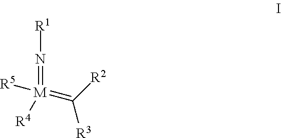
wherein:
M is molybdenum or tungsten;
R¹ is an optionally substituted group selected from C₁₋₂₀ aliphatic, C₁₋₂₀ heteroaliphatic having 1-3 heteroatoms independently selected from nitrogen, oxygen, or sulfur, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
each of R² and R³ is independently R, —OR, —SR, —N(R)₂, —OC(O)R, —SOR, —SO₂R, —SO₂N(R)₂, —C(O)N(R)₂, —NRC(O)R, or —NRSO₂R;
R⁴ is —OR^{s};
R^{s} is —C(R^{t})₂—R′, —Ar^{a}, or an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
each R^{t} is independently halogen or R;
R⁵ is different from R⁴, and is —OR′, —OC(O)R′, —N(R′)₂, or R″;
R′ is hydrogen, —Ar^{a}, or an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R″ is —Ar^{a}, or an optionally substituted group selected from phenyl, an 8-10 membered bicyclic aryl ring, a 5-6 membered monocyclic heteroaryl ring having 1-4 heteroatoms independently selected from nitrogen, oxygen, or sulfur, or an 8-10 membered bicyclic heteroaryl ring having 1-5 heteroatoms independently selected from nitrogen, oxygen, or sulfur;
Ar^{a} is of the following formula: 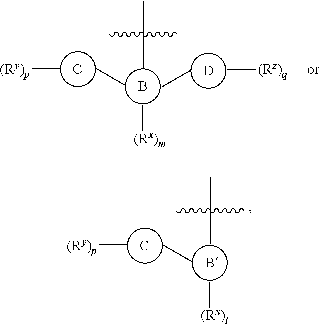
wherein:
m is 0-3;
Ring B is an optionally substituted group selected from phenyl or a 5-6 membered monocyclic heteroaryl ring having 1-4 heteroatoms independently selected from nitrogen, oxygen, or sulfur;
each of p and q is independently 0-5;
t is 0-4;
each of Ring B′, Ring C and Ring D is independently an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-14 membered bicyclic or tricyclic heteroaryl ring having 1-5 heteroatoms independently selected from nitrogen, oxygen, or sulfur;
each of R^{x}, R^{y}, and R^{z} is independently halogen, R, —OR, —SR, —S(O)R, —S(O)₂R, —OSi(R)₃, —N(R)₂, —NRC(O)R, —NRC(O)OR, —NRC(O)N(R)₂, —NRSO₂R, —NRSO₂N(R)₂, or —NROR;
each R is independently hydrogen or an optionally substituted group selected from C₁₋₂₀ aliphatic, C₁₋₂₀ heteroaliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 3-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur; or:
two R groups on the same atom are optionally taken together with the atom to which they are attached to form an optionally substituted 3-10 membered, monocyclic or bicyclic, saturated, partially unsaturated, or aryl ring having, in addition to the atom to which they are attached, 0-4 heteroatoms independently selected from nitrogen, oxygen, or sulfur.

2. The compound of claim 1, wherein R¹ is adamantyl.

3. The compound of claim 1, wherein R¹ is optionally substituted phenyl.

4-6. (canceled)

7. The compound of claim 1, wherein one of R² and R³ is hydrogen and the other is optionally substituted C₁₋₂₀ aliphatic.

8. (canceled)

9. The compound of claim 1, wherein R⁴ is —O—C(R^{t})₂—R′.

10. The compound of claim 1, wherein R⁴ is —OAr^{a}.

11-13. (canceled)

14. The compound of claim 1, wherein R⁵ is —OR′, and R′ is not hydrogen.

15. The compound of claim 14, wherein R′ is R″.

16. The compound of claim 15, wherein R″ is optionally substituted phenyl.

17. The compound of claim 15, wherein R″ is —Ar^{a}.

18. The compound of claim 1, wherein R⁵ is —OC(O)R′.

19-20. (canceled)

21. The compound of claim 1, wherein R⁵ is —N(R′)₂.

22-24. (canceled)

25. The compound of claim 1, wherein R⁵ is R″, and R⁵ is bonded to M through an aromatic carbon atom.

26. The compound of claim 25, wherein R⁵ is Ar^{a}.

27. The compound of claim 1, wherein the compound is selected from:
Mo(NAd)(CHCMe₂Ph)(OHIPT)(OCMe₃), Mo(NAr)(CHCMe₂Ph)(OR_{F6})(OHMT), Mo(NAr′)(CHCMe₂Ph)(OR_{F6})(OHMT), Mo(NAr^{iPr})(CHCMe₂Ph)(OR_{F6})(OHMT), Mo(NAd)(CHCMe₂Ph)(OR_{F6})(OHMT), Mo(NAd)(CHCMe₂Ph)(OR_{F6})[N(H)HMT)], Mo(NAr′) (CHCMe₂Ph)(OR_{F6})[N(H)HMT)], Mo(NAr^{iPr})(CHCMe₂Ph)(OR_{F6})[N(H)HMT)], Mo(NAr)(CHCMe₂Ph)(OR_{F6})[N(H)HMT)], Mo(NAr)(CHCMe₂Ph)(OR_{F6})(O₂CTer_{Me}), 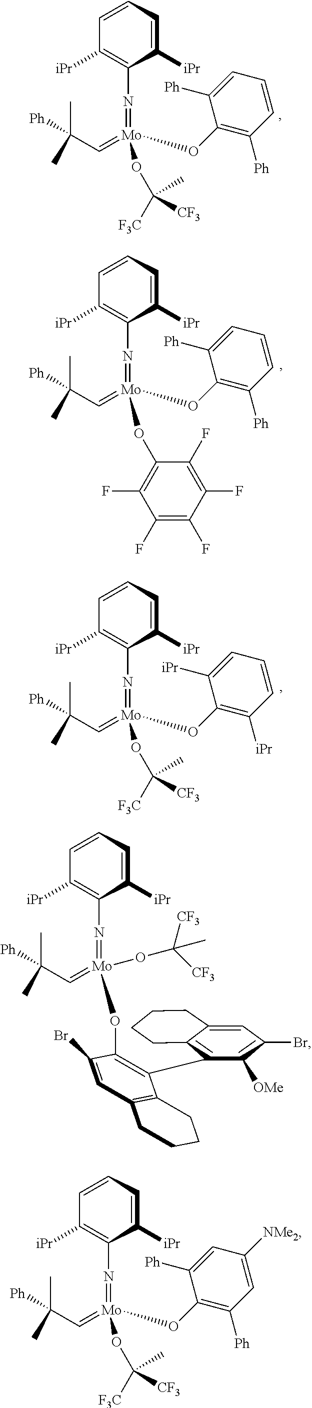 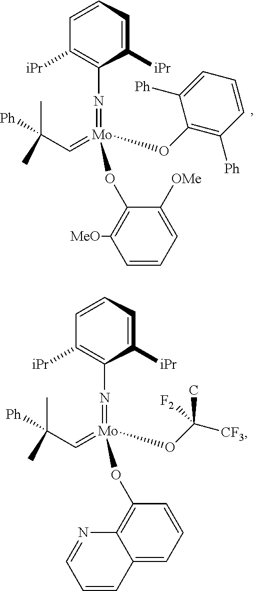
Mo(NAd)(CHCMe₂Ph)(OR_{F6})(HMT), Mo(NAr^{m})(CHCMe₂Ph)(OR_{F6})(HMT), Mo(NAr′)(CHCMe₂Ph)(OR_{F6})(HMT), Mo(NAr)(CHCMe₂Ph)(OR_{F6})(HMT), Mo(NAd)(CHCMe₂Ph)(OR_{F6})(TIPT), Mo(NAr′)(CHCMe₂Ph)(OR_{F6})(TIPT), Mo(NAr)(CHCMe₂Ph)(OR_{F6})(TIPT), and Mo(NAd)(CHCMe₂Ph)(OR_{F9})(HMT), wherein Ad is 1-adamantyl, OHIPT is 2,6-bis(2′ 4′,6′-triisopropylphenyl)phen-2-oxide, Ar is 2,6-diisopropylphenyl, OR_{F6} is OCMe(CF₃)₂, OHMT is 2,6-bis(2′,4′,6′-trimethylphenyl)phenoxide, Ar′ is 2,6-dimethylphenyl, Ar^{iPr} is 2-isopropylphenyl, HMT is 2,6-bis(2′,4′,6′-trimethylphenyl)phenyl, Ter_{Me} is 2,6-bis(4′-methylphenyl)phenyl, Ar^{m} is 3,5-dimethylphenyl, and TIPT is 2,2′,6,6′-tetraisopropylterphenyl.

28. A method for preparing a compound of claim 1, comprising:
a) providing a compound of formula II: 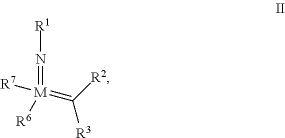
wherein each of R⁶ and R⁷ is independently optionally substituted pyrrolide;
b) reacting the compound of formula II with a compound having the structure of R⁴H, or a salt thereof, to provide a compound of formula III: 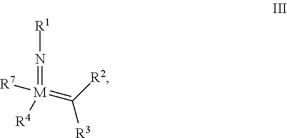
c) reacting the compound of formula III with a compound having the structure of R⁵H, or a salt thereof, to provide a compound of formula I.

29. A method for preparing a compound of claim 1, comprising:
a) providing a compound of formula II: 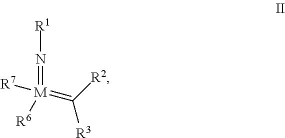
b) reacting the compound of formula II with a compound having the structure of R⁵H, or a salt thereof, to provide a compound of formula III′: 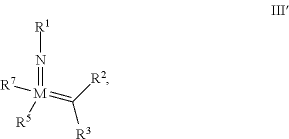
c) reacting the compound of formula III′ with a compound having the structure of R⁴H, or a salt thereof, to provide a compound of formula I.

30. A method for preparing a compound of claim 1, comprising:
a) providing a compound of formula II′: 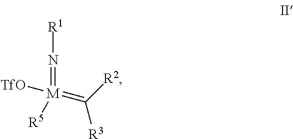
b) reacting the compound of formula II′ with a compound having the structure of R⁴H, or a salt thereof, to provide a compound of formula I.

31. (canceled)

32. A method for preparing a compound of claim 1, comprising:
a) providing a compound of formula IV: 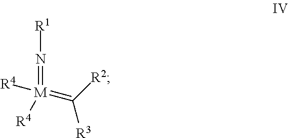
herein;
b) reacting the compound of formula IV with a compound having the structure of R⁵H, or a salt thereof, to provide a compound of formula I.

33-42. (canceled)

43. A method for performing a metathesis reaction, comprising providing a compound of claim 1.

44-47. (canceled)
